SEQUENCE LISTING (32 sequences)

SEQ ID NO: 1 - DNA, 18 bp, Artificial Sequence
  primer
tgactatgtcccgccact

SEQ ID NO: 2 - DNA, 22 bp, Artificial Sequence
  primer
tgatgcaaataaaaccggactg

SEQ ID NO: 3 - DNA, 21 bp, Artificial Sequence
  primer
accaagtttcttcagcttccg

SEQ ID NO: 4 - DNA, 21 bp, Artificial Sequence
  primer
ttgtccatcttgtcgtcttcg

SEQ ID NO: 5 - DNA, 22 bp, Artificial Sequence
  primer
gtcttgacctttaacaggaact

SEQ ID NO: 6 - DNA, 21 bp, Artificial Sequence
  primer
caaacaccttcctgacaccat

SEQ ID NO: 7 - DNA, 20 bp, Artificial Sequence
  primer
tctcccagctccagatacag

SEQ ID NO: 8 - DNA, 22 bp, Artificial Sequence
  primer
gctcttcgtcttcaatttcgtc

SEQ ID NO: 9 - DNA, 21 bp, Artificial Sequence
  primer
caacccagacatatccacctc

SEQ ID NO: 10 - DNA, 24 bp, Artificial Sequence
  primer
ctctgatcatctgaccaaaactca

SEQ ID NO: 11 - DNA, 21 bp, Artificial Sequence
  primer
gtttcttgctcgaagatgctc

SEQ ID NO: 12 - DNA, 22 bp, Artificial Sequence
  primer
caccaagtgcatcaaatacctg

SEQ ID NO: 13 - DNA, 21 bp, Artificial Sequence
  primer
ttcgggtaattctccatctgc

SEQ ID NO: 14 - DNA, 25 bp, Artificial Sequence
  primer
actattgtcttatgatcacgtttgc

SEQ ID NO: 15 - DNA, 22 bp, Artificial Sequence
  primer
aaagtcatctatgccatcacca

SEQ ID NO: 16 - DNA, 24 bp, Artificial Sequence
  primer
actgcacaagctatttttgatgac

SEQ ID NO: 17 - DNA, 22 bp, Artificial Sequence
  primer
atacctgcctcagaatttcctc

SEQ ID NO: 18 - DNA, 20 bp, Artificial Sequence
  primer
aatggaaggagagtgcttgg

SEQ ID NO: 19 - DNA, 22 bp, Artificial Sequence
  primer
gtcttgacctttaacaggaact

SEQ ID NO: 20 - DNA, 21 bp, Artificial Sequence
  primer
caaacaccttcctgacaccat

SEQ ID NO: 21 - DNA, 20 bp, Artificial Sequence
  primer
cttcctcttgtcacgctcag

SEQ ID NO: 22 - DNA, 24 bp, Artificial Sequence
  primer
ggcatccaagatacaaactcaaag

SEQ ID NO: 23 - DNA, 22 bp, Artificial Sequence
  primer
gcactggtacttcttgacatct

SEQ ID NO: 24 - DNA, 17 bp, Artificial Sequence
  primer
ggctgctacaaggccat

SEQ ID NO: 25 - DNA, 22 bp, Artificial Sequence
  primer
acattatccaggacatcactgc

SEQ ID NO: 26 - DNA, 21 bp, Artificial Sequence
  primer
gccatgtacattgacactgag

SEQ ID NO: 27 - DNA, 18 bp, Artificial Sequence
  primer
gcgttcgatgacagtggt

SEQ ID NO: 28 - DNA, 21 bp, Artificial Sequence
  primer
ctcccctcaacaacttctctg

SEQ ID NO: 29 - DNA, 23 bp, Artificial Sequence
  primer
agtctacttcctctgtgatgttg

SEQ ID NO: 30 - DNA, 23 bp, Artificial Sequence
  primer
gctataactggagagtgctgaag

SEQ ID NO: 31 - DNA, 22 bp, Artificial Sequence
  primer
aatactccacacgcaaatttcc

SEQ ID NO: 32 - DNA, 20 bp, Artificial Sequence
  primer
caagcagtcacagcacatga

(19) United States
(12) Patent Application Publication
(10) Pub. No.: US 2023/0416837 A1
(43) Pub. Date: Dec. 28, 2023

(54) COMPOSITIONS AND METHODS FOR IDENTIFICATION, ASSESSMENT AND TREATMENT OF CANCER PATIENT

(71) Applicant: AMBAY IMMUNE SENSORS AND CONTROLS, LLC/AMBAY, Hagerstown, MD (US)

(72) Inventors: Meena R. Chandok, Germantown, MD (US); Palanikumaran Sakthivel, Germantown, MD (US)

(21) Appl. No.: 18/252,399

(22) PCT Filed: Nov. 16, 2021
(86) PCT No.: PCT/US2021/059510

§ 371 Date: May 10, 2023

Related U.S. Application Data
(60) Provisional application No. 63/114,023, filed on Nov. 16, 2020.

(51) Int. Cl. C12Q 1/6886; C12Q 1/6851; G16B 25/10; G16B 40/20

(52) U.S. Cl. CPC C12Q 1/6886; C12Q 1/6851; G16B 25/10; G16B 40/20; C12Q 2600/158; C12Q 2600/106

(57) ABSTRACT

The present invention describes a non-invasive assay based on a key biomarkers that can be used to create a Radiation Response Index indicative of the tumor's response to radiation therapy prior to, during, and post-radiation therapy. The invention stratifies the subjects into clinically relevant categories, Radiation Resistant and Radiation Sensitive.

### STATEMENT REGARDING FEDERALLY SPONSORED RESEARCH & DEVELOPMENT

This invention was made with government support under NIH 75N91020000031 awarded by the National Institutes of Health. The government has certain rights in the invention.

### FIELD OF THE DISCLOSURE

The present disclosure is related to compositions and non-invasive/liquid biopsy methods for identifying a patient's response to cancer therapy, e.g. radiotherapy (RT), pre-treatment, during treatment, post-treatment, surveillance and modifying treatment accordingly.

This invention relates to the field of personalized radiotherapy (RT) and provides a clinically useful radiation response index (RRI) derived from a panel of biomarkers to (a) classify patients based on the radio-sensitivities of their tumor(s) to guide clinicians in designing treatment strategy in an individualized manner before the beginning of treatment; (b) monitor the efficacy of therapy during treatment to guide the adjustments in treatment regimens/adaptive RT; (c) monitor the outcome of therapy post-treatment to identify early relapse to guide timely alternative therapy options; (d) direct the use of other modalities (e.g., immunotherapy, molecular targeted therapy, hormone therapy) in combination with RT for patients who can't get the maximum benefit from RT alone. This invention also relates to the methods, systems and kits performing the testing for above mentioned scenarios (a-d).

### BACKGROUND

RT is a curative component of cancer treatment and being used to treat 30-70% of all cancer patients but resistance to RT is a major clinical challenge, which leads to tumor relapse. Not all patients that receive RT will obtain a therapeutic benefit; in the typical clinical treatment scenario, patients responding well to RT may demonstrate complete tumor regression at the irradiated site while the poor/weak responders to RT, develop locoregional recurrence shortly post-RT.

Treating cancer patients with a “one-size-fits-all” approach of a particular cancer therapy/RT and schedule has failed to deliver favorable results due to these deficiencies: (a) the inability to know in advance how the tumor will respond to therapy, (b) how to monitor if the therapy is working as expected and thereby implement a modification in the treatment in terms of dose adjustments/fractionation/duration to provide a patient optimal benefit, and (c) how to identify patients with a possibility of relapse post-treatment. Data indicates that up to 50% of prostate cancer patients undergoing radiation therapy experience recurrence of the disease within 5 years of treatment; hence the identification of early relapse is very important to provide alternative/combination options to patients and improve the treatment outcomes.

The currently used techniques measuring changes in tumor size alone to assess response to RT are inadequate in shedding light on the underlying biology that drives the response to radiation. Furthermore, analyzing changes in tumor size is a measurable clinical outcome that is seen only towards the end or after the treatment has finished. The patients who fail to respond to treatment initially go undetected contributing to tumor progression and delay in the initiation of an alternative treatment strategy. Non-responding patients will also be at risk of developing RT-induced side effects for no therapeutic gain.

Using a tissue biopsy-based test for performing long-term follow-up to monitor relapse evolution and treatment response in order to reorient treatment regimens in a timely manner will require multiple invasive biopsies which are not always feasible, and they often miss alterations found in sites other than the primary tumor, which influences therapy response and efficacy.

In some cases, the eradication of radioresistant tumors relies on dose escalation or the use of multimodal therapies including combination therapy. FLAME trial, a large-scale study involving 571 patients, indicated that men with localized intermediate- and high-risk PCa receiving an additional boost exhibited 50% less recurrence. A test to stratify PCa patients who can benefit from boost will allow dose escalation in patients whose tumor characteristics can tolerate such without causing side effects.

What is needed is a novel, non-invasive strategy for identifying whether a tumor will be sensitive or resistant to cancer therapy, which patients are suitable for RT and which patients show higher potential for relapse. The monitoring of tumor response during cancer therapy will prevent the continuity of ineffective treatments that are potentially harmful to the patient and can be halted, and other preventive/beneficial treatment measures can be taken based on their tumor characteristics.

### BRIEF SUMMARY

To address the critical unmet need, the inventors have developed a non-invasive/minimally-invasive liquid biopsy biomarker test which can define a patient's eligibility for RT, recommend dose adjustments, monitor the effectiveness of the given dose and identify early relapse. A test which can classify the patients into high, medium, weak responders and nonresponders will help physicians (i) identify those who would benefit from mitigative interventions (e.g., fractionation dose changes); (ii) deliver confidence for dose escalation/dose de-escalation; (iii) provide guidance in determining the tolerable doses in cases of reirradiation; (iv) identify specific molecularly targeted agents (MTAs) which can provide additional benefit to non-responder and/or weak responders and/or patients who stop responding to treatment or develop relapse. Molecular Targeted Agents/therapies/drugs are substances that block the growth and spread of cancer by interfering with specific molecules (“molecular targets”/biomarkers). An example is checkpoint inhibitor(s) which interact with checkpoint protein (PD-L1). Such biomarkers-based testing can advance RT towards accurate radiation precision medicine to improve its outcomes.

The present invention relates to the identification and use of gene expression profiles, signatures, or patterns of expression of a set of biomarker genes with clinical relevance to cancer. In particular, the invention is based on the gene expression analysis of nucleic acids, preferably transcripts of biomarker genes, obtained from exosomes, circulating tumor cells, or nucleic acids isolated from a biological sample. In particular, expression analysis of these biomarker genes is used in providing a Radiation Response Index (RRI) indicative of the tumor's sensitivity or resistance to radiation, allowing the prediction of responsiveness to radiation therapy before, during and post-treatment.

A ‘Before treatment RRI’ or ‘Pre-treatment RRI’ allows classification of patients into different types of responders (strong responder, responder, weak responder and non-responder) and provides guidance/recommendation for dose escalation, de-escalation and combination therapy for patients based on the category of responder Patients who have a pre-treatment score of high radiation sensitive RRI (RS-RRI) (radiation sensitivity (RS) with high sensitivity to RT) will be classified as strong responder (SR) and will be candidates for reduced RT dose. The medium RS-RRI index patients (with medium sensitivity to RT) will be classified as responder (R) and will be subject to the standard RT dose options. The medium radiation resistant patients, RR-RRI (with low sensitivity to RT) will be classified as weak responder (WR) and will be candidates for dose escalation. The low RRI index patients (with very low sensitivity to RT) will be classified as non-responder (NR) and may either be candidates for dose escalation, or candidates for alternate therapies. For the former, if the dose escalation starts showing changes in biomarkers towards increased radio-resistance/metastasis, they can be recommended for alternate therapies or combination therapies based on the presence of specific molecular targets (e.g. epidermal growth factor receptor (EGFR), androgen receptor (AR), and programmed death ligand 1 (PD-L1). When the low pre-treatment RRI index classifies the subject as non-responder and/or weak responder the expression of one or more biomarkers from Table 1 in a tumor sample from said patient can point to specific molecular targeted therapies (MTAs). MTAs can be given prior to radiation, together, post-radiation/sequential/or any other way as seem necessary by clinicians. Expression of biomarker can be determined at nucleic acid/protein level.

In subjects or patients who do not respond to treatment and/or show failure to respond to therapy and after some time develop relapse, use of the biomarkers to identify specific molecularly targeted agents (MTAs) provides additional benefit. By analyzing the expression in a patient's biological sample of one or more biomarkers in Table 1, the most effective treatment based on the characteristic of their individual tumor can be delivered thereby meeting the goal of precision medicine: ensuring the delivery of the right treatment to the right patient at the right time.

During treatment, a ‘Post Treatment RRI’ monitoring allows identification of the development of ongoing resistance and provides options for combination therapies/alternate therapies options based on presence of biomarkers as described above. Post-treatment RRI also facilitates the early identification of relapse, and for patients at high risk of recurrence, a careful watch and implementation of appropriate strategy based on tumor sensitivity can be implied.

Therefore, the above clinical use of RRI provides a very helpful parameter for personalized medicine relating to the diagnosis/prognosis and treatment of cancer patients. The RRI may be used alone or in combination with other means and methods that provide information on the patient's personal/clinical disease status.

In one aspect, a radiation response index (RRI) indicative of response of a tumor to a dose of radiation, wherein said RRI is based on the relative changes in gene expression of one or more genes in a biomarker panel in a biological patient-derived sample due to the radiation as compared to a reference, wherein said biomarker panel is one or more genes numbered 1-16 in Table 1, wherein the RRI is able to classify response of a tumor as resistant or sensitive to radiation therapy.

In another aspect, a method for establishing a Radiation Responsive Index indicative of response of a tumor to a dose of radiation, comprising analyzing differential gene expression products of one or more biomarkers in Table 1 in a biological sample of tumor sensitivity or resistance to radiation, and establishing said Index using an algorithm whose parameters were obtained from a standard or training set.

In yet another aspect, a method for determining whether a patient suffering from a cancer will achieve a response after radiation therapy comprising determining an RRI in a biological liquid sample obtained from the patient before radiation therapy, wherein a patient is classified into strong responder, responder, weak responder, and non-responder, concluding that the patient will not achieve a response with standard RT dose when the RRI classifies the patient as non-responder, concluding that the patient will achieve response with dose-escalation when the RRI classifies the patient into weak responder, concluding that the patient will achieve response with dose de-escalation when the RRI classifies the patient into strong responder, concluding that the patient will achieve response with standard dose when the RRI classifies the patient into responder.

In one aspect the invention provides a method of classifying a patient as a responder (strong responder, responder and weak responder) or non-responder to RT before treatment comprising (a) analyzing a patient derived sample for differential expression of the gene products of one or more genes of Table 1, and (b) classifying the patient from which the sample was derived as a responder or non-responder based on the results of step (a), wherein the classifying is performed by reference or comparison to a standard or a training set or using an algorithm whose parameters were obtained from a standard or training set.

In another aspect, a method for determining whether a patient suffering from cancer will achieve a response after radiation therapy from continued standard RT comprising i) determining the RRI in a first liquid sample obtained from the patient before radiation therapy wherein the RRI can classify a patient into strong responder, responder, weak responder, and non-responder, ii) determining RRI in a second and subsequent liquid sample(s) obtained from the patient during or just after radiation therapy wherein the RRI can classify a patient into strong responder, responder, weak responder, and non-responder, iii) comparing the RRI determined at i) with the RRI determined at ii), and, iv) concluding that patient will achieve a response after continued standard RT when RRI classifies the patient into responder or strong responder.

In another aspect, a method for determining whether a patient suffering from cancer will achieve a response after radiation therapy with combination therapy comprising determining the RRI of a first liquid sample obtained from the patient before radiation therapy wherein the RRI classifies the patient into weak and/or non-responder, administering combination therapy, determining RRI in a second liquid sample based on the presence/absence of a change in expression of one or more biomarkers from Table 1 wherein a change alters the RRI classification of said patient to responder indicates that patient will achieve a response after combination therapy. Combination therapy includes combining RT with hormone therapy, a modulator/molecular targeted therapy, or other factors that may provide an improvement in RT efficacy.

In another aspect, a method for determining whether a patient suffering from cancer will achieve a response after radiation therapy with specific combination therapy comprising determining the RRI of a first liquid sample obtained from the patient before radiation therapy wherein the RRI classifies the patient into weak and/or non-responder. For administering the right combination therapy a selection based on biomarker expression from primary tumor sample using IHC and/or gene expression. Combination therapy includes combining RT with hormone therapy, a modulator/molecular targeted therapy, or other factors that may provide an improvement in RT efficacy.

In another aspect, a method for determining whether a patient suffering from cancer will achieve a response after radiation therapy with specific combination therapy comprising determining the RRI of a first liquid sample obtained from the patient before radiation therapy wherein the RRI classifies the patient into weak and/or non-responder. For administering the right combination therapy, a selection based on biomarker expression from exosomes/liquid biopsy. Combination therapy includes combining RT with hormone therapy, a modulator/molecular targeted therapy, or other factors that may provide an improvement in RT efficacy.

In another aspect, a method when a patient who stopped responding to treatment based on RRI where RRI post-therapy classifies the patient as non-responder/and/or weak responder will achieve a response with specific combination therapy. For administering the right combination therapy, a selection based on biomarker expression (gene, protein, IHC) from exosomes/liquid biopsy or serial tumor biopsy. Combination therapy includes combining RT with hormone therapy, a modulator/molecular targeted therapy, or other factors that may provide an improvement in RT efficacy.

In another aspect, a method for determining whether a patient suffering from a cancer will achieve a response after radiation therapy comprising i) determining the RRI in a first liquid sample obtained from the patient before radiation therapy, ii) determining RRI in a second and subsequent liquid sample(s) obtained from the patient during or just after radiation therapy, iii) comparing the RRI determined at i) with the RRI determined at ii), and, iv) concluding that the patient will achieve response when the RRI in ii) classifies the patient into strong responder, responder and weak responder or concluding that the patient will not achieve a response when the RRI determined at ii) classifies the patient as non-responder.

In another aspect, a method for determining whether a patient suffering from a cancer will achieve a response after radiation therapy after dose escalation and/or boost comprising i) determining the RRI in a first liquid sample obtained from the patient before radiation therapy, ii) determining RRI in a second and subsequent liquid sample(s) obtained from the patient during or just after radiation therapy, iii) comparing the RRI determined at i) with the RRI determined at ii), and, iv) concluding that the patient will require dose escalation and or boost if RRI in ii) classifies the patient into weak responder and/or non-responder.

In another aspect, a method for determining whether a patient suffering from a cancer will achieve a response after radiation therapy after dose de-escalation comprising i) determining the RRI in a first liquid sample obtained from the patient before radiation therapy, ii) determining RRI in a second and subsequent liquid sample(s) obtained from the patient during or just after radiation therapy, iii) comparing the RRI determined at i) with the RRI determined at ii), and iv), concluding that the patient will require dose de-escalation if RRI classifies the patient into strong responder.

In one aspect, a method for detecting recurrence of cancer in a subject, comprising providing a biological sample from the subject previously treated for cancer with RT (alone or in combination), assaying an expression level of one or more biomarkers in Table 1 in the biological sample from the subject and determining an RRI wherein the RRI is able to classify response of a tumor as resistant or sensitive to RT therapy, wherein recurrence of cancer in a subject is detected when RRI classifies response of a tumor as resistant.

In yet another aspect, a method for detecting recurrence of cancer in a subject, comprising providing a biological sample from the subject previously treated for cancer with RT+surgery, e.g. radial prostectomy in the case of PCa, assaying an expression level of one or more biomarkers in Table 1 in the biological sample from the subject wherein the RRI is able to classify response of a tumor as resistant or sensitive to RT+surgery therapy, and wherein recurrence of cancer in a subject is detected when RRI classifies response of a tumor as resistant.

In yet another aspect, a method for detecting recurrence of cancer in a subject, comprising providing a biological sample from the subject previously treated for cancer with RT+ combination therapy, assaying an expression level of one or more biomarkers in Table 1 in the biological sample from the subject and determining an RRI wherein the RRI is able to classify response of a tumor as resistant or sensitive to RT+ combination therapy, and wherein recurrence of cancer in a subject is detected when RRI classifies response of a tumor as resistant.

In another aspect, a method of treating a patient by obtaining an analysis of a patient derived biological sample for differential expression of the gene products of one or more genes of Table 1. The results characterize a patient as a responder (strong responder, responder and weak responder) or non-responder to RT and the characterization step is performed by reference or comparison to a standard or a training set or using an algorithm whose parameters were obtained from a standard or training set.

In another aspect, a method for determining the radiation resistance or sensitivity of a prostate cancer in a subject, comprising measuring a change (increase/decrease) in expression of one or more of the biomarkers in Table 1 using Immunohistochemistry/Immunohistochemical methods on primary tumor (before treatment), and/or serial biopsies during the course of treatment/post-treatment.

The method of the present invention is particularly suitable for discriminating responder from non-responder. As used herein the term “responder” in the context of the present disclosure refers to a patient that will achieve a response, i.e. a patient where the cancer is eradicated, reduced or improved. A non-responder or refractory patient includes patients for whom the cancer does not show reduction or improvement after radiation therapy. When it is concluded that the patient is a non-responder, the physician could take the decision to stop the protocol/change the protocol/use combination/alternate therapy or to avoid any further adverse sides effects RT.

In another aspect, a method for classifying a patient benefitting from alternative/molecular targeted therapy/antibody/and combination therapies, comprising detecting in a biological sample a change in RRI classification to responder resulting from said combination therapy. In one aspect the present invention provides methods to enhance the response of a cancer subject to RT by using one or more modulators by contacting the subject with a modulator prior to, during, simultaneously with, throughout, or following the RT to alter the levels, state, or localization of biomarkers from Table 1 to increase the efficiency of RT. The modulators can be used to enhance the response of a subject to a radiotherapy therapy by increasing the sensitivity/decreasing resistance and altering the RRI and that the likelihood of a subject to respond to a modulator and/or to an anticancer therapy can be predicted from RRI.

In an aspect, a method for identifying whether radiation therapy is beneficial or not to a patient pre-treatment, said method comprising classifying a tumor as sensitive or resistant to radiation prior to radiation exposure, comprising
measuring expression level of at least one nucleic acid sequence from the biomarkers numbered 1-16 in Table 1, in a patient biological sample,
scoring the expression of said at least one nucleic acid sequence based on a Radiation Response Index value (RRI) and classifying the tumor based on the RRI value where a higher RRI correlates with a classification of radiation sensitive tumor and a lower RRI value correlates with a classification of radiation resistant tumor;
   identifying that radiation therapy is beneficial to a patient when the tumor is classified as radiation sensitive, and
identifying that radiation therapy is not beneficial to a patient when the tumor is classified as radiation resistant, non-responding, or weakly responding.

In another aspect, a method for treating a patient with a tumor
wherein the tumor is classified pretreatment as radiation resistant, non-responder, or weak responder, said method comprising
   (i) measuring expression of at least one of the biomarkers numbered 1-16 in Table 1, in a patient biological sample, wherein the biomarker governs a molecular targeted therapy,
   (ii) identifying a variation in expression of the at least one biomarker that indicates a benefit from a molecular targeted therapy, and
   (iii) treating the patient with a regimen that includes the molecular targeted therapy.

In another aspect, a method for treating a patient with a tumor, said method comprising
(i) classifying the tumor as resistant, non-responder, or weak responder to a radiation therapy during a pre-determined regimen, comprising
measuring expression of at least one nucleic acid sequence from the biomarkers numbered 1-16 in Table 1, in a patient sample of exosomes,
scoring the expression levels of said at least one nucleic acid sequence based on a Radiation Response Index value (RRI) and classifying the tumor based on the RRI value where the RRI value correlates with a radiation resistant, non-responder, or weak responder tumor,
(ii) identifying that radiation therapy alone is not beneficial to a patient when the tumor is classified as radiation resistant, nonresponsive or weakly responsive, and
(iii) treating the patient with an alternate therapy regimen, increasing radiation dose, or combining radiation with another therapeutic agent.

In yet another aspect, a method for identifying effectiveness of a radiation treatment post-treatment by identifying tumor response to cancer therapy in a patient after receiving a dosage of radiation therapy, comprising:
measuring expression of at least one nucleic acid sequence from the biomarkers numbered 1-16 in Table 1, in a patient sample of exosomes,
scoring the expression level of said at least one nucleic acid sequence based on an established Radiation Response Index value (RRI) and classifying the tumor based on the RRI value where a higher RRI value correlates with a radiation sensitive tumor and a lower RRI value correlates with a radiation resistant tumor, and
wherein the radiation dosage is effective if the tumor is classified as a radiation sensitive tumor, and
wherein the treatment dosage is ineffective if the tumor is classified as a radiation resistant tumor.

In another aspect, a method for treating a cancer patient receiving a selected radiation therapy regimen comprising measuring the effectiveness of radiation treatment post-treatment by identifying tumor response to cancer therapy in a patient after receiving a dosage of radiation therapy, comprising:
measuring expression of at least one nucleic acid sequence from the biomarkers numbered 1-16 in Table 1, in a patient liquid sample,
scoring the expression level of said at least one nucleic acid sequence based on a Radiation Response Index value (RRI) and classifying the tumor based on the RRI value, capable of predicting radiation sensitivity of a tumor, said index based on the differential expression of the at least one nucleic acid and
wherein the radiation dosage is effective and can be continued if the tumor is classified as a radiation sensitive tumor, and
wherein the treatment dosage is ineffective if the tumor is classified as a radiation resistant tumor, non-responsive, or weakly responsive, and altering the treatment by increasing the dosage, combining the treatment with another treatment agent, or halting the treatment.

In yet another aspect, a method for identifying a cancer patient receiving radiation treatment for a tumor at a high likelihood of relapse post-treatment, comprising
(i) assessing radiation resistance of the tumor by measuring expression of at least one nucleic acid sequence from the biomarkers numbered 1-16 in Table 1 in a patient liquid sample,
scoring the expression level of said at least one nucleic acid sequence based on a Radiation Response Index value (RRI) and classifying the tumor based on the RRI value, capable of predicting radiation sensitivity of a tumor, said index based on the differential expression of the at least one nucleic acid, and
(ii) identifying a patient with a high likelihood of relapse when the tumor is classified as radiation resistant.

In another aspect, a biomarker panel predictive of radiosensitivity of a tumor, said panel comprised of the biomarkers listed in Table 1.

In yet another aspect, a Radiation Response Index (RRI) for predicting radiation sensitivity of a tumor, said index based on the differential expression of at least one biomarker listed in Table 1.

In another aspect, a kit for use in a method for predicting radiosensitivity of a tumor pre-treatment, during treatment, and post-treatment based on a Radiation Response Index, comprising primers and/or probes for determining the expression of at least one nucleic acid sequence from the biomarkers numbered 1-16 in Table 1, and/or reagents for detecting expression of biomarkers in primary tumor samples/exosomes using immunohistochemistry for determining different molecular targeted agent combination therapies, further optionally comprising reagents for isolating exosomes from a liquid biopsy, further comprising reagents for isolating nucleic acids from exosomes, primers and/or probes for determining the gene expression of a reference gene, preferably a housekeeping gene, and optionally further comprising a computer program product, comprising computer readable code stored on a computer readable medium or downloadable from a communications network, which, when run on a computer, implement one or more steps of determining whether a tumor is sensitive or resistant to radiation based on a Radiation Response Index based on the expression of at least one biomarker listed in Table 1, and a system comprising the product of the computer program product. In another aspect the kit includes a PCR kit, a RNA-sequencing kit, or a microarray kit. The product provides an efficient way for obtaining the required expression levels.

### BRIEF DESCRIPTION OF THE DRAWINGS

FIG. 1. Plot showing relative importance of each of the 16 biomarkers in the panel for ΔΔCT.

FIG. 2. Western blot confirming the identity of exosomes isolated from cell culture media of three different prostate cancer cell lines (PC3, LNCaP, 22Rv1) using exosome marker, tetraspanin (CD81). Exosomes from cell culture media were isolated using Qiagen exoEasy kit and western blot analysis was conducted as mentioned in methods.

FIG. 3. Quantification of exosomes from three different prostate cancer cell lines (PC3, LNCaP, 22 Rv1), representing their abundance. Exosomes from cell culture media were isolated using Qiagen exoEasy kit and exosomes quantification was performed using FluoroCet kit from SBI, USA (Catalog #FCET96A-1).

FIG. 4. Quantification of exosomes from three different prostate cancer cell lines (PC3, LNCaP, 22Rv1), representing their abundance. Exosomes from cell culture media were isolated using Ambay's ExoPur™ (Catalog #Catalog #EP-10003) and exosomes quantification was performed using FluoroCet kit.

FIG. 5A-C. Western blot analysis of PD-L1 expression from three different PCa cells lines. Exosomes from cell culture media were isolated using ExoPur™ Dynabeads Mixture from Ambay Immune Sensors and Controls (Catalog #EP-10003) and western blot analysis was conducted as described below. Western blot with CD81 (A) and PD-L1 (B); (C) Quantification of PD-L1 western blot by Image J.

FIG. 6. Cell proliferation and colony formation assay to classify PCa cell lines based on their radio-sensitivity at 2Gy. 0 Gy-Lab: un-irradiated control in lab; 0 Gy: un-irradiated control under travel conditions to the irradiation facility; 2Gy: cells irradiated with 2Gy.

FIG. 7A-B. Distinguishing PCa cell lines for their differences in sensitivities to radiation at 2Gy. A. Example of cell proliferation assay. 0 Gy-Lab: un-irradiated control always kept in the lab; 0 Gy: un-irradiated control that underwent the same travel conditions as the irradiated cells at radiation facility; 2Gy: cells irradiated with 2Gy. B. Survival percent of three different prostate cancer cell lines (PC3, LNCaP, 22Rv1) following 2Gy radiation. 0 Gy-Lab: un-irradiated control always kept in the lab; 0 Gy: un-irradiated control that underwent the same travel conditions as the irradiated cells at radiation facility; 2Gy: cells irradiated with 2Gy.

FIG. 8. Confirmation of real time PCR product from exosomes isolated from cell culture media of prostate cancer cell line LNCaP using exoEasy and ExoPur methods. Exosomes from cell culture media were used to isolate RNA, perform reverse transcription, quantitate synthesized cDNA and perform qPCR. qPCR was performed using 13 ng of single stranded synthesized cDNA. No template control for each biomarker was used to monitor the specificity of reaction.

FIG. 9. Confirmation of real time PCR product for all biomarkers from exosomes isolated from cell culture media of prostate cancer cell line LNCaP. No template control for each biomarker was used to monitor the specificity of reaction.

FIG. 10A-B. Box plot showing change in expression (2−DDCT) through quantitative real time PCR in three different prostate cancer cell lines (22Rv1, LNCaP, PC3). Analysis from a total of 8 independent experiments were used to perform the analysis. Statistical significance (P≤0.05) was established with one-way Anova using statistical software, Minitab. (A). Biomarker (s) showing P value ≤0.05; (B). Biomarker (s) showing P value >0.05.

FIG. 11A-B. Box plot showing MANOVA analysis to evaluate a response to radiation. (A) Biomarker panel distinguishing the radiation response. Total of 52 independent reads from each dose were used to perform the analysis. Comparison of groups was performed by Dunnett test (confidence interval 95%). (B) Biomarker panel distinguished the three cell lines based on their response to radiation. Minimum 34 independent reads of each cell line were used to perform the analysis. Comparison of groups was performed using Games-Howell test (95% confidence interval).

FIG. 12. Box plot showing MANOVA analysis to distinguish the cell lines for their differences in sensitivities to radiation at 2Gy. Biomarker panel distinguished the three cell lines at 2Gy. Minimum 17 independent reads from each cell lines were used to perform the analysis. Comparison of groups was performed using Games-Howell test (95% confidence interval).

FIG. 13. Box plot showing MANOVA analysis to distinguish the three cell lines at 0 Gy while mimicking the differentiation of cell lines similar to 2 Gy. Biomarker panel distinguished the three cell lines at 0 Gy. Minimum 17 independent reads from each cell line were used to perform the analysis. Comparison of groups was performed by Dunnett test (confidence interval 95%).

FIG. 14. Box plot showing MANOVA analysis to distinguish the three cell lines based on relative changes in their expression (2−ΔΔCT). Relative changes in biomarker expression (2−ΔΔCT) values were calculated using GAPDH as the endogenous reference. Biomarker panel distinguished the cell lines into two groups: PC3 in group 1 and 22Rv1 and LNCaP in group 2 as per the Dunnett Test (CI 95%). Minimum 17 independent reads from each cell line were used to perform the analysis.

FIG. 15. Discriminant analysis to classify the cell lines based on their radiation response. Biomarker panel distinguishing the three cell lines based on their relative expression was used for discriminant analysis. Minimum 17 independent reads from each cell line were used to perform the analysis. (A) Radiation Response Index derived from Ensemble Learner Algorithm classifying the samples into radio-resistance and radio-sensitive. (B) Receiver Operator Characteristic Analysis and Area Under the Curve for PC3.

FIG. 16. Box plots showing comparison of responses (delta CT values) of biomarkers at minimal radiation limit 2-4Gy for PC3, a radiation resistant cell line. 8 independent observations per dose were used to perform the analysis. Comparison of groups was performed using Games-Howell test (95% confidence interval).

FIG. 17. Box plots showing comparison of responses (delta CT values) of biomarkers at high radiation limit 8-10Gy for PC3, a radiation resistant cell line. 8 independent observations per dose were used to perform the analysis. Comparison of groups was performed using Games-Howell test (95% confidence interval).

FIG. 18. Box plots showing comparison of responses (delta CT values) of biomarkers at minimal radiation limit 2-4Gy for LNCaP, a medium radiation sensitive cell line, a radiation resistant cell line. 8 independent observations per dose were used to perform the analysis. Comparison of groups was performed using Games-Howell test (95% confidence interval).

FIG. 19. Box plots showing comparison of responses (delta CT values) of biomarkers at medium radiation limit 6Gy for LNCaP, a medium radiation sensitive cell line, a radiation resistant cell line. 8 independent observations per dose were used to perform the analysis. Comparison of groups was performed using Games-Howell test (95% confidence interval).

FIG. 20. Box plots showing comparison of responses (delta CT values) of biomarkers at high radiation limit 8-10Gy for LNCaP, a medium radiation sensitive cell line, a radiation resistant cell line. 8 independent observations per dose were used to perform the analysis. Comparison of groups was performed using Games-Howell test (95% confidence interval).

FIG. 21. Box plots showing comparison of responses of biomarkers at moderate radiation levels (6Gy) for 22Rv1, a high radiation sensitive cell line. Minimum 8 independent observations were used to perform the analysis. Comparison of groups was performed using Games-Howell test (95% confidence interval).

FIG. 22. Box plots showing comparison of responses of biomarkers at high radiation levels (8-10Gy) for 22Rv1, a high radiation sensitive cell line. Minimum 8 independent observations were used to perform the analysis. Comparison of groups was performed using Games-Howell test (95% confidence interval).

FIG. 23. Heatmap showing the involvement of biomarkers at different radiation levels for radiation resistant (PC3), medium sensitive (LNCaP) and highly sensitive (22Rv1) cell line. Eight independent observations per dose were used to perform the analysis for each cell line.

FIG. 24. Western blot confirming the identity of exosomes isolated from human serum samples using exosome marker, tetraspanin (CD81). Exosomes from human serum samples were isolated and western blot analysis was conducted as described in the Examples below.

FIG. 25A-B. Radiation Prediction Profile of Pre-treatment Samples Based on RRI. (A) Exosomes from human serum samples were isolated using methods described below. Radiation Response Index was calculated for each sample. Three independent reads of each sample were evaluated for prediction (Total pre-treatment samples: 34 out of 81, total independent reads 102). Please note the consistency in most cases (the three reads of each sample are grouped together). Threshold RS, resistant to sensitive transition threshold; Threshold MHR, medium to high resistance transition threshold. Threshold MHS, medium to highly sensitive transition threshold. (B) Box plot of the same data shown in A, grouped in terms of High & Medium Sensitivities/Resistances. NR, non-responder (unlikely to respond; WR, weak responder (likely to respond with dose escalation); R, responder (likely to respond to standard dose); SR, strong responder (likely to respond even with dose-de-escalation).

FIG. 26. Example Response Prediction Matrix using the RAD-Senses. Predicted Clinical Response to RT: NR, Non-Responder; WR, Weak Responder; R, Responder; SR, Strong Responder. NR unlikely to respond; WR, likely to respond with dose escalation; R, responder likely to respond to standard dose; SR, strong responder likely to respond even with dose-de-escalation.

FIG. 27. Prediction from pre-treatment based classification can guiding the dose adjustment decisions. Pre-treatment sample predictions to sub-classifying the patients based on additional radiation treatment (Boost). RRI classified both with and without Boost categories into RS (Responder/Strong Responder) and RR (Nonresponder). RT combined with long-term ADT is a standard of care option for men with high-risk and locally advanced PCa. RT/RT+H treatment (without Boost category) categorizes patients into RR (Nonresponder/weak responder) and RS (Responder/Strong Responder).

FIG. 28A-B. Radiation Prediction Profile of Post-treatment Samples Based on RRI. (A) Exosomes from human serum samples were isolated using methods described below. Radiation Response Index was calculated for each sample. Three independent reads of each sample were evaluated for prediction (Total post-treatment samples: 47 out of 81, total independent reads 141). (B) Box plot of the same data shown in A, grouped in terms of High & Medium Sensitivities/Resistances. Threshold RS, resistant to sensitive transition threshold; Threshold MHR, medium to high resistant transition threshold. Threshold MHS, medium to highly sensitive transition threshold.

FIG. 29A-B. Example 1 of RRI-based Prediction for Monitoring of Treatment Response and its Comparison with Clinical Status for Non-responder: Exosomes from human serum samples were isolated as described below. Radiation Response Index was calculated for each sample to categorize them into radiation resistant (high and medium) and radiation sensitive categories (high and medium). Three independent reads were evaluated for each sample. (Total samples representing longitudinal collection: 48 out of 81). To demonstrate proof-of-concept on the functionality of RRI-based prediction for treatment monitoring prediction, the inventors first separated the patient samples with longitudinal collection and analyzed the matching pre-treatment and post-treatment time intervals from the same patient (longitudinal retrospective sample collection). (A) shows the ability of the exosome-based biomarker approach to monitor a shift in molecular processes for the outcome of radiation treatment. (B) shows how RRI monitoring can assist in determining effectiveness of predicting and altering therapy pre-treatment, during treatment, and post-treatment. The RRI based monitoring dose adjustment prediction for a non-responder by a dose escalation/boost matched with clinical outcome.

FIG. 30A-B. Example 2 of RRI-based Prediction for Monitoring of Treatment Response and its Comparison with Clinical Status for Responder: the samples were treated as shown in FIG. 22 above. (A) shows the ability of the exosome-based biomarker approach to monitor a shift in molecular processes for the outcome of radiation treatment. (B) shows how RRI monitoring can assist in determining effectiveness of predicting and altering therapy pre-treatment, during treatment, and post-treatment. The RRI based dose adjustment prediction for a responder by standard dose matched with clinical outcome.

FIG. 31. Comparison of predictions from RRI vs. PSA score. RRI is able to predict relapse of tumor >12 months earlier than the clinical status. PSA indicated a stable tumor before the identification of clinical status at a later point.

FIG. 32A-C. Specificity and sensitivity of RRI vs PSA. Compared to PSA, RRI has a higher specificity, sensitivity, and lower false positive rate in predicting effectiveness of therapy. In addition, using RRI, possibility of relapse can be predicted 9-14 months earlier than PSA.

FIG. 33A-B. Determination of combination/MTAs therapies options for non-responder and weak responder (classified based on RRI before treatment) to provide optimal benefit. Examples of combination therapy biomarker(s) assessment from exosome using gene expression and primary tumor-based testing using IHC.

FIG. 34. Detection of additional biomarkers on primary tumors for combination therapies for weak responder/non-responders using IHC.

FIG. 35. Flow chart of RRI based testing with determination of treatments based on RRI scores from pre-treatment, during treatment, post-treatment/surveillance.

The above-described and other features will be appreciated and understood by those skilled in the art from the following detailed description, drawings, and appended claims.

### DETAILED DESCRIPTION

The inventors have identified a panel of individual biomarkers and biomarker profiles that exhibit differential gene expression. A Radiation Response Index (RRI) was created based on biomarker profiles established from cell lines that are sensitive as compared to cell lines that are resistant to radiation therapy. The RRI allows identification of sensitivity or resistance to radiation in clinical samples from patients with tumors pre-radiation and post-radiation treatment, as well as changes in radiation sensitivity of the tumor pre-treatment, during treatment, and post-treatment. A radiation response index (RRI) based on the expression of the biomarkers in tumor cells or exosomes and the response of cells was calculated and can be used to classify the tumors into different categories: resistant (non-responder), medium resistant/low radiation sensitivity (weak responder), and highly sensitive (responder) or medium radiation sensitivity (medium responder). RRI was calculated separately for Pre-Treatment clinical samples that received no radiation, and for Post-Treatment clinical samples that had been subjected to radiation.

Prostate cancer (PCa) is a heterogeneous disease and the absolute benefit from radiation therapy (RT) is not equal across all risk groups. Biomarkers signatures which can allow personalized treatment by selecting appropriate patients who might, or might not, benefit from RT or whose radiation therapy might be escalated or de-escalated will allow clinicians to tailor therapies according to the molecular characteristics of individual tumors, improve survival rates and reduce toxicity. The predictive RRI biomarker-based test of the present invention is able to categorize PCa patients (Gleason score ≥6) into (a) non-responder (RR) and responder (RS) categories before RT treatment; (b) likelihood of early relapse (RR) and stable (RS) post RT treatment. Categorization of patients before treatment will allow clinicians to develop a radiation treatment (dose escalation, de-escalation, standard) plan based on individual patient's tumor characteristic. RRI-based post therapy monitoring/surveillance of PCa patients will enable the identification of early relapse to implement a quicker and accurate immediate and optimal treatment. Monitoring of patients during RT treatment based on their sensitivities will allow delivery of suitable alternative treatments to high-risk patients and dose escalation to tumors in less sensitive patients during early phases of treatment. The present invention offers additional advantages of exploring the possibility of use of other modalities (e.g., immunotherapy, molecular targeted therapy) in combination with RT for patients who can't get maximum benefit from RT alone and allows the clinician to make a decision to administer a molecular targeted therapy prior to, during, or post radiation therapy.

Therefore, the invention allows for predicting the response of the tumor to radiation prior to treatment and monitoring the response of the tumor during and post-treatment thereby providing the clinician insight to assist in the decision to provide radiation therapy, to increase or decrease the radiation dose during therapy, to anticipate early relapse in a patient, and to provide combination therapy with specific molecular targets prior to, during and post radiation therapy.

Results indicate that the exosome-based biomarker analysis test described herein compares well with clinical status on known samples of prostate cancer, allows the pre-treatment prediction of efficacy of treatment, the monitoring of treatment outcome, and is able to identify relapse even in cases where the PSA level, normally used to identify prostate cancer, was unable to provide accurate information.

The present invention provides, without limitation: 1) methods and compositions for determining whether a cancer therapy and/or a radiation therapy will or will not be effective in stopping or slowing tumor growth and patient treatment; 2) methods and compositions for monitoring the effectiveness of a cancer therapy (radiation therapy agent or a hormone therapy or a combination of agents); 3) methods and compositions for treatments of tumors comprising a cancer therapy and/or a radiation therapy; and 4) methods and compositions for identifying specific therapeutic agents and combinations of therapeutic agents that are effective for the treatment of tumors in specific patients.

The biomarkers of the present invention, whose expression correlates with the response to a therapeutic cancer agent, are identified in Table 1. By examining the expression of one or more of the identified markers or marker sets in a biological sample, including but not limited to, tissue, cells, exosomes from a cancer patient biopsy/body fluids and scoring the expression profile based on the Radiation Response Index, it is possible to determine which therapeutic agent or combination of agents will be most likely to reduce the growth rate of the cancer cells. By examining the expression of one or more of the identified markers or marker sets in a cancer patient sample and scoring the expression profile based on the Radiation Response Index, it is also possible to determine which therapeutic agent or combination of agents will be the least likely to reduce the growth rate of cancer cells. By examining the expression of one or more of the identified markers or marker sets, it is therefore possible to eliminate ineffective or inappropriate therapeutic agents. Importantly, these determinations can be made on a patient-by-patient basis or on an agent by agent basis. Thus, one can determine whether or not a particular therapeutic regimen is likely to benefit a particular patient or type of patient, and/or whether a particular regimen should be continued.

TABLE 1
BIOMARKERS USED TO DESIGN RRI

| Biomarker | Pathway |
|---|---|
| AR | Cell Proliferation/Resistance to Apoptotic Cell Death |
| PI-3 | Cell Proliferation |
| CD9 | Tetraspanin exosome marker |
| EGFR | Cell Growth/Survival |
| CD81 | Tetraspanin exosome marker |
| HIF-1α | Hypoxia |
| CD63 | Tetraspanin exosome marker |
| Integrin | Cell adhesion, proliferation, migration |
| BRCA1 | DNA Repair |
| P21/p27 | DNA Replication/Cell Cycle/Senescence |
| PD-L1 | Immune Surveillance |
| Snail | Metastasis/Immune Surveillance |
| RAD-51 | DNA Repair |
| AKT | Cell Cycle/Proliferation/Apoptosis |
| Bcl2/Bax | Cell Cycle/Proliferation/Apoptosis |
| p53 | DNA Repair/Senescence |

The present invention is directed to methods of identifying and/or selecting a cancer patient who is responsive to a therapeutic regimen. In particular, the methods are directed to identifying or selecting a cancer patient who is responsive to a therapeutic regimen comprising radiation therapy. Also provided are methods of identifying a patient who is non-responsive to such a therapeutic regimen. These methods typically include determining the level of expression of one or more predictive biomarkers in a biological sample. The biological sample can be a bodily fluid. In one aspect, the biological sample is a component of a bodily fluid, for example exosomes from a patient's liquid biopsy (i.e. any non-solid biological tissue, such as blood, serum, urine, plasma, sweat, saliva, semen), and scoring the expression profile based on a Radiation Response Index, and identifying whether a patient is responsive or nonresponsive to radiation therapy based on the RRI score. The biological sample can be a tumor tissue or tumor cells.

Also provided are methods include therapeutic methods which further include the step of beginning, continuing, commencing, stopping, discontinuing or halting a therapy accordingly where a patient's predictive marker RRI score indicates that the patient would respond (sensitive) or not respond (resistant) to the radiation therapeutic regimen. In another aspect, methods are provided for analysis of a pre-treatment patient, i.e. a patient not yet being treated with a radiation therapy and identification and prediction that the patient would not be a responder (i.e. resistant) to the therapeutic agent and such patient should not be treated with the radiation therapy when the patient's marker RRI score indicates that the patient is resistant. Thus, the provided methods of the invention can eliminate ineffective or inappropriate use of radiation therapy regimens.

Additional methods include methods to determine the activity of an agent, the efficacy of an agent, or identify new therapeutic agents or combinations. Such methods include methods to identify an agent useful as a cancer therapy, for treating a cancer, e.g. a prostate cancer or cancer from a solid tumor based on its ability to affect the expression of markers in a marker set of the invention. For example, an agent which alters the level of expression of a marker or markers such that the level approaches what is in the set predictive for responsiveness to radiation therapy of the cancer would be a candidate inhibitor for the cancer.

The present invention is also directed to methods of treating a cancer patient, with a therapeutic regimen, in particular a radiation therapy (e.g., a radiation, alone, or in combination with an additional agent such as a chemotherapeutic agent) and/or a molecular target therapy, e.g. hormone therapy regimen (a hormone agent, alone or in combination with an additional agent), which includes the step of selecting a patient whose predictive biomarker RRI score indicates that the patient will respond to the therapeutic regimen, and treating the patient with the radiation therapy.

Additional methods include selecting patients that are unlikely to experience response upon treatment or will likely experience relapse. Methods are provided for analysis of a post treatment patient, i.e. a patient already treated with a radiation therapy and identification and prediction that the patient would relapse when the patient's biomarker RRI score indicates that the patient is resistant. Thus, the provided methods of the invention can allow for preventive treatments or additional frequent testing. Furthermore, provided are methods for selection of a patient having aggressive disease, resistant to radiotherapy, and more rapid time to progression.

For example, if pre-treatment or post-treatment RRI from an exosome, liquid biopsy, tumor cells, tissue biopsy or another means of obtaining cancer cells determines the expression of biomarkers from Table 1 predicts the subject as non-responder and/or weak responder, expression of biomarkers in Table 1, at nucleic acid or protein level, governing molecular targeted therapies (MTAs) can be used for an indication if and what molecular target therapy would be effective in treating the tumor. MTAs can be given prior to radiation, together, post-radiation/sequential/or any other way as seem necessary by clinicians.

Similarly, if RRI during treatment from an exosome, liquid biopsy, tumor cells, tissue biopsy or another means of obtaining cancer cells determines the expression of biomarkers from Table 1 predicts the subject as non-responder and/or weak responder, expression of biomarkers in Table 1, at nucleic acid or protein level, governing molecular targeted therapies (MTAs) can be used for an indication if and what molecular target therapy would be effective in treating the tumor. MTAs can be given prior to radiation, together, post-radiation/sequential/or any other way as seem necessary by clinicians.

Additional methods include a method to evaluate whether to treat or pay for the treatment of cancer, e.g. cancer from a solid tumor, by reviewing a patient's predictive marker RRI score for responsiveness or non-responsiveness to radiation therapy.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, preferred methods and materials are described herein. The content of all database accession records (Entrez, GenBank, RefSeq) cited throughout this application (including the Tables) are also hereby incorporated by reference. In the case of conflict, the present specification, including definitions, will control.

The articles “a” and “an” are used herein to refer to one or to more than one (i.e. at least one) of the grammatical object of the article. By way of example, “an element” means at least one element and can include more than one element.

As used herein, a “naturally-occurring” refers to a molecule (e.g., RNA, DNA, protein, etc.) that occurs in nature (e.g. encodes a natural protein, a naturally produced protein, etc).

The term “probe” refers to any molecule which is capable of selectively binding to a specifically intended target molecule, for example a marker or biomarker of the invention. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic monomers.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “Radiation Response Index” refers to the combination of the expression values of at least one biomarker into a single data model. The RRI thereby provides significantly improved classification power to predict the radiation responsiveness of the tumor. The biomarkers useful in providing a RRI have been determined as shown in the Examples below using the Ensemble Learner based model. Other models may be developed based on machine learning algorithms such as support vector machine, naïve bayes, neural network, etc. as known to those in the art, or discriminant analysis algorithms based on classical statistics.

A “marker” or “biomarker” is a naturally occurring polymer corresponding to at least one of the nucleic acids or proteins associated with biomarkers listed in any one of Table 1. For example, markers include, without limitation, sequences recognized by the primers set out below, including sense and anti-sense strands of genomic DNA (i.e. including any introns occurring therein), RNA generated by transcription of genomic DNA (i.e. prior to splicing), RNA generated by splicing of RNA transcribed from genomic DNA, and proteins generated by translation of spliced RNA (i.e. including proteins both before and after cleavage of normally cleaved regions such as transmembrane signal sequences). As used herein, a “marker” may also include a cDNA made by reverse transcription of an RNA generated by transcription of genomic DNA (including spliced RNA). A “marker set” is a group of markers, comprising two or more predictive markers of the invention. Markers of the present invention include the predictive markers identified in Table 1; as identified by the title, gene symbol, and/or Entrez gene identifier and include the representative nucleotide and/or protein sequence or fragment thereof which corresponds to the identifier.

A “predictive marker” or “predictive biomarker” as used herein, includes a marker which has been identified as having differential expression in exosomes of tumor cells known to be radiation sensitive or radiation resistant and has been analyzed to identify whether that expression is characteristic of a patient who is responsive in either a positive or negative manner to treatment with a radiation therapy regimen. For example, a predictive marker includes a marker which demonstrates higher expression in a non-responsive patient; alternatively a predictive marker includes a marker which demonstrates higher expression in a responsive patient. Similarly, a predictive marker is intended to include those markers which demonstrate lower expression in a non-responsive patient as well as those markers which demonstrate lower expression in a responsive patient. Thus, as used herein, predictive marker is intended to include each and every one of these possibilities, and further can include each single marker individually as a predictive marker; or alternatively can include one or more, or all of the characteristics collectively when reference is made to “predictive markers” or “predictive marker sets.” A predictive marker set also can be known as a “classifier”.

The “normal” level of expression of a marker is the level of expression of the marker in cells in a similar environment or response situation, in a patient not afflicted with cancer. A normal level of expression of a marker may also refer to the level of expression of a “reference sample”, (e.g., sample from a healthy subjects not having the marker associated disease). A reference sample expression may be comprised of an expression level of one or more markers from a reference database. Alternatively, a “normal” level of expression of a marker is the level of expression of the marker in non-tumor cells in a similar environment or response situation from the same patient that the tumor is derived from.

“Differential expression” of a marker refers to expression of a marker that varies in level across patients. Furthermore, in this invention reference is made to a marker as “differentially expressed” when its expression level is correlated with, or otherwise indicative of, response or non-response to treatment.

As used herein, “informative” expression is intended to refer to the expression level of a differentially expressed predictive marker which corresponds to responsiveness (sensitivity) or non-responsiveness (resistance). The expression level of a marker in a patient is “informative” if it is greater than a reference level by an amount greater than the standard error of the assay employed to assess expression. Alternatively, a marker that is differentially expressed will have typical ranges of expression level that are predictive of responsiveness or non-responsiveness. An informative expression level is a level that falls within the responsive or non-responsive range of expressions. Still further, a set of markers may together be “informative” if the combination of their expression levels either meets or is above or below a pre-determined score for a predictive marker set as determined by methods provided herein.

A given marker may be indicative of both responsive and non-responsive patients; for example, expression of a predictive marker provided herein above a given threshold (e.g., an informative expression level) may be indicative of a responsive patient, as described herein. Expression of that marker below a given threshold (e.g., below an informative level) may be indicative of a non-responsive patient.

The RRI may be calculated for one, two, three, four, or more biomarkers. Increasing the number of biomarkers increases the robustness of the predictions and increases the separation between classifications. While single biomarkers within the panel were able to distinguish between resistant cell lines and sensitive cell lines (for either case: the 0 Gy cell lines and the radiated cell lines), the ability to distinguish on human clinical pre-treatment samples with reasonable accuracy requires a minimum of 2 biomarkers. The only exception was an accurate post-treatment prediction achieved by using PI3 (AUC min 0.89; AUC 0.94; AUC max 0.97; Edge, 0.9).

Any combination of the biomarkers listed in Table 1 can be chosen to calculate the RRI, and their robustness assessed based on the strength of the Area under the Receiver Operating Curve (AUROC), the Confidence Interval of the AUROC, the Classification Edge (defined as the weighted mean of classification margins—where larger numbers essentially indicate how well the classes are separated by the model), all values that can be calculated using programs such as Matlab, Python, R, and others which are publicly available. Practical considerations such as the number of biomarkers employed in terms of processing cost, and the process time can be taken into consideration when selecting the optimum combination for a particular RRI combination. The RRI distinguishing tumors sensitive to radiation (RS) from tumors resistant to radiation (RR) described herein has values spanning −1 to 1, with zero serving as the threshold between the two classes. For example, a RRI below a predetermined threshold (zero) is indicative for radiation resistant tumor, and an RRI above the predetermined threshold is indicative of a radiation sensitive tumor. The assigning of positive values to RS and negative values to RR is totally arbitrary and can equivalently be used in reverse. Similarly for pre-defining a threshold: for example, in the case of Post-Treatment RRIs, the values spanned the full range from −1 to 1 for the samples analyzed, enabling a threshold between medium and high sensitivity at 0.5 and the threshold for medium and high resistance at −0.5, along with the pre-defined threshold of zero between sensitivity and resistance. In the case of the Pre-Treatment RRIs, since the values only spanned about +/−0.85 for the samples analyzed, more appropriate thresholds of 0.4 and −0.4 were defined with medium to high sensitivity at 0.5 and the threshold for medium to high resistance at −0.4. These thresholds can be predetermined and assigned depending on the data analyzed as is known to a person in the art.

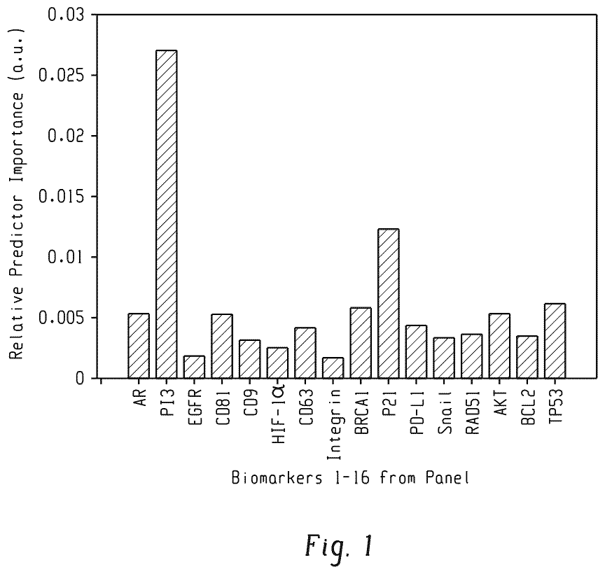

The importance of each biomarker as a predictor when a combination of 16 biomarkers are used is shown in FIG. 1 with PI3 and P21 having the highest relative importance, followed by TP53, BRCA1, AKT, CD81, AR. While all 16 biomarkers listed in Table 1 can be used to determine a RRI, other biomarker combinations chosen from the biomarkers listed in Table 1 can include, for example, an RRI based on 10 biomarkers, such as: AR, EGFR, CD81, BRCA1, P21, PD-L1, Snail, RAD51, AKT, TP53; an RRI based on 6 biomarkers such as: PI3, CD9, HIF-I alpha, CD63, Integrin, BCL2. A particularly preferred combination of biomarker set is AR, HIF-1a, CD63, p21 for pre-treatment assessment of radiation sensitivity and resistance. Another particularly preferred combination is AR, PI3, CD63, p21 for post-treatment assessment of radiation sensitivity and resistance. Other combinations can be as listed below in Tables 2 and Table 3 for calculating pre-treatment and post-treatment RRI, respectively.

As can be seen in Table 2, several combinations of predictive biomarkers serve the purpose of calculating the Pre-treatment RRI ΔCT values of the predictors. A combination of biomarkers {AR,HIF-Iα,CD63,P21}, was chosen to calculate the RRI index, based on the strength of the Area under the ROC curve (AUROC), the Confidence Interval of the AUROC, the Classification Edge (defined as the weighted mean of classification margins—where larger numbers essentially indicate how well the classes are separated by the model; a function in Matlab, edge, calculates this value). While the best AUROC corresponds to this predictor combination, several other biomarker/predictor combinations also serve the purpose of calculating the RRI since they have superior Edge values, despite the loss in AUROC, as shown in the table below—notably, {AR,CD63},{AR,HIF-Iα,CD63}, etc. Practical considerations such as the number of biomarkers employed in terms of processing cost, and the process time also play a role in selecting the optimum combination for a sensor that can measure resistance.

TABLE 2
POSSIBLE BIOMARKER COMBINATIONS FOR
CALCULATING A PRE-TREATMENT RRI

| | AUCmin | AUC | AUCmax | Edge |
|---|---|---|---|---|
| AR, CD63 | 0.77 | 0.85 | 0.91 | 0.50 |
| AR, CD63, Snail | 0.68 | 0.78 | 0.85 | 0.27 |
| CD63, BRCA1 | 0.59 | 0.68 | 0.77 | 0.29 |
| AR, HIF-1α, CD63 | 0.76 | 0.85 | 0.91 | 0.38 |
| AR, CD63, Integrin | 0.70 | 0.78 | 0.86 | 0.31 |
| AR, CD63, P21 | 0.74 | 0.82 | 0.89 | 0.34 |
| AR, CD9, CD63, Integrin | 0.71 | 0.80 | 0.87 | 0.26 |
| AR, CD63, Integrin, P21 | 0.74 | 0.82 | 0.89 | 0.29 |
| AR, HIF-1α, CD63, P21 | 0.83 | 0.90 | 0.95 | 0.32 |
| AR, PI3, HIF-1α, CD63, P21 | 0.52 | 0.61 | 0.71 | 0.25 |
| AR, EGFR, HIF-1α, CD63, P21 | 0.60 | 0.70 | 0.78 | 0.24 |
| AR, CD81, HIF-1α, CD63, P21 | 0.75 | 0.84 | 0.90 | 0.30 |
| AR, HIF-1α, CD63, P21, PD-L1 | 0.50 | 0.60 | 0.70 | 0.21 |
| AR, CD9, HIF-1α, CD63, Integrin | 0.71 | 0.79 | 0.86 | 0.29 |
| AR, CD9, CD63, Integrin, P21 | 0.71 | 0.80 | 0.87 | 0.27 |
| AR, HIF-1α, CD63, P21, AKT | 0.72 | 0.80 | 0.87 | 0.28 |
| AR, HIF-1α, CD63, P21, BCL2 | 0.73 | 0.82 | 0.88 | 0.28 |
| AR, HIF-1α, CD63, P21, TP53 | 0.78 | 0.86 | 0.92 | 0.32 |
| AR, CD9, HIF-1α, CD63, | 0.68 | 0.76 | 0.84 | 0.24 |
| Integrin, P21 | | | | |
| AR, CD63, BRCA1, P21, RAD51 | 0.71 | 0.80 | 0.87 | 0.32 |
| AR, HIF-1α, CD63, Integrin, P21, | 0.81 | 0.88 | 0.93 | 0.26 |
| AKT | | | | |
| AR, CD9, HIF-1α, CD63, | 0.69 | 0.77 | 0.84 | 0.18 |
| Integrin, P21, AKT | | | | |

As can be seen in Table 3, several combinations of predictive biomarkers serve the purpose of calculating the Post-treatment RRI based ΔCT values of the predictors. A combination of {AR,PI3,CD63,P21} was chosen to calculate the RRI index, based on the strength of the AUROC, the Confidence Interval of the AUROC, the instances of relapse misclassifications, and the Classification Edge. While the AUROC corresponding to this predictor combination is 1, several other biomarker/predictor combinations also serve the purpose of calculating the RRI with even superior Edge values—notably, {AR,PI3,CD81}, {AR,PI3,P21}, {PI3,CD81,CD63}, {PI3,CD81,P21}, etc. While combinations of 1PI3,CD811, and {PI3,P21} show high Edge values with a small decrease in AUROC, these are generally not desirable due to the cost of misclassification of relapse, and marked with an asterisk (*). Practical considerations such as the number of biomarkers employed in terms of processing cost, and the process time also play a role in selecting the optimum combination for a sensor that can measure resistance.

TABLE 3
POSSIBLE BIOMARKER COMBINATIONS FOR
CALCULATING A POST-TREATMENT RRI

| | AUCmin | AUC | AUCmax | Edge |
|---|---|---|---|---|
| PI3 | 0.89 | 0.94 | 0.97 | 0.90 |
| AR, PI3 | 0.94 | 0.98 | 0.99 | 0.75 |
| PI3, CD81* | 0.96 | 0.92 | 0.99 | 0.92 |
| PI3, P21* | 0.92 | 0.96 | 0.99 | 0.91 |
| CD81, P21* | 0.60 | 0.68 | 0.75 | 0.32 |
| AR, PI3, CD81 | 1.00 | 1.00 | 1.00 | 0.72 |
| AR, PI3, P21 | 1.00 | 1.00 | 1.00 | 0.71 |
| PI3, CD63, P21 | 0.93 | 0.97 | 0.99 | 0.79 |
| PI3, CD81, CD63 | 0.94 | 0.98 | 0.99 | 0.81 |
| CD81, CD63, P21* | 0.60 | 0.68 | 0.76 | 0.28 |
| PI3, CD81, P21 | 0.93 | 0.97 | 0.99 | 0.87 |
| PI3, CD81, Integrin, P21 | 0.90 | 0.95 | 0.98 | 0.64 |
| PI3, CD81, Integrin, RAD51 | 0.90 | 0.95 | 0.98 | 0.71 |
| PI3, CD81, CD63, P21 | 0.94 | 0.98 | 0.99 | 0.71 |
| PI3, CD81, CD63, P21, TP53 | 0.93 | 0.97 | 0.99 | 0.70 |
| AR, PI3, CD81, P21 | 0.94 | 0.98 | 0.99 | 0.69 |
| AR, PI3, HIF-1α, P21 | 0.94 | 0.98 | 0.99 | 0.69 |
| AR, PI3, CD63, P21 | 1.00 | 1.00 | 1.00 | 0.67 |
| AR, PI3, CD63, P21, Snail | 0.94 | 0.98 | 0.99 | 0.65 |
| AR, PI3, CD9, CD63, P21 | 0.86 | 0.92 | 0.95 | 0.63 |
| AR, PI3, EGFR, CD63, BCL2 | 1.00 | 1.00 | 1.00 | 0.60 |
| AR, PI3, CD81, CD9, P21, TP53* | 0.70 | 0.77 | 0.83 | 0.62 |
| AR, PI3, HIF-1α, CD63, P21, | 1.00 | 1.00 | 1.00 | 0.59 |
| PD-L1, AKT | | | | |
| PI3, CD81, CD9, HIF-1α, CD63, | 0.94 | 0.98 | 0.99 | 0.64 |
| BRCA1, P21, BCL2 | | | | |
| AR, PI3, EGFR, CD81, CD9, | 0.89 | 0.94 | 0.97 | 0.53 |
| CD63, P21, BCL2 | | | | |

The term “EGFR” relates to the human epidermal growth factor receptor, preferably to the sequence as defined in NCBI Reference Sequence NM_005228, more preferably to the nucleotide sequence which corresponds to the sequence of the above indicated NCBI Reference Sequence of the EGFR transcript, and also relates to the corresponding amino acid sequence which corresponds to the protein sequence defined in NCBI Protein Accession Reference Sequence NP_005219.2 encoding the EGFR polypeptide. The term EGFR also relates to the amplicon that can be generated by the primer pair TGA CTA TGT CCC GCC ACT (SEQ ID NO: 1) and TGA TGC AAA TAA AAC CGG ACT G (SEQ ID NO:2).

The term “AR” relates to the human androgen receptor, preferably to the sequence as defined in NCBI Reference Sequence: NM_000044, more preferably to the nucleotide sequence which corresponds to the sequence of the above indicated NCBI Reference Sequence of the AR transcript, and also relates to the corresponding amino acid sequence which corresponds to the protein sequence defined in NCBI Protein Accession Reference Sequence NP_000035.2 encoding the AR polypeptide. The term “AR” also relates to the amplicon that can be generated by the primer pair ACC AAG TTT CTT CAG CTT CCG (SEQ ID NO: 3) and TTG TCC ATC TTG TCG TCT TCG (SEQ ID NO:4).

The term “PI-3” relates to the human peptidase inhibitor, preferably to the sequence as defined in NCBI Reference Sequence: NM_002638, more preferably to the nucleotide sequence which corresponds to the sequence of the above indicated NCBI Reference Sequence of the PI-3K transcript, and also relates to the corresponding amino acid sequence which corresponds to the protein sequence defined in NCBI Protein Accession Reference Sequence NP_002629.1 encoding the PI-3K polypeptide. The term “PI-3K” also relates to the amplicon that can be generated by the primer pair GTC TTG ACC TTT AAC AGG AAC T (SEQ ID NO: 5) and CAA ACA CCT TCC TGA CAC CAT (SEQ ID NO:6).

The term “CD81” relates to the human tetraspanin CD81, preferably to the sequence as defined in NCBI Reference Sequence: NM_004356, more preferably to the nucleotide sequence which corresponds to the sequence of the above indicated NCBI Reference Sequence of the CD-81 transcript, and also relates to the corresponding amino acid sequence which corresponds to the protein sequence defined in NCBI Protein Accession Reference Sequence NP_004347.1 encoding the CD-81 polypeptide. The term “CD-81” also relates to the amplicon that can be generated by the primer pair TCT CCC AGC TCC AGA TAC AG (SEQ ID NO: 7) and GCT CTT CGT CTT CAA TTT CGT C (SEQ ID NO:8).

The term “HIF-1α” relates to the human hypoxia inducible factor 1 subunit alpha (HIF1A), preferably to the sequence as defined in NCBI Reference Sequence: NM_001243084, more preferably to the nucleotide sequence which corresponds to the sequence of the above indicated NCBI Reference Sequence of the HIF-1α transcript, and also relates to the corresponding amino acid sequence which corresponds to the protein sequence defined in NCBI Protein Accession Reference Sequence NP_001230013.1 encoding the HIF-1α polypeptide. The term “HIF-1α” also relates to the amplicon that can be generated by the primer pair CAA CCC AGA CAT ATC CAC CTC (SEQ ID NO: 9) and CTC TGA TCA TCT GAC CAA AAC TCA (SEQ ID NO:10).

The term “CD9” relates to the human tetraspanin 9, CD9, preferably to the sequence as defined in NCBI Reference Sequence: NM_001769, more preferably to the nucleotide sequence which corresponds to the sequence of the above indicated NCBI Reference Sequence of the CD9 transcript, and also relates to the corresponding amino acid sequence which corresponds to the protein sequence defined in NCBI Protein Accession Reference Sequence NP_001760.1 encoding the CD9 polypeptide. The term “CD9” also relates to the amplicon that can be generated by the primer pair GTT TCT TGC TCG AAG ATG CTC (SEQ ID NO: 11) and CAC CAA GTG CAT CAA ATA CCT G (SEQ ID NO:12).

The term “CD63” relates to the human tetraspanin, CD63, preferably to the sequence as defined in NCBI Reference Sequence: NM_001257389, more preferably to the nucleotide sequence which corresponds to the sequence of the above indicated NCBI Reference Sequence of the CD63 transcript, and also relates to the corresponding amino acid sequence which corresponds to the protein sequence defined in NCBI Protein Accession Reference Sequence NP_001244318.1 encoding the CD63 polypeptide. The term “CD63” also relates to the amplicon that can be generated by the primer pairs TTC GGG TAA TTC TCC ATC TGC (SEQ ID NO: 13) and ACT ATT GTC TTA TGA TCA CGT TTG C (SEQ ID NO:14).

The term “Integrin” relates to the human integrin subunit alpha V, preferably to the sequence as defined in NCBI Reference Sequence: NM_001144999, more preferably to the nucleotide sequence which corresponds to the sequence of the above indicated NCBI Reference Sequence of the Integrin transcript, and also relates to the corresponding amino acid sequence which corresponds to the protein sequence defined in NCBI Protein Accession Reference Sequence NP_001138471.2 encoding the Integrin polypeptide. The term “Integrin” also relates to the amplicon that can be generated by the primer pair AAA GTC ATC TAT GCC ATC ACC A (SEQ ID NO: 15) and ACT GCA CAA GCT ATT TTT GAT GAC (SEQ ID NO:16).

The term “BRCA1” relates to the human BRCA1 DNA repair associated (BRCA1), preferably to the sequence as defined in NCBI Reference Sequence: NM_007294, more preferably to the nucleotide sequence which corresponds to the sequence of the above indicated NCBI Reference Sequence of the BRCA1 transcript, and also relates to the corresponding amino acid sequence which corresponds to the protein sequence defined in NCBI Protein Accession Reference Sequence NP_009225.1 encoding the BRCA1 polypeptide. The term “BRCA1” also relates to the amplicon that can be generated by the primer pair ATA CCT GCC TCA GAA TTT CCT C (SEQ ID NO: 17) and AAT GGA AGG AGA GTG CTT GG (SEQ ID NO:18).

The term “P21” relates to the human neuronal vesicle trafficking associated 1; gene synonym P21, preferably to the sequence as defined in NCBI Reference Sequence: NM_001040101 more preferably to the nucleotide sequence which corresponds to the sequence of the above indicated NCBI Reference Sequence of the P21 transcript, and also relates to the corresponding amino acid sequence which corresponds to the protein sequence defined in NCBI Protein Accession Reference Sequence NP_001035190.1 encoding the P21 polypeptide. The term “P21” also relates to the amplicon that can be generated by the primer pair GTC TTG ACC TTT AAC AGG AAC T (SEQ ID NO: 19) and CAA ACA CCT TCC TGA CAC CAT (SEQ ID NO:20).

The term “PD-L1” relates to the human Programmed Death Ligand 1, CD274 molecule (CD274), preferably to the sequence as defined in NCBI Reference Sequence: NM_001267706, more preferably to the nucleotide sequence which corresponds to the sequence of the above indicated NCBI Reference Sequence of the PD-L1 transcript, and also relates to the corresponding amino acid sequence which corresponds to the protein sequence defined in NCBI Protein Accession Reference Sequence NP_001254635.1 encoding the PD-L1 polypeptide. The term “PD-L1” also relates to the amplicon that can be generated by the primer pair CTT CCT CTT GTC ACG CTC AG (SEQ ID NO: 21) and GGC ATC CAA GAT ACA AAC TCA AAG (SEQ ID NO:22).

The term “Snail” relates to the human Snail Family Transcriptional Repressor 1 (SNAI1), preferably to the sequence as defined in NCBI Reference Sequence: NM_005985, more preferably to the nucleotide sequence which corresponds to the sequence of the above indicated NCBI Reference Sequence of the Snail transcript, and also relates to the corresponding amino acid sequence which corresponds to the protein sequence defined in NCBI Protein Accession Reference Sequence NP_005976.2 encoding the Snail polypeptide. The term “Snail” also relates to the amplicon that can be generated by the primer pair GCA CTG GTA CTT CTT GAC ATC T (SEQ ID NO: 23) and GGC TGC TAC AAG GCC AT (SEQ ID NO:24).

The term “RAD-51” relates to the human RAD51 Recombinase (RAD51), preferably to the sequence as defined in NCBI Reference Sequence: NM_001164269, more preferably to the nucleotide sequence which corresponds to the sequence of the above indicated NCBI Reference Sequence of the RAD-51 transcript, and also relates to the corresponding amino acid sequence which corresponds to the protein sequence defined in NCBI Protein Accession Reference Sequence NP_001157741.1 encoding the RAD-51 polypeptide. The term “RAD-51” also relates to the amplicon that can be generated by the primer pair ACA TTA TCC AGG ACA TCA CTG C (SEQ ID NO: 25) and GCC ATG TAC ATT GAC ACT GAG (SEQ ID NO:26).

The term “AKT” relates to the human AKT Serine/Threonine Kinase 1 (AKT1), preferably to the sequence as defined in NCBI Reference Sequence: NM_001014431, more preferably to the nucleotide sequence which corresponds to the sequence of the above indicated NCBI Reference Sequence of the AKT transcript, and also relates to the corresponding amino acid sequence which corresponds to the protein sequence defined in NCBI Protein Accession Reference Sequence NP_001014431.1 encoding the AKT polypeptide. The term “AKT” also relates to the amplicon that can be generated by the primer pair GCG TTC GAT GAC AGT GGT (SEQ ID NO: 27) and CTC CCC TCA ACA ACT TCT CTG (SEQ ID NO:28).

The term “BCL2” relates to the human BCL2 Apoptosis Regulator (BCL2), preferably to the sequence as defined in NCBI Reference Sequence: NM_000657, more preferably to the nucleotide sequence which corresponds to the sequence of the above indicated NCBI Reference Sequence of the BCL2 transcript, and also relates to the corresponding amino acid sequence which corresponds to the protein sequence defined in NCBI Protein Accession Reference Sequence NP000648.2 encoding the BCL2 polypeptide. The term “BCL2” also relates to the amplicon that can be generated by the primer pair AGT CTA CTT CCT CTG TGA TGT TG (SEQ ID NO: 29) and GCT ATA ACT GGA GAG TGC TGA AG (SEQ ID NO:30).

The term “TP53” relates to the human Tumor Protein 53 (TP53), preferably to the sequence as defined in NCBI Reference Sequence: NM_000546, more preferably to the nucleotide sequence which corresponds to the sequence of the above indicated NCBI Reference Sequence of the TP53 transcript, and also relates to the corresponding amino acid sequence which corresponds to the protein sequence defined in NCBI Protein Accession Reference Sequence NP_000537 encoding the TP53 polypeptide. The term “TP53” also relates to the amplicon that can be generated by the primer pair AAT ACT CCA CAC GCA AAT TTC C (SEQ ID NO: 31) and CAA GCA GTC ACA GCA CAT GA (SEQ ID NO:32).

The terms “AR”, “PI-3K”, “EGFR”, “CD81”, “HIF-1α”, “CD63”, “Integrin”, “BRCA1”, “P21”, “PD-L1”, “Snail”, “RAD-51”, “AKT”, “BCL2”, and “TP53” also comprises nucleotide sequences showing a high degree of homology to AR, PI-3K, EGFR, CD81, HIF-1α, CD63, Integrin, BRCA1, P21, PD-L1, Snail, RAD-51, AKT, BCL2, and TP53 respectively, e.g. nucleic acid sequences being at least 75%, 80%, 85%, 90%, 91%, 92%, 93%, 94%, 95%, 96%, 97%, 98% or 99% identical to the sequence as defined in NCBI Reference Sequence, respectively, or amino acid sequences being at least 75%, 80%, 85%, 90%, 91%, 92%, 93%, 94%, 95%, 96%, 97%, 98% or 99% identical to the sequence as defined in NCBI Protein Accession Reference, respectively, or nucleic acid sequences encoding amino acid sequences being at least 75%, 80%, 85%, 90%, 91%, 92%, 93%, 94%, 95%, 96%, 97%, 98% or 99% identical to the sequence as defined in NCBI Protein Accession Reference or amino acid sequences being encoded by nucleic acid sequences being at least 75%, 80%, 85%, 90%, 91%, 92%, 93%, 94%, 95%, 96%, 97%, 98% or 99% identical to the sequence as defined in NCBI Reference Sequence.

A cancer or tumor is treated or diagnosed according to the present methods. “Cancer” or “tumor” is intended to include any neoplastic growth in a patient, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e g, B-cell lymphomas, non-Hodgkins lymphoma). Solid tumors can originate in organs, and include cancers such as bladder, bone, bone marrow, brain, breast, colon, esophagus, gastrointestine, gum, head, kidney, liver, lung, nasopharynx, neck, ovary, prostate, skin, stomach, testis, tongue, or uterus. As used herein, cancer cells, including tumor cells, refer to cells that divide at an abnormal (increased) rate. Cancer cells include, but are not limited to, carcinomas, such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chondosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), and lymphomas (e.g., follicular lymphoma, mantle cell lymphoma, diffuse large Bcell lymphoma, malignant lymphoma, plasmocytoma, reticulum cell sarcoma, or Hodgkins disease); and tumors of the nervous system including glioma, meningioma, medulloblastoma, schwannoma or epidymoma.

A biological sample for use in the present invention can be a bodily fluid. The bodily fluids can be fluids isolated from anywhere in the body of the subject, preferably a peripheral location, including but not limited to, for example, blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid and combinations thereof. For example, the bodily fluid is urine, blood, serum, or cerebrospinal fluid.

In another aspect, the biological sample can be a cancer cell or a cancer cell subpopulation, including but not limited to circulating tumor cells, or cancer stem cells. In one aspect, the cancer stem cell expresses or one more of CD133, CD44, ABCG2, and/or ALDH1A1.

The biological sample can be obtained by any method including blood draw, fine needle aspiration, core biopsy, surgical excision, or other tumor sample acquisition method from a model organism or a subject/cancer patient. The biological sample can be processed in part, or entirely, using one or more manual methods and/or automated systems as is standard in the art.

In one aspect, the biological sample is component of liquid biopsy not limited to exosomes. An exosome is a membrane-derived vesicle released by most eukaryotic cells into extracellular environment which are mainly composed of exosomes (30-150 nm) and microvesicles (200-1000 nm) differing in their cellular origin, abundance and biogenesis. Exosomes are abundantly found in the plasma and malignant effusions derived from cancer patients. Exosomes share certain common characteristics, including shape, size, density, and general protein composition, and mediate effects via transfer of cargo consisting of an array of proteins, selected functional cellular RNAs, and mitochondrial DNA. Any biological sample can be used to isolate exosomes, and can be tested directly, or can be subjected to a processing step before being tested. In one aspect, the sample is a liquid biopsy obtained from a subject, for examples a blood sample, a serum sample, a plasma sample, a semen sample, a sweat sample, a saliva sample, to name a few. Methods for isolating exosomes from liquid biopsy are known in the art and described below. For example, a biological sample can be processed to isolate exosome from blood or any other body fluid by centrifugation/ultracentrifugation, size-based isolation, Dynabeads, immune affinity/affinity-based separation, precipitation such as polyethylene glycol (PEG), chip/microfluidics-based, electric field or other means by using manual methods and/or automated systems. The isolated exosomes can be quantified as described below, for example, using exosome markers tetraspanins (CD9, CD63, CD81) and/or other molecules (Integrins, Alix, TSG101, Rab, HSP70) by ELISA, Western blot, Fluorescence, microscopy, flow cytometry, to name a few.

Methods for isolating nucleic acids from exosomes for analysis of the expression level of one or more biomarkers in Table 1 are also described below, however, other isolation methods known in the art can be used. By ‘nucleic acids’ is meant DNA or RNA. Examples of RNA include messenger RNAs, transfer RNAs, ribosomal RNAs, small RNAs (non-protein-coding RNAs, non-messenger RNAs), microRNAs, piRNAs, exRNAs, snRNAs and snoRNAs. The extracted nucleic acids are further analyzed for the presence, absence, or change in levels of at least one biomarker associated with change due to therapy. For example, analysis of the expression level of a nucleic acid or the presence, absence, or change in levels of at least one biomarker are measured in vivo, ex vivo, or in vitro; and the sample is processed for ex vivo or in vitro analysis using one or more manual methods and/or automated systems.

Analysis of the expression level of a nucleic acid for the presence, absence, or change in levels of at least one biomarker can be measured by PCR. All and different forms of PCR can be used, including but not limited to, qPCR, RT-PCR, endpoint PCR, and Real-time PCR which includes extracting mRNA from the sample, detecting the level of mRNA expression of at least one biomarker in Table 1. Other methods for analyzing the changes in level of biomarkers (increase/decrease) can include using probes, microarrays, NGS sequencing, droplet digital PCR (ddPCR, based on partitioning of the sample into thousands of micro-reactions of defined volume), multiplexed assays, transcription factor identification assays, chromatin immunoprecipitation (CHIP) and CHIP-seq assays, DNA precipitation and DIP-seq assays, microsphere assays, DNAse sensitivity or gel shift assays; the nucleic acid or sequencing/mutation testing includes all forms of sequencing DNA and RNA molecules, whole genome, exome, or specific genes only, including massively parallel signature sequencing (MPSS), 454 pyrosequencing, Illumina™ (Solexa) sequencing, SOLiD™ sequencing, Ion Torrent™ semiconductor sequencing, HeliScope™ single molecule sequencing, single molecule real-time (SMRT) sequencing, sequencing by hybridization, and sequencing with mass spectrometry. Methods for analyzing cell survival, cell proliferation, colony formation/Clonogenic Cell Survival Assays and cell viability are known and include, for example, Hoechst 33342 and propidium iodide (HoPI) assay, water-soluble tetrazolium-based WST-8 assay, and MTT assay for assessing metabolic activity using the tetrazolium dye MTT.

Expression of at least one of the biomarkers in Table 1 can also be measured using immunoassays, for example western blot, dot blot, ELISA, to name a few. Methods of isolating/extracting protein from exosomes to perform western blot for accurate and reliable further analysis are known and described in the Examples below. Analysis of the expression level of the biomarker (presence, absence, or change in levels) of at least one biomarker is performed by densitometry/image J/intensity of western blot band by scanning quantification. Other methods include, but are not limited to, immunohistochemistry, immunocytochemistry, immunofluorescence, as well as multiplexed assays such as flow cytometry, microarrays, or bead-based such as Luminex multiplex assays.

When radiation resistance and sensitivity is determined based on a change in the base level (increase/decrease) in expression of one or more biomarkers in Table 1 using immunohistochemical methods or other methods described herein, biological samples can include samples from primary tumor, and serial biopsies during the course of treatment. A biological sample from a tumor can be obtained as any type of aggregated cells or tumor cells from single or multiple tumor tissues in a given patient. These tumors can be from a human, other mammal, or a xenograft of human cancer cells removed from a non-human mammal (e.g., a mouse). Example cancers that can be tested with the present methods include, but are not limited to, colon cancer, rectal cancer, pancreatic cancer, breast cancer, ovarian cancer, prostate cancer, squamous cell carcinoma, head and neck squamous cell carcinoma, cervical cancer, small cell lung carcinoma, non-small cell lung carcinoma, mesothelioma, kidney cancer, liver cancer, brain cancer, skin cancer, melanoma, bladder cancer, and hematopoietic/blood cancer. The tissue sample can be a portion of a solid tumor or a complete tumor. Such a tissue sample containing tumor cells may be obtained by any method as is known in the art, for example, by taking a biopsy from a patient. Suitable biopsies that may be employed in the present invention include, but are not limited to, blood draws into various tube types to collect blood-based tumors or circulating tumor cells, incisional biopsies, core biopsies, punch biopsies and fine needle aspiration (FNA) biopsies, a core needle biopsy (or core biopsy), Endoscopic biopsy, as well as excisional biopsies. In one aspect, the biomarker expression from Table 1 is the localization, and/or level, and/or state of a molecule, and/or organelle. In some aspects, the molecule being measured is a protein or a nucleic acid; the state of the molecule being measured is phosphorylation, acylation, alkylation, amidation, glypiation, glycation, glycosylation, ubiquitination, degradation product(s), truncation, mutation status, or binding of the molecule(s) to promoters; the localization of the molecule being measured is extracellular or cellular, wherein cellular localization includes intracellular, compartmentalized (e.g. Golgi, endoplasmic reticulum, lysosomal, endosomal, exosomal, mitochondrial, vacuole, cytosolic), nuclear or nucleoli, or membrane (e.g. plasma, nuclear and other organelle membranes) bound; and the state of the organelle being observed is nuclear vacuolation/nuclear autophagy, mitophagy. assigning a semiquantitative score to the observed expression level of biomarker calculated based on (a) intensity of staining ranging from 0 (no staining), 1+ (weak staining), 2+ (distinct staining), 3+ (strong staining) and 4+ (stained cells have minimal light transmission through them, meaning extremely strong/saturated signal) and multiplying by the percent of cells staining at each intensity (0 to 100%). Proportion Score is the proportion of positive tumor cells ranging from 0 to 5 (0=no positive cells, 1=1/100 cells are positive, 2=1/10 cells are positive, 3=1/3 cells are positive, 4=2/3 of cells are positive, 5=all tumor cells are positive). Intensity Score means the average staining intensity of positive tumor cells ranging from 0 to 3 (0=negative, 1=weak, 2=intermediate staining, 3=strong staining). The expression/scoring is achieved using software like image J, Visopharm and other similar software, etc. The method described herein can be used to identify, monitor and predict the existence of inherent RT resistance (before treatment, from primary tissue) and RT acquired resistance (through serial biopsies) during and post RT.

Alternatively or additionally to the reference or normalized expression level of biomarkers set forth in Table 1 as defined herein above, the expression level may also be the expression level of a normalization gene or any other suitable gene or genetic element expressed in a cell e.g. the expression level of a housekeeping gene or the expression level of a combination of housekeeping genes, e.g. GADPH. vinculin, tubulin, actin, ubiquitin, and histones, to name a few. In a preferred aspect, the expression level is determined for a combination of reference genes. The term “normalization gene” refer to any gene which can be used as a reference in a given assay, to allow proper quantification or the biomarker of interest, by comparing said expression levels of a biomarker of interest to the expression level of the normalization gene. Various/normalization biomarkers are available and well known in the art. In other aspects, the comparison is performed using a software classification algorithm. The levels of the biomarkers are evaluated by applying a statistical method such as receiver operating characteristic (ROC) curve cut point analysis, support vector machine, regression analysis, random forests, discriminant analysis, classification tree analysis, OneR, kNN and heuristic naive Bayes analysis, neural nets and other variants.

A cancer is “responsive” or “sensitive” to a therapeutic agent if its rate of growth is inhibited as a result of contact with the therapeutic agent, compared to its growth in the absence of contact with the therapeutic agent. Growth of a cancer can be measured in a variety of ways, for instance, the size of a tumor, imaging or the expression of tumor markers appropriate for that tumor type may be measured. The quality of being sensitive to radiation therapy is a variable one, with different cancers exhibiting different levels of “sensitivity” to a given therapeutic agent, under different conditions. Still further, measures of sensitivity can be assessed using additional criteria beyond growth size of a tumor, including patient quality of life, degree of metastases, etc. In addition, clinical prognostic markers and variables can be assessed (e.g., M protein in myeloma, PSA levels in prostate cancer) in applicable situations.

A cancer is “non-responsive” or “resistant” to a therapeutic agent if its rate of growth is not inhibited, or inhibited to a very low degree, as a result of contact with the therapeutic agent when compared to its growth in the absence of contact with the therapeutic agent. As stated above, growth of a cancer can be measured in a variety of ways, for instance, the size of a tumor or the expression of tumor markers appropriate for that tumor type may be measured. The quality of being non-responsive or resistant to a therapeutic agent is a highly variable one, with different cancers exhibiting different levels of “resistance” to a given therapeutic agent, under different conditions. Still further, measures of resistance can be assessed using additional criteria beyond growth size of a tumor, including patient quality of life, degree of metastases, etc. In addition, clinical prognostic markers and variables can be assessed (e.g., M protein in myeloma, PSA levels in prostate cancer) in applicable situations.

“Treatment” shall mean preventing or inhibiting further tumor growth, as well as causing shrinkage of a tumor. Treatment is also intended to include prevention of metastasis of tumor. A tumor is “inhibited” or “treated” if at least one symptom (as determined by sensitivity/resistance, responsiveness/non-responsiveness, time to progression, or indicators known in the art and described herein) of the cancer or tumor is alleviated, terminated, slowed, minimized, or prevented. Any amelioration of any symptom, physical or otherwise, of a tumor pursuant to treatment using a therapeutic regimen (e.g., radiation therapy regimen or hormone therapy regimen) as further described herein, is within the scope of the invention.

The term “stable cancer” or “stable tumor” means that a sample of an individual does not show parameter values indicating “biochemical recurrence” and/or “clinical recurrence” or “non-progressive cancer state”

The term “relapse” means that a sample of an individual shows parameter values indicating “biochemical recurrence” and/or “clinical recurrence” or “progressive cancer state”.

As used herein, the term “agent” is defined broadly as anything that cancer cells, including tumor cells, may be exposed to in a therapeutic protocol. In the context of the present invention, such agents include, but are not limited to, radiation therapy, molecular therapy agents (MTAs), hormone therapy, as well as chemotherapeutic agents as known in the art and described in further detail herein.

The term “radiation therapy” has its general meaning in the art and refers the treatment of cancer with ionizing radiation. Ionizing radiation deposits energy that injures or destroys cells in the area being treated (the target tissue) by damaging their genetic material, making it difficult for these cells to continue to grow. Different types of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy, in which more than one treatment (fraction) of radiation therapy are given to a subject per day; and hypofractionated radiation therapy, in which larger doses of radiation therapy per fraction is given but fewer fractions.

In some aspects, the method of the present invention is particularly suitable in the context of a hypo fractionated radiation therapy. As used herein the term “hypo fractionated radiation therapy” has its general meaning in the art and refers to radiation therapy in which the total dose of radiation is divided into large doses and treatments are given less than once a day. Typically a treatment course comprises 1, 2, 3, 4, or 5 regimens of ionizing radiation. The ionizing radiation can be combined with the administration of at least one of combination therapy agent affecting the expression of one, any, or all the biomarkers in Table 1.

In one aspect, a method is provided for predicting, determining, or analyzing the response of a subject to combination therapy, RT in combination with another factor or modulator, by analyzing the RRI after administration of the combination therapy. Combination therapy can include administration of a factor or modulator that can alter the expression of any of the biomarkers in Table 1. The modulator can be used to enhance the response of a subject to radiotherapy by increasing the sensitivity, or alternatively decreasing the resistance of the tumor to RT, thereby altering the RRI which can be assessed using the methods and compositions of the invention. Analyzing the RRI, or assessing an alteration in the RRI provides a measure of the likelihood of a subject to respond to a modulator and/or RT. The administration of the combination therapy, or modulator, or additional factor, can be prior to, during, simultaneously with, throughout, or following RT to alter the level, state, or localization of biomarkers from Table 1 to increase the efficiency of RT. One or more modulator(s) can be administered by various means including intravenous, intraperitoneal, intra/transdermal, intratumoral, subcutaneous, inhalation, ocular, sublingual, epidural, vaginal, intranodal, transmucosal, and rectal routes. In one aspect, the cancer subject is administered a pharmaceutically effective amount of the modulator formulated with a pharmaceutically acceptable carrier. In many aspects, the modulator is delivered with a delivery system. The modulator can be one or more of: a cytokine (e.g. an interferon, an interleukin and others), a chemotherapeutic agent, an alkylating agent, a plant alkaloid, an antitumor antibiotic, an antimetabolite, a topoisomerase inhibitor, a molecularly targeted agent, a peptide, an antibody, an oligonucleotide, a DNA damage response repair inhibitors (e.g. double-strand breaks via small interfering DNA (siDNA); a PARP-1 Inhibitors, an ATR Inhibitor, an ATM Inhibitor, a WEE1 Inhibitor, a CDK12 Inhibitor etc.), an anti-DNA antibody, a DNA-PK inhibitor, a nano-particle coated with any/all of above agents, and other modulators known in the art. The combination therapy can include a modulator such as a hormone, for example hormone therapy which includes androgen deprivation therapy (ADT) for prostate cancer. ADT can include administration of a factor or hormone that decreases the amount of androgens in a man's body, and can include luteinizing hormone-releasing hormone (LHRH) agonists, antagonists, androgen inhibitors, antiandrogens, to name a few.

In another aspect, a method for increasing response to radiation in a subject, by destabilizing the expression of specific biomarkers in Table 1 by selective binding using siRNA, thereby altering RRI. In yet another aspect, a method for tumor sensitization, or reducing resistance to RT using compounds designed to enhance the killing effects of radiation (e.g. Enzalutamide, temozolomide, gefitinib, paclitaxel, PARP inhibitors etc) thereby altering RRI.

The term ‘molecular targeted agents’ or ‘MTAs’ are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names.

MTAs can be hormone therapies, slow or stop the growth of hormone-sensitive tumors, which require certain hormones to grow. e.g Androgen receptor (AR) expression in patients with low RRI may contribute to radiation resistance in subject and causes progression. Therapies like Androgen deprivation (ADT) and anti-androgen e.g wherein the anti-androgen compound is abarelix, abiraterone, apalutamide, bicalutamide, degarelix, enzalutamide, flutamide, goserelin, leuprorelin, nilutamide, ozarelix, or a combination of two or more thereof. In aspects, the radionuclide-labeled androgen is a radionuclide-labeled. testosterone, a radionuclide-labeled testosterone analog, a radionuclide-labeled dihydrotestosterone, or a radionuclide-labeled dihydrotestosterone analog. “Androgen” or “androgen compound” refers to testosterone, dihydrotestosterone, androstenedione, dehydroepiandrosterone, androstenediol, androsterone, and the like. In aspects, “androgen” refers to testosterone or dihydrotestosterone. “Anti-androgen compound” refers to any compound that can lower androgen levels in the body. The anti-androgen compounds can be small molecules, peptides, or proteins. In embodiments, the anti-androgen compound refers to a compound used for chemical orchiectomy.

The anti-androgen compound is a gonadotropin-releasing hormone antagonist or a gonadotropin-releasing hormone agonist. In aspects, the anti-androgen compound is a luteinizing hormone-releasing hormone agonist or a luteinizing hormone-releasing hormone antagonist. In embodiments, the anti-androgen compound is abarelix, abiraterone, apalutamide, bicalutamide, degarelix, enzalutamide, flutamide, goserelin, leuprorelin, nilutamide, ozarelix, or a combination of two or more thereof. In embodiments, the anti-androgen compound is abiraterone, In other aspects, the anti-androgen compound is apalutamide. In embodiments, the anti-androgen compound is bicalutamide. In aspects, the anti-androgen compound is enzalutamide. In aspects, the radionuclide-labeled androgen is a radionuclide-labeled testosterone or a radionuclide-labeled testosterone analog. In aspects, the radionuclide-labeled androgen is a radionuclide-labeled dihydrotestosterone or a radionuclide-labeled dihydrotestosterone analog. In aspects, the radionuclide-labeled androgen is a radionuclide-labeled 7α-(E-2′-iodovinyl)-5α-dihydrotestosterone, a radionuclide-labeled 7α-(E-2′-iodovinyl)-17α-methyl-5α-dihydrotestosterone, or a radionuclide-labeled 7α-(E-2′-iodovinyl)-19-nor-5α-dihydrotestosterone. In aspects, the radionuclide is bismuth-21, caesium-131, caesium-137, chromium-51, cobalt-57, cobalt-60, copper-64, copper-67, dysprosium-165, erbium-169, fluorine-18, gallium-67, gallium-68, germanium-68, holmium-166, indium-111, iodine-123, iodine-124, iodine-125, iodine-131, iridium-192, iron-59, krypton-81m, lead-212, lutetium-177, molybdenum-99, palladium-103, phosphorus-32, potassium-42, radium-223, rhenium-186, rhenium-188, rubidium-81, rubidium-82, samarium-153, selenium-75, sodium-24, strontium-82 strontium-89, technetium-99m, thallium-201, xenon-133, ytterbium-169, ytterbium-177, or yttrium-90. In aspects, the radionuclide is iodine-125, iodine-131, or lutetium-177.

In another aspect a strategy may combine ADT with androgen-targeted radionuclide therapy. In another aspect cancer immunotherapy such as sipuleucel-T, AR-directed therapies such as abiraterone acetate (AA) and enzalutamide (Enz), radium-223, and PROSTVAC.

MTAs can be signal transduction inhibitors which block the activities of molecules that participate in signal transduction, the process by which a cell responds to signals from its environment. During this process, once a cell has received a specific signal, the signal is relayed within the cell through a series of biochemical reactions that ultimately produce the appropriate response(s). In some cancers, the malignant cells are stimulated to divide continuously without being prompted to do so by external growth factors. Signal transduction inhibitors interfere with this inappropriate signaling.

Signal transduction molecules (e.g EGFR, AKT, BCL2, PI3 etc.) and pathway governed by expression of molecules from Table 1 in patients with low RRI may contribute to radiation resistance in subject and causes progression. If non-responder and/or weak responder patients show expression of EGFR, use of anti-EGFR antibodies including but not limited Vectibix® (panitumumab), Erbitux® (cetuximab) nimotuzumab, and necitumumab; use of Tyrosine kinase inhibitors (TKIs). In another aspect [0001] In various aspects, MTAs other inhibitors of EGFR, including but not limited to erlotinib, osimertinib, vandetanib, gefitinib and afatinib; an inhibitor of ALK, including but not limited to alectinib, brigatinib, ceritinib, and crizotinib; an inhibitor of one or more members of the VEGF family (VEGF ligand, VEGFR, VEGFR2, VEGFA/B, VEGFR1/2/3), including but not limited to bevacizumab, pazopanib, ramucirumab; sorafenib, Ziv-aflibercept, lenvatinib, axitinib, vandetanib, cabozantinib, and regorafenib; an inhibitor of KIT, including but not limited to axitinib, cabozantinib, imatinib, pazopanib, regorafenib; an inhibitor of HER2, including but not limited to lapatinib, neratinib, pertuzumab, dacomitinib, trastuzumab and ado-trastuzumab emtansine; an inhibitor of CDK4 and CDK6, including but not limited to palbociclib and ribociclib; an inhibitor of BRAF, including but not limited to dabrafenib and vemurafenib; an inhibitor of PARP, including but not limited to niraparib, olaparib, and rucaparib; an inhibitor of one or more members of the JAK family, including but not limited to ruxolitinib and tofacitinib; an inhibitor of mTOR, including but not limited to everolimus and temsirolimus; an inhibitor of MEK, including but not limited to cobimetinib and trametinib; an inhibitor of ERK, including but not limited to ulixertinib; an inhibitor of PDGFR (or part thereof including PDGFRα and PDGFRβ), including but not limited to axitinib, imatinib, olaratumab, pazopanib, regorafenib, and sorafenib; an inhibitor of RAF, including but not limited to regorafenib and sorafenib; an inhibitor of RET, including but not limited to regorafenib, vandetanib and cabozantinib; an inhibitor of MET, including but not limited to cabozantinib and crizotinib; an inhibitor of ROS1, including but not limited to crizotinib; or an inhibitor of any one of the following targets: PIGF, PTCH, Smoothened, RANKL, and B4GALNT1, (e.g. Ziv-aflibercept, vismodegib, sonidegib, denosumab, dinutuximab); an inhibitor of the STAT family, including but not limited to danvatirsen, AZD9150 and TTI-101; an inhibitor of HDAC family (including HDAC3 and HDAC6), including but not limited to nexturastat A, ricolinostat, trichostatin A, vorinostat, panobinostat, vaproic acid, belinostat, and entinostat; an inhibitor of BET family (including BRD1 and BRD4), including but not limited to RG6146, ABBV-075, OTX015/MK-8628, GSK2820151/I-BET151, CC-90010, PLX51107, and LY294002; an inhibitor of NTRK, including but not limited to larotrectinib and entrectinib; an inhibitor of Bcl-2, including but not limited to venetoclax, navitoclax, and obatoclax; an inhibitor of ATM, including but not limited to AZD1390, AZD0156, and M3541; an inhibitor of ATR, including but not limited to AZD6738 and M6620; an inhibitor of A2AR, including but not limited to AZD4635 and CPI-444; an inhibitor of WEE1, including but not limited to adavosertib and ADZ1775; an inhibitor of FGFR, including but not limited to erdafitinib, pemigatinib and TAS-120; or an autophagy influencer. AKT inhibitors including but not limited to Miransertib (ARQ092); BAY1125976, MK-2206, TAS-117, Afuresertib (GSK2110183), Capivasertib (AZD5363), Ipatasertib (GD 0068), Uprosertib (GSK2141795). PI3K inhibitors including but not limited to Piqray, NVP-BEZ235 (BEZ235, Dactolisib), GDC-0084 (RG7666), LY3023414, and other compounds or agents affecting PI3 expression.

MTAs can be Immunotherapies that trigger the immune system to destroy cancer cells. Immune evasion is a hallmark of cancer in which the immune system is unable to mount an effective antitumor response. Non-responder/weak responder to radiation therapy showing low RRI if display expression of molecules such as PD-1 ligand (PD-L1 and PD-L2), Immune Checkpoint Inhibitors which allow to re-engage the immune system to kill tumors by therapy may provide additional benefit. In one aspect, MTAs may include one or more therapeutic PD-1 or PD-L1 antibodies selected from the group consisting pembrolizumab, nivolumab, cemiplimab, atezolizumab, avelumab, and durvalumab pembrolizumab, nivolumab, durvalumab, camrelizumab (SHR1210), sintilimab, tislelizumab, toripalimab, dostarlimab, INCMGA00012, AMP-224, AMP-514, KN035 and CK-301 etc. Immunotherapy can be autologous cellular immunotherapy such as Sipuleucel-T.

MTAs can be Apoptosis inducers cause cancer cells to undergo a process of controlled cell death called apoptosis. Apoptosis is one method the body uses to get rid of unneeded or abnormal cells, but radiation resistance with low RRI cells have developed strategies to avoid apoptosis. Use Apoptosis inducers including but not limited to Actinomycin D, Apoptosis Activator 2, AT 101, BAM 7, Bendamustine hydrochloride, Betulinic acid, Bioymifi Bz 423, C 75, Carboplatin, DNA cross-linking antitumor agent, CFM 4, CARP-1, Chaetoglobosin A, Cisplatin, DNA-alkylating antitumor agent, Cladribine, Deoxyadenosine analog; pro-apoptotic, Cyclophosphamide; Alkylating agent; chemotherapeutic, 2,3-DCPE hydrochloride

Doxorubicin hydrochloride, Antitumor antibiotic agent; induces apoptosis FK 866 hydrochloride, Fludarabine, G5, Gambogic acid; Apoptosis inducer. Activates caspases and inhibits Bcl-2 family proteins; Kaempferol, Methoxyestradiol, Mitomycin C, DNA cross-linking antitumor agent, MPC 6827 hydrochloride, Narciclasine, Oncrasin 1, Oxaliplatin, PCI, histone deacetylase 8 (HDAC8) inhibitor; Piperlongumine, Rifaximin, SMBA 1, Streptozocin, Temozolomide.

MTAs can be Senescence inducers (Sis). Senescence is usually defined as a status of permanent cell cycle arrest. Radiation resistance with low RRI subjects have developed strategies to reduce senescence. SIs can be (1) DNA replication stress inducers (including but not limited to aphidicolin, hydroxyurea, thymidine, bromodeoxyuridine, difluorodeoxycytidine, cyclopentenyl cytosine); (2) DNA-damaging agents, including (2a) DNA topoisomerase inhibitors (including but not limited to doxorubicin, etoposide, daunorubicin, mitoxantrone, camptothecin), (2b) DNA cross-linkers (including but not limited to cisplatin, mitomycin C, busulfan, cyclophosphamide, diaziquone), and (2c) drugs with complex effects (including but not limited to actinomycin D, bleomycin, temozolomide); (3) epigenetic modifiers that inhibit DNA methyltransferases (including but not limited to 5-aza-2′-deoxycytidine), histone deacetylases (including but not limited to sodium butyrate, trichostatin A, MS-275, SAHA, LBH589, phenylbutyric acid, valproic acid), histone acetyltransferases (including but not limited to curcumin, C646), and histone methyltransferases (BRD4770); (4) inhibitors of telomerase activity (including but not limited to SYUIQ-5, BMVC4, pyridostatin, compound 115405, perylene and indole derivatives, harmine, BIBR1532, azidothymidine); (5) inhibitors of cyclin-dependent kinases (including but not limited to palbociclib, roscovitine, ribociclib); (6) activators of p53 (including but not limited to nutlin 3a, FL118); (7) activators of protein kinase C (including but not limited to TPA/PMA, PEP005, PEP008); and (8) reactive oxygen species (ROS) inducers (including but not limited to hydrogen peroxide, tert-butyl hydroperoxide, phenyl-2-pyridyl ketoxime, phenylaminonaphthoquinones, paraquat).

MTAs can be poly (ADP-ribose) polymerase (PARP) inhibitors (PARPi). PARPi are small molecule drugs that target PARP, a crucial enzyme in the repair of single-strand breaks. Radiation resistance with low RRI subjects have developed strategies to repair DNA damage which can be determined with (a) changes in expression of RAD51 and BRCA1 (b) mutations in RAD51 and BRCA. The small molecules and antibodies which target PARPi can provide additional benefits to non-responder and/or weak responder and patients. The antibodies and small molecules include but not limited to rucaparib (Rubraca), olaparib (Lynparza), niraparib (Zejula) and talazoparib (Talzenna), veliparib (ABT-888), 3E10, Fab-F2-iPTD and DIDS, B02, OA-NO2, BRC peptide, CYT-0851, Chicago Sky Blue, Halenaquinone and IBR2, respectively.

MTAs can be agents targeting Snail, by silencing and/or by any other means to change its expression. Radiation resistance with low RRI subjects who have developed strategies to develop metastasis due to EMT transition, the agents affecting the expression of Snail can provide additional benefits to non-responder and/or weak responder patients.

MTAs can be agents targeting TP53. Radiation resistance with low RRI subjects who have developed strategies to develop metastasis due to changes in TP53 expression transition, the agents (e.g. including but not limited to MDM2 inhibitor, gene therapy) affecting the expression of TP53 can provide additional benefits to non-responder and/or weak responder patients.

In another aspect radiation sensitivity and/or resistance is measured by using methods such as colongenic assay, tumor cell proliferation, tumor cell survival and imaging.

The term “prostate cancer” relates to a cancer of the prostate gland in the male reproductive system, which occurs when cells of the prostate mutate and begin to multiply out of control. Typically, prostate cancer is linked to an elevated level of prostate-specific antigen (PSA). In one aspect, of the present invention the term “prostate cancer” relates to a cancer showing PSA levels above 4.0. In another aspect the term relates to cancer showing PSA levels above 2.0. The term “PSA level” refers to the concentration of PSA in the blood in ng/ml.

As utilized herein, by prevent, preventing, or prevention is meant a method of precluding, delaying, averting, obviating, forestalling, stopping, or hindering the onset, incidence, severity, or recurrence of cancer. For example, the disclosed method is considered to be a prevention if there is a reduction or delay in onset, incidence, severity, or recurrence of cancer or one or more symptoms of cancer in a subject susceptible to cancer as compared to control subjects susceptible to cancer that did not receive a treatment or composition described herein. The disclosed method is also considered to be a prevention if there is a reduction or delay in onset, incidence, severity, or recurrence of cancer or relapse or one or more symptoms of cancer in a subject susceptible to cancer after receiving a composition described herein, as compared to the subject's progression prior to receiving treatment. Thus, the reduction or delay in onset, incidence, severity, or recurrence of cancer can be about a 5, 10, 20, 30, 40, 50, 60, 70, 90, 100%, or any amount of reduction in between.

The term “monitoring” as used herein relates to the assessment of a diagnosed or detected cancer, e.g. prostate cancer disease or disorder, e.g. during a treatment procedure or during a certain period of time, typically during 2 months, 3 months, 4 months, 6 months, 1 year, 2 years, 3 years, 5 years, 10 years, or any other period of time. The term “assessment” means that states of disease as defined herein above and, in particular, changes of these states of disease may be detected by comparing the expression level of the at least one biomarker in Table 1 in a sample to a radiation prediction profile based on RRI, in any type of periodical time segment, e.g. every week, every 2 weeks, every month, every 2, 3, 4, 5, 6, 7, 8, 9, 10, 11 or 12 month, every 1.5 year, every 2, 3, 4, 5, 6, 7, 8, 9 or 10 years, during any period of time, e.g. during 2 weeks, 3 weeks, 1, 2, 3, 4, 5, 6, 7, 8, 9, 10, 11, 12 months, 1.5, 2, 3, 4, 5, 6, 7, 8, 9, 10, 15 or 20 years, respectively. The monitoring may also include the detection of the expression of additional genes or genetic elements, e.g. housekeeping genes.

A subject or individual to be diagnosed, monitored or prognosticated prostate cancer relapse according to the present invention is an animal, preferably a mammal, more preferably a human being.

The RRI prediction can be used for all prostate cancer patients with different stages (Gleason score 6 and above) for RT alone, RT+Hormone, RT+Radial prostectomy (Table 4 and Table 5). “Gleason score” or “pGleason” refers to the grading of a sample of prostate cancer by a trained pathologist according to the Gleason system, which assigns a Gleason score using numbers from 1 to 5 based upon similarities in the cells of a sample of prostate tissue to cancerous tissue or normal prostate tissue. Tissue that looks much like normal prostate tissue is given a score or grade of 1 while a tissue that lacks normal features and the cells seem to be spread haphazardly through the prostate is given a score or grade of 5. Scores, or grades of 2 through 4, inclusive, have features in between these possibilities. But because prostate cancers may have areas with different scores or grades, separate scores or grades are given to the two areas that make up most of the tissue. The two scores or grades are added to yield a Gleason score (or Gleason sum) between 2 and 10. As is shown in the Examples below, RRI can predict response to RT for patients with all the Gleason Scores 6, 7 and >7 prostate cancer patients.

TABLE 4
RRI BASED PREDICTION FOR PRE-TREATMENT ACROSS
DIFFERENT STAGES AND GLEASON SCORE

| PCa | | | Treatment | | Response | Treatment |
|---|---|---|---|---|---|---|
| Sample | Stage | Gleason | Status | RRI | Category | Adjustment |
| APP-02 | IV | 4 + 3 = 7 | PRE | (0.5) | Strong | Dose de-escalation |
| | | | | Highly | Responder | can also work |
| | | | | Sensitive | | |
| AMP-23 | II | 4 + 4 = 8 | PRE | (0.33) | Responder | Standard Treatment |
| | | | | Medium | | |
| | | | | Sensitive | | |
| AMP-07 | III | 4 + 3 = 7 | PRE | (−0.34) | Weak | Dose Escalation |
| | | | | Medium | Responder | |
| | | | | Resistant | | |
| ACP-01 | I | 3 + 3 = 6 | PRE | (−0.52) | Non- | Dose Escalation/ |
| | | | | | Responder | Combination/MTAs |

TABLE 5
RRI BASED PREDICTION FOR DURING AND POST-TREATMENT
ACROSS DIFFERENT STAGES AND GLEASON SCORE

| | | | Treatment | | Response | Clinical Status & |
|---|---|---|---|---|---|---|
| PCa Sample | Stage | Gleason | Status | RRI | Category | RRI Comparison |
| APP-02 | IV | 4 + 3 = 7 | POST | (1.0) | Stable | RRI prediction |
| | | | (RT + H) | Highly | | correct when |
| | | | | Sensitive | | tested post-5 months |
| AMP-23 | II | 4 + 4 = 8 | POST | (1.0) | Stable | RRI prediction |
| | | | (RT + H) | Highly | | correct |
| | | | | Sensitive | | |
| ADPC-03 | IV | 3 + 4 = 7 | POST | (−1.0) | Potential | RRI Predicted |
| | | | (RT + H) | Highly | Relapse | correctly 7-8 |
| | | | | Sensitive | | months earlier |
| | | | | | | than critical |
| | | | | | | relapse |
| Radical | IIi | 4 + 3 = 7 | POST | (0.87) | Stable | RRI prediction |
| Prostatectomy | | | (RT) | Highly | | correct when |
| | | | | Sensitive | | tested post-8 month |
| APP-06 | III | 4 + 5 = 9 | POST | (0.80) | Stable | RRI prediction |
| | | | (RT + H) | Highly | | correct when |
| | | | | Sensitive | | tested post-4 month |
| APP-03 | I | 3 + 3 = 6 | POST | (1.0) | Stable | RRI prediction |
| | | | (RT) | Highly | | correct when |
| | | | | Sensitive | | tested post-9 month |
| AMP-22 | III | 4 + 5 = 9 | During | (0.06) | Stable | RRI prediction |
| | | | Treatment | Sensitive | | correct |
| | | | (RT + H) | | | |

In one aspect of the present invention the determination of the presence or amount of marker gene(s) or expression products is accomplished by the measurement of nucleic acid. A nucleic acid molecule of the invention can comprise only a portion of a nucleic acid sequence, wherein the full length nucleic acid sequence comprises a predictive marker of the invention or which encodes a polypeptide corresponding to a marker of the invention. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more predictive markers of the invention. The probe comprises a label group attached thereto, e.g., a radioisotope, a fluorescent compound, an enzyme, or an enzyme co-factor. Such probes can be used as part of a diagnostic test kit for identifying cells or tissues which express the protein, such as by measuring levels of a nucleic acid molecule encoding the protein in a sample of cells from a subject, e.g., detecting mRNA levels or determining whether a gene encoding the protein has been mutated or deleted. Thus, the expression level(s) may be determined by a method involving the detection of an mRNA encoded by the gene.

For example, the measurement of the nucleic acid level of marker gene(s) or expression may be assessed by purification of nucleic acid molecules (e.g. RNA or cDNA) obtained from the sample, followed by hybridization with specific oligonucleotide probes as defined herein above. Comparison of expression levels may be accomplished visually or by means of an appropriate device. Methods for the detection of mRNA or expression products are known to the person skilled in the art.

Alternatively, the nucleic acid level of marker gene(s) or expression may be detected in a DNA array or microarray approach. Typically, sample nucleic acids derived from patients to be tested are processed and labeled, preferably with a fluorescent label. Subsequently, such nucleic acid molecules may be used in a hybridization approach with immobilized capture probes corresponding to the marker genes of the present invention. Suitable means for carrying out microarray analyses are known to the person skilled in the art. Nucleic acid may be detected by antibodies against specific region of DNA/RNA, nanoparticle-based method, etc.

In a standard setup a DNA array or microarray comprises immobilized high-density probes to detect a number of genes. The probes on the array are complementary to one or more parts of the sequence of the marker genes. Typically, cDNAs, PCR products, and oligonucleotides are useful as probes.

A DNA array- or microarray-based detection method typically comprises the following steps: (1) Isolating mRNA from a sample and optionally converting the mRNA to cDNA, and subsequently labeling this RNA or cDNA. Methods for isolating RNA, converting it into cDNA and for labeling nucleic acids are described in manuals for micro array technology. (2) Hybridizing the nucleic acids from step 1 with probes for the marker genes. The nucleic acids from a sample can be labeled with a dye, such as the fluorescent dyes Cy3 (red) or Cy5 (blue). Generally a control sample is labeled with a different dye. (3) Detecting the hybridization of the nucleic acids from the sample with the probes and determining at least qualitatively, and more particularly quantitatively, the amounts of mRNA in the sample for marker genes investigated. The difference in the expression level between sample and control can be estimated based on a difference in the signal intensity. These can be measured and analyzed by appropriate software such as, but not limited to the software provided for example by Affymetrix.

There is no limitation on the number of probes corresponding to the marker genes used, which are spotted on a DNA array. Also, a marker gene can be represented by two or more probes, the probes hybridizing to different parts of a gene. Probes are designed for each selected marker gene. Such a probe is typically an oligonucleotide comprising 5-50 nucleotide residues. Longer DNAs can be synthesized by PCR or chemically. Methods for synthesizing such oligonucleotides and applying them on a substrate are well known in the field of microarrays. Genes other than the marker genes may be also spotted on the DNA array. For example, a probe for a gene whose expression level is not significantly altered may be spotted on the DNA array to normalize assay results or to compare assay results of multiple arrays or different assays.

Alternatively, the nucleic acid level of marker gene(s) or expression may be detected in a quantitative RT-PCR approach, preferably in a real-time PCR approach following the reverse transcription transcripts of interest. Typically, as first step, a transcript is reverse transcribed into a cDNA molecule according to any suitable method known to the person skilled in the art. A quantitative or real-time PCR approach may subsequently be carried out based on a first DNA strand obtained as described above.

Preferably, Taqman or Molecular Beacon probes as principal FRET-based probes of this type may be used for quantitative PCR detection. In both cases, the probes, serve as internal probes which are used in conjunction with a pair of opposing primers that flank the target region of interest, preferably a set of marker gene(s) specific oligonucleotides as defined herein above. Upon amplification of a target segment, the probe may selectively bind to the products at an identifying sequence in between the primer sites, thereby causing increases in FRET signaling relative to increases in target frequency.

Preferably, a Taqman probe to be used for a quantitative PCR approach according to the present invention may comprises a specific oligonucleotide as defined above of about 22 to 30 bases that is labeled on both ends with a FRET pair. Typically, the 5′ end will have a shorter wavelength fluorophore such as fluorescein (e.g. FAM) and the 3′ end is commonly labeled with a longer wavelength fluorescent quencher (e.g. TAMRA) or a non-fluorescent quencher compound (e.g. Black Hole Quencher). It is preferred that the probes to be used for quantitative PCR, in particular probes as defined herein above, have no guanine (G) at the 5′ end adjacent to the reporter dye in order to avoid quenching of the reporter fluorescence after the probe is degraded.

A Molecular Beacon probe to be used for a quantitative PCR approach according to the present invention preferably uses FRET interactions to detect and quantify a PCR product, with each probe having a 5′ fluorescent-labeled end and a 3′ quencher-labeled end. This hairpin or stem-loop configuration of the probe structure comprises preferably a stem with two short self-binding ends and a loop with a long internal target-specific region of about 20 to 30 bases.

Alternative detection mechanisms which may also be employed in the context of the present invention are directed to a probe fabricated with only a loop structure and without a short complementary stem region. An alternative FRET-based approach for quantitative PCR which may also be used in the context of the present invention is based on the use of two hybridization probes that bind to adjacent sites on the target wherein the first probe has a fluorescent donor label at the 3′end and the second probe has a fluorescent acceptor label at its 5′ end.

In an aspect, the gene expression level is determined by an amplification-based method and/or microarray analysis and/or RNA sequencing.

A further aspect of the invention relates to a product comprising:
primers and/or probes for determining the expression level of at least one biomarker listed in Table 1; optionally further comprising primers and/or probes for determining the gene expression level of a reference gene, preferably a housekeeping gene.

In another aspect, it is provided with a nucleic acid array comprising one or more oligonucleotide probes complementary and hybridizable to a coding sequence of at least one biomarker of Table 1, optionally comprising one or more oligonucleotide probes complementary and hybridizable to at least one of the reference genes for determining a Radiation Response-Index as defined herein above.

A “microarray” is a linear or two-dimensional array of discrete regions, each having a defined area, formed on the surface of a generally solid support such as, but not limited to, glass, plastic, or synthetic membrane. The density of the discrete regions on a microarray is determined by the total numbers of immobilized oligonucleotides to be detected on the surface of a single solid phase support, such as at least about 50/cm2, at least about 100/cm2, at least about 500/cm2, but below about 1,000/cm2 in some embodiments. The arrays may contain less than about 500, about 1000, about 1500, about 2000, about 2500, or about 3000 immobilized oligonucleotides in total. As used herein, a DNA microarray is an array of oligonucleotides or oligonucleotides placed on a chip or other surfaces used to hybridize to amplified or cloned oligonucleotides from a sample. Because the position of each particular group of oligonucleotides in the array is known, the identities of a sample oligonucleotides can be determined based on their binding to a particular position in the microarray.

A “oligonucleotide” is a polymeric form of nucleotides, either ribonucleotides or deoxyribonucleotides. This term refers only to the primary structure of the molecule. Thus, this term includes double- and single-stranded DNA and RNA. It also includes known types of modifications including labels known in the art, methylation, “caps”, substitution of one or more of the naturally occurring nucleotides with an analog, and internucleotide modifications such as uncharged linkages (e.g., phosphorothioates, phosphorodithioates, etc.), as well as unmodified forms of the oligonucleotide.

The term “amplify” is used in the broad sense to mean creating an amplification product can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence. It is possible to further use any sequencing method known in the art to identify the sequences.

The term “corresponding” may refer to, where appropriate, a nucleic acid molecule as sharing a substantial amount of sequence identity with another nucleic acid molecule. Substantial amount means at least 95%, usually at least 98% and more usually at least 99%, and sequence identity is determined using the BLAST algorithm, as described in Altschul et al. (1990), J. Mol. Biol. 215:403-410 (using the published default setting, i.e. parameters w=4, t=17). Methods for amplifying mRNA are generally known in the art, and include reverse transcription PCR (RT-PCR) and those described in U.S. patent application Ser. No. 10/062,857 (filed on Oct. 25, 2001), as well as U.S. Provisional Patent Applications 60/298,847 (filed Jun. 2001) and 60/257,801 (filed Dec. 22, 2000), all of which are hereby incorporated by reference in their entireties as if fully set forth. Another method which may be used is quantitative PCR (or Q-PCR). Alternatively, RNA may be directly labeled as the corresponding cDNA by methods known in the art.

Because the invention relies upon the identification of genes (or expressed sequences) that are over- or under-expressed, one aspect of the invention involves determining expression by hybridization of mRNA, or an amplified or cloned version thereof (such as DNA or cDNA), of a sample cell to an oligonucleotide that is unique to a particular gene sequence. Oligonucleotides of this type may contain at least about 20, at least about 22, at least about 24, at least about 26, at least about 28, at least about 30, or at least about 32 consecutive basepairs of a gene sequence that is not found in other gene sequences. The term “about” as used in the previous sentence refers to an increase or decrease of 1 from the stated numerical value. Other embodiments may use oligonucleotides of at least or about 50, at least or about 100, at least about or 150, at least or about 200, at least or about 250, at least or about 300, at least or about 350, or at least or about 400 basepairs of a gene sequence that is not found in other gene sequences. The term “about” as used in the preceding sentence refers to an increase or decrease of 10% from the stated numerical value. Such oligonucleotides may also be referred to as oligonucleotide probes that are capable of hybridizing to sequences of the genes, or unique portions thereof, described herein. In many cases, the hybridization conditions are stringent conditions of about 30% v/v to about 50% formamide and from about 0.01M to about 0.15M salt for hybridization and from about 0.01M to about 0.15M salt for wash conditions at about 55 to about 65° C. or higher, or conditions equivalent thereto.

In other embodiments, oligonucleotide probes for use in the invention may have about or 95%, about or 96%, about or 97%, about or 98%, or about or 99% identity with the marker gene sequences the expression of which shall be determined. Identity is determined using the BLAST algorithm, as described above. These probes may also be described on the basis of the ability to hybridize to expressed marker genes used in methods of the invention under stringent conditions as described above or conditions equivalent thereto.

In another aspect, gene expression analyses on RNA can be performed from RNA isolated from formaldehyde-fixed, paraffin-embedded (FFPE) tissues.

In many cases, the sequences are those of mRNA encoded by the marker genes, the corresponding cDNA to such mRNAs, and/or amplified versions of such sequences. In some embodiments of the invention, the oligonucleotide probes are immobilized on an array, other devices, or in individual spots that localize the probes.

Suitable labels that can be used according to the invention, include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means. The term “support” refers to conventional supports such as beads, particles, dipsticks, fibers, filters, membranes and silane or silicate supports such as glass slides.

In some embodiments, it is provided with a kit for diagnosing, monitoring or prognosticating cancer relapse for identifying an individual for eligibility for prostate cancer radiation therapy comprising: a) an array comprising one or more oligonucleotide probes complementary and hybridizable to a coding sequence of at least one biomarker in Table 1, optionally comprising one or more oligonucleotide probes complementary and hybridizable to at least one of the reference genes, for determining a Radiation Response Index as defined in any of the preceding items, b) a kit control; and c) optionally instructions for use.

Typically, the diagnostic kit of the present invention contains one or more agents allowing the specific detection of marker gene(s) as defined herein above. The agents or ingredients of a diagnostic kit may, according to the present invention, be comprised in one or more containers or separate entities. The nature of the agents is determined by the method of detection for which the kit is intended.

Furthermore, the kit may comprise an amount of a known nucleic acid molecule, which can be used for a calibration of the kit or as an internal control. Typically, a diagnostic kit for the detection of marker gene(s) expression products may comprise accessory ingredients like a PCR buffers, dNTPs, a polymerase, ions like bivalent cations or monovalent cations, hybridization solutions etc. Such ingredients are known to the person skilled in the art and may vary depending on the detection method carried out. Additionally, the kit may comprise an instruction leaflet and/or may provide information as to the relevance of the obtained results.

An additional aspect of the invention relates to a device for performing a method as described herein, comprising: a) a database including records comprising reference gene expression values associated with prostate cancer progression states, each reference profile comprising the expression levels of at least one biomarker in Table 1, and/or b) a user interface capable of receiving and/or inputting a selection of gene expression values of a set of genes, the set comprising the expression levels of at least one biomarker in Table 1, for use in comparing to the gene reference expression profiles or Radiation Response Index in the database; c) an output that displays a prediction of the cancer status according to the expression levels of the set of genes.

A further aspect relates to a computer implemented method for diagnosing, monitoring or prognosticating prostate cancer or the progression state of prostate cancer, comprising the method steps as described herein.

In the context of the present application, the expression “computer implemented method for diagnosing, monitoring or prognosticating prostate cancer or the progression state of prostate cancer,” refers to a method wherein software algorithms classify the sample based on raw data obtained upon measurement of the gene expression level of the genes referred to herein, calculate an RRI score, and based thereon provide a prediction for the patient that is analyzed.

The invention is further illustrated by the following non-limiting examples.

The following Materials and Methods were used in the Examples that follow.

Isolating Exosomes from Human Tumor Cell Lines

Cell lines, LNCaP clone FGC (ATCC® CRL-1740™) and 22Rv1 (ATCC® CRL-2505™) were maintained in RPMI-1640, while cell line PC-3 (ATCC® CRL-1435™) was maintained in F-12 media supplemented with 10% fetal bovine serum, 100U/ml penicillin, 100 mg/ml streptomycin and incubated at 37° C. in 5% CO2. To isolate exosomes from cell culture media (CCM), the cells were first washed with PBS 3 times after 24 hours of seeding the cells, and the media was changed to either RPMI-1640 or F-12 media supplemented with exosome depleted serum from Thermo Fisher (Catalog #A2720801) containing 100U/ml penicillin and 100 mg/ml streptomycin. The cells were incubated further at 37° C. in 5% CO2, and conditioned media with exosomes was collected after 48 hours. Different amounts of condition media from varying cell seeding density were tested to quantify the exosomes. The cells seeded at a density of 2×10⁵ were found to be optimum to perform the collection of CCM for isolation of exosomes. The seeding density of 2×10⁵ from three cell lines showed the following exosome abundances: PC3, 2.37×10⁸; LNCaP, 1.81×10⁸; 22Rv1, 1.86×10⁸.

The isolation of exosomes by the exoEasy™ kit (QIAGEN, Germantown, MD) was done as per the manufacturer's instructions, using a total elution volume of 800 μl; this resulted in the dilution of samples and required an additional concentration step for processing of samples for the proposed downstream applications (western blots, qPCR). To overcome this technical barrier, we used ExoPur™ kit from Ambay Immune Sensors and Controls, (AISC) Hagerstown, MD) to isolate exosomes. The results obtained with ExoPur™ were comparable with Qiagen's exoEasy kit.

Isolating Nucleic Acid from Exosomes from Cells Irradiated with 2Gy for QPCR Analysis

Multiple flasks for each cell line were pooled together to set up the experiments. Briefly, cells were seeded at a density of 2×10⁵ and were washed with PBS 3 times after 24 hours, followed by changing the media to either RPMI-1640 or F-12 media supplemented with exosome depleted serum one shot from Thermo Fisher (Catalog #A2720801) containing 100U/ml penicillin 100 mg/ml streptomycin. The following day, the cells were irradiated with 2Gy. An un-irradiated control that underwent the same travel conditions as the one for irradiation of cells was kept as control. We used a minimum of 3 flasks for each cell line to collect the condition media and isolated the exosomes.

The TaqMan primers to perform qPCR for all biomarkers were synthesized by Integrated DNA Technology, USA. The qPCR reactions for all the biomarkers from un-irradiated and irradiated samples were performed. Briefly, RNA from the exosomes were isolated using Total Exosome RNA and Protein Isolation kit from Invitrogen, USA (Catalog #4478545). cDNA synthesis of eluted RNA was performed using SuperScript™ IV VILO™ Master Mix with ezDNAse™ Enzyme from Thermo Fisher (Catalog #11766050). The quantification of single stranded cDNA was done using the Promega kit (Catalog #E3190). Real time PCR was performed using TaqMan Universal PCR master mix from ABI (Catalog #4304437) and the ABI 7900 HT Real Time PCR machine. No-template control was added for each primer to ascertain the specificity of the reaction. The size and specificity of the qPCR product for each biomarker were previously confirmed by running the PCR product on 1.5% gel. The relative changes in expression of biomarkers were calculated using 2^{-ΔΔCT} method. The endogenous reference, GAPDH was used as control. Statistical significance (P<0.05) was established with one-way Anova using the statistical software package, Minitab.

Isolation of Exosomes from Human Clinical Samples (Serum and Plasma) and Analysis of Clinical Samples.

Exosomes from PCa patient were isolated using 500 μl of plasma/serum samples. Briefly, (i) Isolation of Exosomes: Exosome from 500 μl of plasma and serum were isolated using Invitrogen kits (Catalog #4478360 and 4484450) respectively. (ii) Isolation and Quantification of RNA from Exosomes: RNA was extracted from the exosomes using ‘Total Exosome RNA and Protein isolation’ kit from Invitrogen, USA (Catalog #4478545) as per Ambay SOP (#ER3331). Briefly, RNA extraction was done using, Acid-phenol:chloroform and purification was performed by binding the RNA on glass-fiber filters. Eluted RNA was quantified using a Quanti Fluor RNA Promega kit (catalog #3311) using Perkin Victor Wallace plate reader. Since the purity of nucleic acids extracted from individual samples can strongly affect correctness and repeatability of RT-qPCR quantification therefore we used an in vitro synthesized Luciferase gene RNA (gene absent in human) as an exogenous control and RNA Spike In to verify the parameters of the workflow. (iii) G-Block synthesis: Double-stranded DNA molecules, gBlock of Luciferase were designed and synthesized from Integrated DNA Technologies Inc, USA. Synthesized gBlock was resuspended in RNase/DNase-free distilled water (Invitrogen, Catalog #10977) to a concentration of 20 ng/μl and heated at 50° C. for 20 min as per manufacturer's instructions. Samples were stored at −20° C. until further use. (iv) In vitro RNA Synthesis: RNA transcription was performed by the In-vitro transcription using T7 polymerase kit from New England Biolabs, Inc (Catalog #E2050) according to manufacturer's instructions. In vitro synthesized RNA was purified using Sephadex column from Roche (Cat #11274015001), quantified using RNA Quanti Flour Promega kit (Catalog #3311) and aliquoted for further use in −80° C. AEPC RNA (1.3×10⁴ copies) was added at the time of RNA isolation from the exosomes. (v) Reverse Transcription, cDNA Purification and Quantification: We used high performance SuperScript IV One-Step RT-PCR System from InVitrogen (Catalog #12594025) (sensitivity 0.01 pg RNA, target length up to 13.8 kb), to synthesize cDNA from RNA (10 ng) isolated from exosome. Synthesized cDNA was purified using One Step PCR inhibitor removal kit from Zymo Research (Cat #D6030) and purified single stranded (SS) cDNA was quantified using Quantifluor ssDNA system kit from Promega (Cat #E3190) as per manufacture's instruction. No reverse transcriptase enzyme reaction for each sample was used as control to rule out the possibility of PCR amplification from genomic DNA contamination. (vi) Primers for Pre-amplification and Real Time PCR: All Taqman primers and probes of biomarkers for pre-amplification and real time PCR for biomarkers were synthesized by Integrated DNA Technology, USA. (vii) Pre-Amplification of exosome cDNA: Target-specific preamplification was performed by developing a custom Taqman primer pool containing the primers of Biomarker panel. The cDNA preamplification was performed using TaqMan® PreAmp Master Mix (Life Technologies, Carlsbad, CA, Catalog #) as per manufacture's instruction using custom forward and reverse primers of Biomarker panel (55 nM final concentration) for each target gene, including the Luciferase Spike In. 8 ng of purified cDNA was used for pre-amplification and the reactions were assembled, placed in a Biometra thermocycler and incubated under the following conditions: 95° C. for 10 min followed by 14 cycles of 95° C. for 15 sec and 60° C. for 4 min, enzyme inactivation 99° C. for 10 minutes. Pre-amplified cDNA was diluted to 1:20 before performing qPCR as per manufacture's instruction without introducing amplification bias. (viii) Real Time PCR: The quantitative real time PCR reaction was performed by preparing a reaction mixture in a total 10 μl volume, containing 1:20 pre-amplified product with TaqMan assay, 2×TaqMan Fast Advanced Master Mix from Invitrogen (Catalog #4444965), 20 pmol of forward and reverse primers, 10 pmol of TaqMan probe for each gene/biomarker in separate reactions. All amplifications and detections were carried out in a MicroAmp optical 384-well reaction plate with optical cover (Applied Biosystems). No rt were used as controls to ascertain the specificity of the reaction. GAPDH and Luciferase were used as endogenous and exogenous controls, respectively. Each sample will be run in triplicates. The changes in expression of biomarkers were calculated by delta Ct and 2_{-ΔΔCT} method. Minimum of 10 independent samples will be tested in triplicates each time. Spearman correlation (R₂) of RRI from two different sample type will establish the sample type interchangeability. No template controls (NTC) were processed with each batch of samples as a check for contamination.

Exosome Quantification: The quantification of isolated exosomes was performed by using FluoroCet™ Kit (SBI, System Biosciences, CA, USA). FluoroCet measures the activity of acetecylcholinesterase (AChE), a known exosome protein, by fluorescence emission at 590-600 nm. Isolated exosomes were quantified as per manufacture's instruction.

Immuno-chemistry of primary tumors: Immuno-histochemistry from formaldehyde and embedded in paraffin (FFPE) were performed following manufacture's instruction for primary antibodies. Primary antibodies PD-L1 (E1L3NC)) XP® Rabbit mAb, Androgen Receptor (D6F11) XP® Rabbit mAb, HIF-1α (D1S7W) XP® Rabbit mAb, BRCA1 Antibody, Rad51 (D4B10) Rabbit mAb, p53 (7F5) Rabbit mAb, p21 Waf1/Cip1 (12D1) Rabbit mAb, Akt (pan) (11E7) Rabbit mAb, Snail (C15D3) Rabbit mAb, Bcl-xL (54H6) Rabbit mAb, EGF Receptor (D38B1) XP® Rabbit mAb were purchased from Cell Signaling (MA, USA).

Western Blot: Samples were separated using NuPAGE™ 4 to 12%, Bis-Tris gels (from ThermoFisher, CA, USA) and transferred to Immobilon-P membranes (Millipore, MA). Membranes were blocked for 60 minutes at room temperature in Tris-buffered saline and BSA (Sigma) followed by incubation with primary antibody 1-3 hours at room temperature or overnight at 4 C. Bound antibodies were visualized by horse-radish-peroxidase conjugated secondary antibodies and using Amplified Opti-4CN Substrate Kit from BioRAD (CA, USA) as per manufacture's instruction.

Cell Proliferation Assay: Cell proliferation assays was used to evaluate the effect of radiation on changing the status of live and viable cells. These assays are based on using the water-soluble tetrazolium-based WST-8 assay kit (Dojindo Molecular Technologies, Inc., MD) and can be added directly to cell culture plates without requiring additional processing. Briefly, after irradiation, cells were incubated for an additional 48 hours, and WST-8 assay were performed as per manufacture's instruction. The amount of formazan dye, generated by the activities of dehydrogenases in cells, is directly proportional to the number of living cells, and measured by absorbance at 450 nm. Survival % was calculated by (As−Ab/Ac−Ab)×100 (As: sample absorbance; Ab: Absorbance blank; Ac: Absorbance negative control.

Clonogenic Survival Assay: Prostate cancer cell lines were seeded in 6 well plates and irradiated at 2 Gy. After 14 days of colony growth, medium was removed, and cells washed once in 1 ml of PBS. Colonies were fixed with 700 μL of 3:1 methanol to glacial acetic acid for 5 minutes. Fixative agent was removed and wells air-dried completely prior to staining. Cells were stained for 30 minutes in 500 μL of 1.0% methylene blue (Sigma-Aldrich, USA) in 50% ethanol. Colonies were counted and percentage cell survival was determined as the number of colonies post-treatment relative to the number of colonies within the corresponding 0 Gy control.

### Data Analysis to Calculate Radiation Response Index (RRI)

Part I: 0 Gy and 2Gy data: We used the delta-delta CT values of biomarkers segregated through Eigen Analysis as inputs into classical classification methods such as Logistic regression, and also machine learning algorithms, for classification of the cell lines representing radiation resistance (RR) and radiation sensitivity (RS). The machine learning algorithms {such as k-nearest neighbors, Naive Bayes, Support Vector Machine, Discriminant analysis, Neural Network and Ensemble Learners (result of boosting 100 classification trees)} were run using scripts in Matlab, to iteratively determine the best combination(s) of the set of biomarkers. K-fold partitioning of the data was done during the model development, in order to avoid overfitting. We used CV (Cross Validation) Errors and AUC (Area Under the Curve) for ROC (Receiver Operating Curve) as metrics to assess the performance of each model developed, and the best was adopted to generate an RRI (Radiation Response Index). Thus, the classification algorithms used the relative changes in the expression (2^{-ΔΔCT}) of the cell lines as predictors, and (RR)/(RS) as the response.

Part II 0 Gy versus 2-10Gy: For this dataset, delta CT values were used as inputs into the classification schemes described in Part I, but two separate models were utilized for separating RR and RS cell lines due to the finer distinction afforded by using biomarker combinations that are unique to each model. The first model (Pre Treatment) was for the 0 Gy cell lines that were not subject to radiation, while the second (Post Treatment) was for the cell lines subject to 2-10Gy radiation. These models predicted the classes without error, and were then applied to predict the RR and RS of human clinical samples, segregated as Pre Treatment and Post Treatment. Once Pre Treatment and Post Treatment RRIs for the clinical samples were calculated, these were separated into four groups. In the case of Post Treatment RRIs, the values spanned the full range from −1 to 1 for the samples analyzed, enabling a threshold between medium and high sensitivity at 0.5 and the threshold for medium and high resistance at −0.5, along with the pre-defined threshold of zero between sensitivity and resistance. For the Pre Treatment RRIs, since the values only spanned about +/−0.85 for the samples analyzed, thresholds of 0.4 and −0.4 were more appropriate. These thresholds will be refined as more samples are analyzed, but are not expected to change drastically, based on the longitudinal sample collections reviewed so far, and the clinical assessments thereof.

### Example 1: Creating a Platform for a Simple Non-Invasive Testing Assay Using Exosomes

Develop the exosome assay system for monitoring changes in biomarkers profile. An in vitro model of radiation response derived from lymph node metastasis (LNCaP), primary tumor (22Rv1) and bone metastatic disease (PC-3) from prostate (PCa) was used. Exosome isolation conditions from cell culture media (CCM) of 3 PCa cell lines (LNCaP, 22 Rv1 and PC3) were optimized. Three selected PCa cell lines show differential basal PD-L1 expression (PC3, high, LNCaP, low; 22Rv1 very low/none).

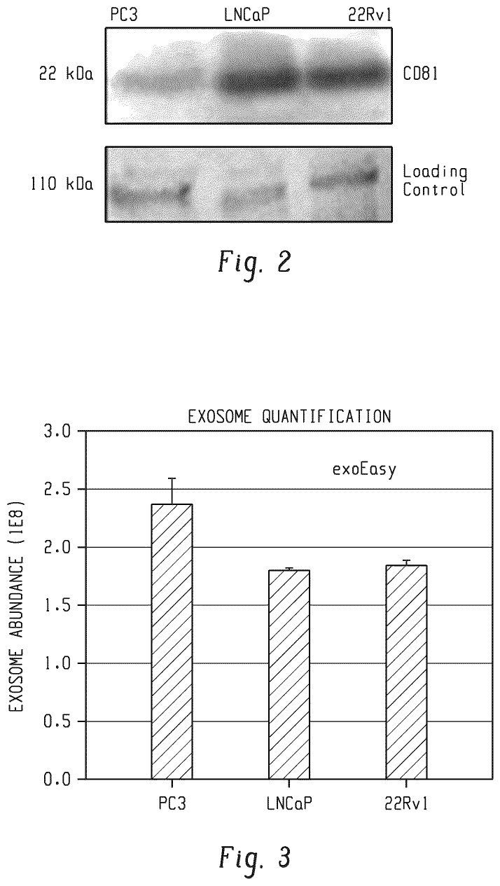

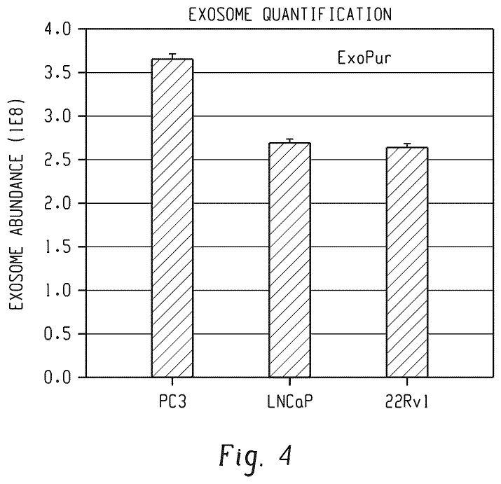

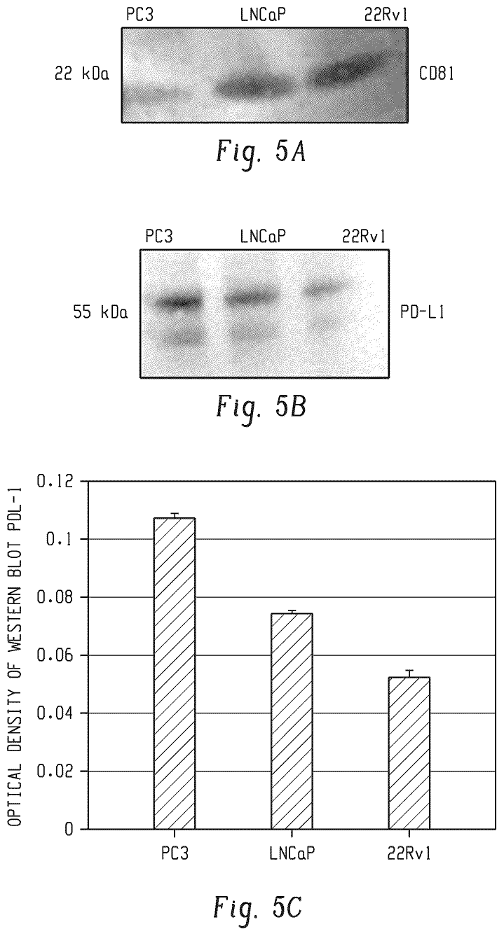

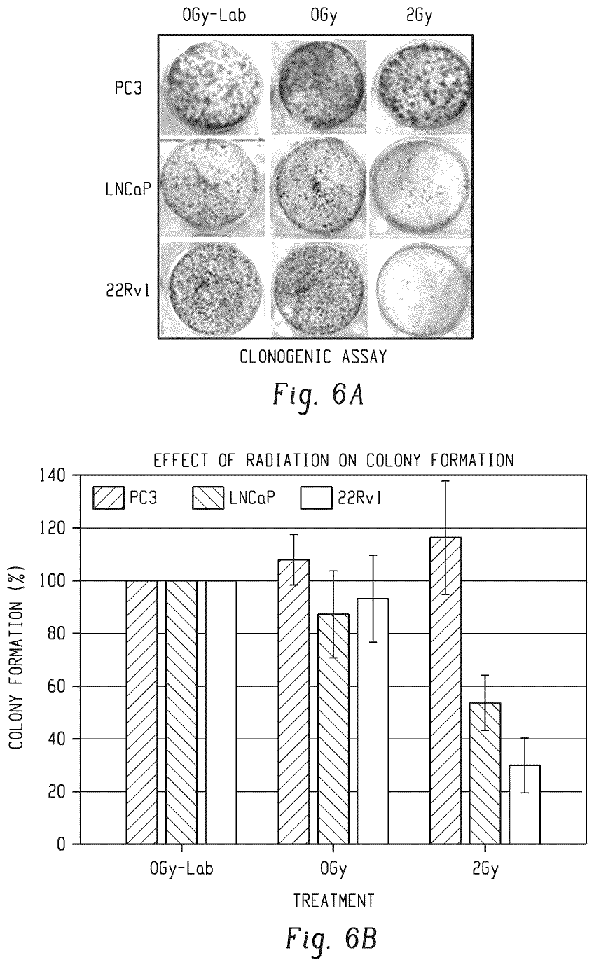

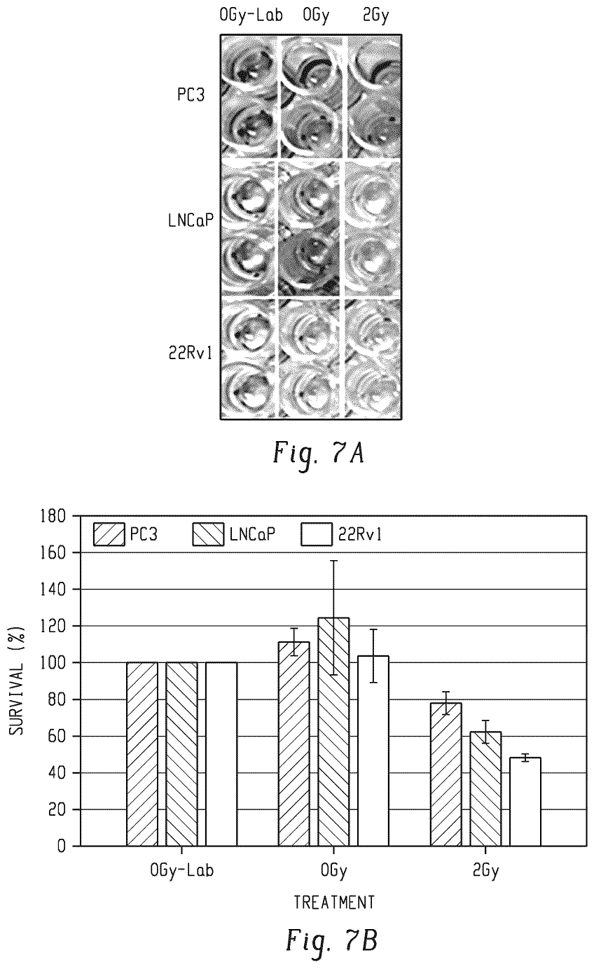

The identity of exosomes isolated by exoEasy was confirmed by analyzing the expression of an exosome marker, CD81 (one of the tetraspanins) from the protein lysate of isolated exosomes (FIG. 2). The abundance of exosomes isolated from three cells using exoEasy and ExoPur™ was determined by quantifying the exosomes shown in FIG. 3 and FIG. 4 respectively. The results obtained with ExoPur™ were comparable with Qiagen's exoEasy.

In order to demonstrate that exosomes from each cell line represent the inherent characteristics of the cell lines, the identity of isolated exosomes by western blots was confirmed. The protein lysate of isolated exosomes was analyzed for the expression of an exosome marker, CD81 (one of the tetraspanins) (FIG. 1A). Since these 3 PCa cell lines are known to have differential basal PD-L1 expression, the expression of PD-L1 from CCM derived exosomes for all three prostate cancer cell lines was analyzed using western blot (FIGS. 1B & C) and demonstrated that exosomes derived from CCM of 3 PCa cell lines represent the inherent quality of the cell lines.

### Example 2: Confirming that the 3 PCA Cell Lines Show Differential RS to Radiation

The selected 3 PCa cell lines have been shown in literature to differ in their radiation sensitivity (RS) at 2Gy. In order to confirm that all 3 cell lines show sensitivity to the same radiation dose in the radiation set up, and in order to classify the cell lines accurately based on their radio-sensitivity and to set the baseline for the biomarkers for distinguishing the changes resulting from radiation, (instead of stress induced changes due to transportation of flasks), an additional un-irradiated control was implemented.

Cells pooled from multiple flasks were seeded in 6 well and 96 well plates to conduct the clonogenic and proliferation assays, respectively. One full plate with all 6 wells was used for one cell line per treatment, giving 6 independent reads. We first examined the difference in radio-sensitivities of the 3 cell lines by their ability to form colonies using the clonogenic assay, the gold standard for determining radiosensitivity. Differences in colony formation capacity of three PCa cell lines upon irradiation to 2 Gy are shown in FIGS. 2A & B (p-value <0.01 using two factor ANOVA). The percentage colony formation was calculated from the proportion of colonies present after treatment in comparison to colony numbers within the un-irradiated control sample kept in the lab (0 Gy-Lab). No substantial change in colony formation capacity was observed for PC3, while LNCaP and 22Rv1 showed 46% and 70% reduction in colony formation after exposure to 2Gy radiation. Based on the colony formation ability of PCa cells to 2Gy, the PCa lines into were categorized into three categories: no change/less than 20% reduction in colony formation as low radio-sensitive/radio-resistance (PC3), ≤50% reduction in colony formation as medium radio-sensitive (LNCaP) and >50% reduction as highly radio-sensitive (22Rv1). A total of 4 independent experiments were performed at different times.

Next, cell proliferation assays were used (WST-8 assay kit, Dojindo Molecular Technologies, Inc., MD) to evaluate the effect of radiation on changes in the status of live and viable cells. We calculated the survival % using: ([As−Ab]/[Ac−Ab])×100 (As: sample absorbance; Ab: Absorbance blank; Ac: Absorbance negative control). The differential sensitivity of cell lines to 2 Gy radiation (p-value <0.01) is shown in FIGS. 3A & B. Based on survival % following 2 Gy irradiation, PC3 showed the greatest level of resistance to radiotherapy (≥75% cell survival), LNCaP showed medium sensitivity (>50% and less than 75% cell survival) and 22Rv1 showed high sensitivity (less than 50% cell survival). For the cell proliferation assay, different cell concentrations for each cell line were tested in triplicates, for each treatment. Three independent experiments were performed at different times.

Based on radio-sensitivity determined by using clonogenic and proliferation assays, the 3 PCa cell lines were classified into 3 different categories (PC3, low radio-sensitive/radioresistant; LNCaP, medium radiosensitive, 22Rv1, highly radio-sensitive).

### Example 3: Establish Biomarker Profiles for all 3 Cell Lines to Radiation at 2Gy

Based on the standardization on quantification of the exosomes from CCM, different amounts of exosomes from all 3 cell lines were tested to establish the amount of exosomes required to quantify the proposed biomarker panel by real time PCR. We performed a series of radiation experiments to isolate sufficient amounts of exosomes from irradiated cell lines. We found that 35-40×10⁸ exosomes are sufficient to perform real time PCR quantification of the proposed biomarker panel from CCM. We standardized the isolation of RNA, cDNA synthesis and the amount of cDNA template required to monitor changes in the biomarker panel from exosomes isolated from CCM for un-irradiated and irradiated samples.

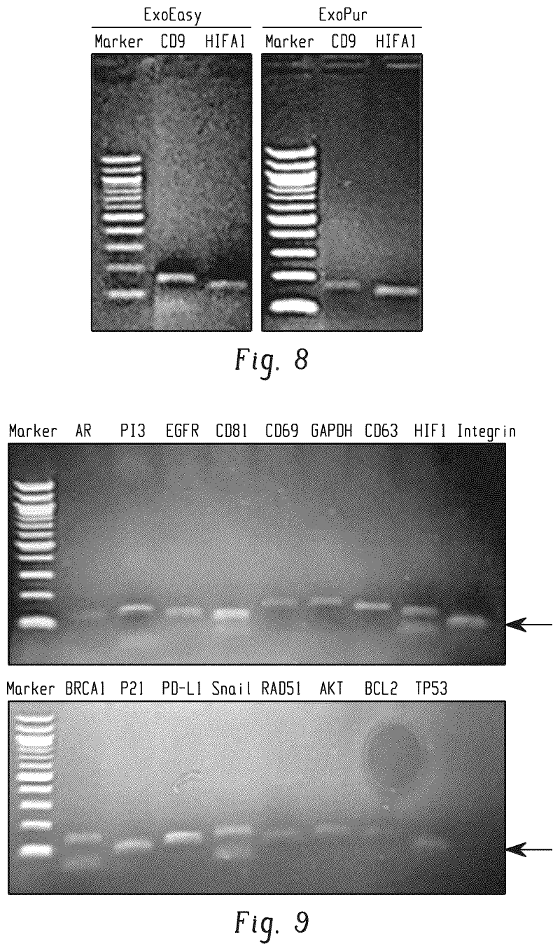

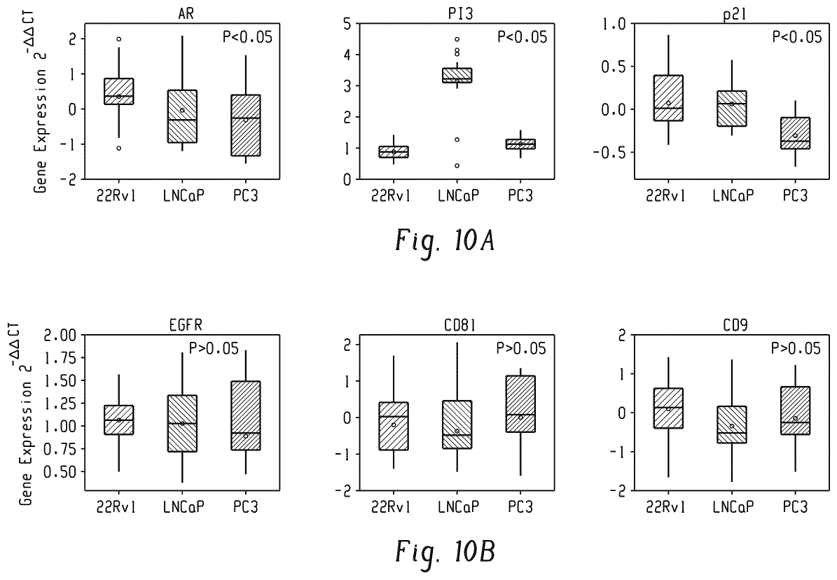

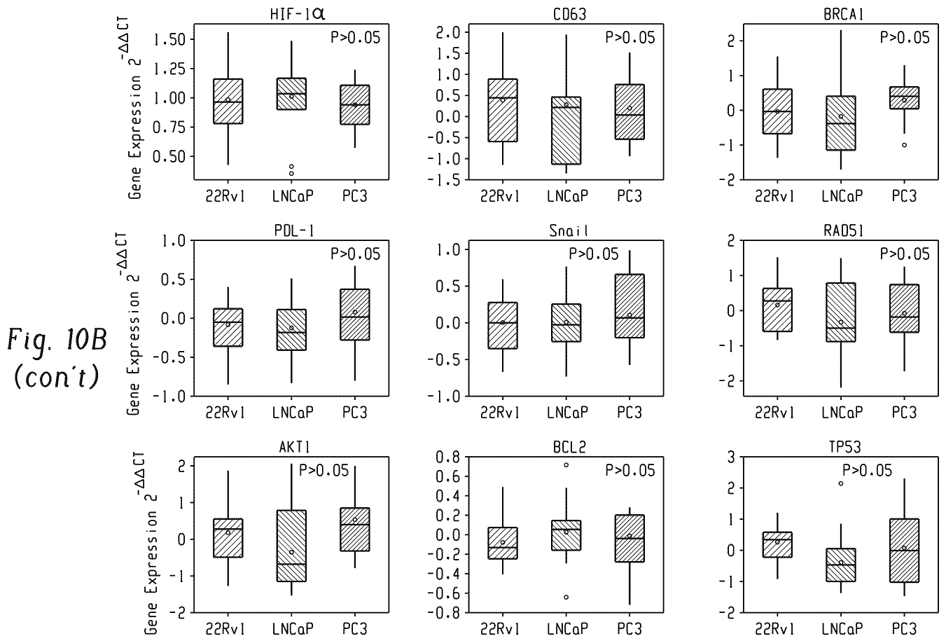

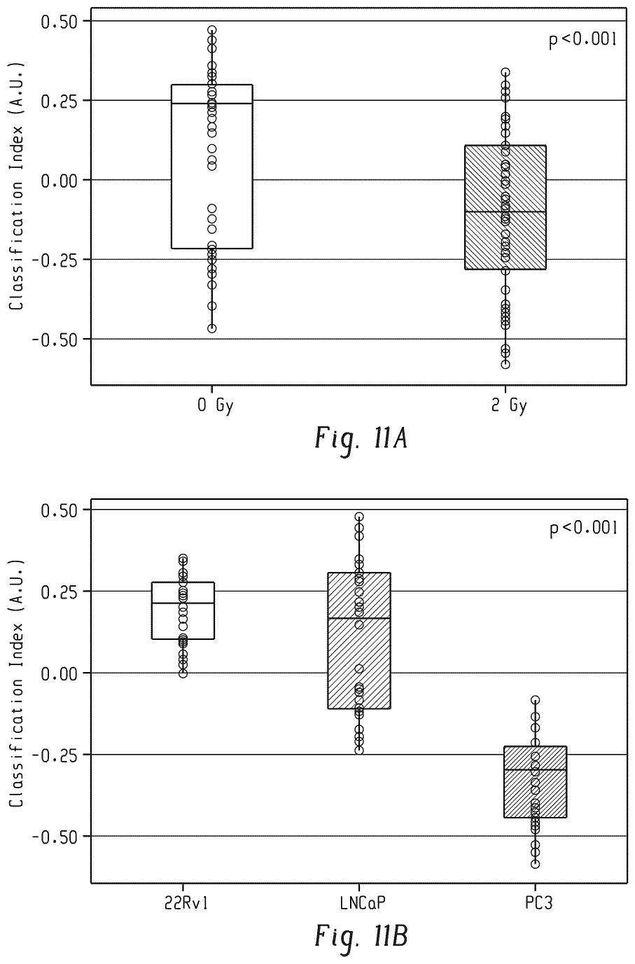

Using the Taqman master mix, we first tested the real time PCR (qPCR) reaction for biomarkers, from exosomes isolated using exoEasy and ExoPur (FIG. 8). Next, we confirmed the size and specificity of the qPCR product for each biomarker by running the PCR product on 1.5% gel (FIG. 9).

Based on the radio-sensitivity to radiation at 2Gy, using clonogenic and cell proliferation assays, the three cell lines were successfully classified into: low sensitive (resistant) PC3, medium sensitive LNCaP and highly sensitive 22Rv1.

To clearly demonstrate the ability to monitor the changes in biomarker expression profile from exosomes, 3 additional exosomes markers (CD9, CD81 and CD63) were included in the biomarker panel.

Based on P-value, the relative changes in biomarker expression were categorized into 2 different groups: P value ≤0.05 and P value >0.05 (FIG. 4A-B).

Data Analysis of Cell Responses to 2Gy using MANOVA: A single biomarker is unable to reflect the heterogeneous nature of PCa and response to radiation. In order to capture the multi-dimensional view of radiation response to three PCa cell lines showing variation in their radio-sensitivities, the changes in multiple biomarkers were monitored simultaneously, and analyzed the measurements using a two-way multivariate analysis of variance (two-way MANOVA).

A two-way MANOVA analysis (independent variables: dose {0, 2Gy} and cell lines {PC3, LNCaP and 22Rv1}; responses: signals from 16 biomarkers) was used to determine if the biomarker patterns corresponding to the 3 cell lines were distinguishable from each other. A total 8 independent experiments, 17 samples were used per radiation dose for PC3 and LNCaP, while the sample number for 22Rv1 was 18 per radiation dose. Prior to analysis, the data were examined for outliers, and subject to normality tests such as Lilliefors. For data violating the p>0.05 criterion, a Box Cox or Johnson transform was used. Multivariate normality was assessed using Henze-Zirkler's test, and Levene tests were done to verify variance homogeneity, followed by multi-collinearity tests. The output of MANOVA using Minitab showed p<0.001 for all 3 test criteria—Wilks', Lawley Hotelling and Pillai's test statistics values were 0.01071, 29.93864, 1.65780 respectively. Eigen Analysis was used to select the biomarkers that contributed to 90% of the differentiation.

MANOVA analysis using delta CT values for all the biomarkers (AR, PI3, EGFR, CD81, CD9, HIF-1α, CD63, Integrin, BRCA1, p21, PDL-1, Snail, RAD51, AKT1, BCL2, TP53) indicated that the biomarker panel (PI3, CD81, CD9, HIF-1α, CD63, BRCA1, p21, PD-L1, AKT1 and BCL2) in combination elicits a response to radiation (FIG. 11) and allows categorization of the three cell lines into three different groups (FIG. 11). Post hoc testing of the (LCM) linear combination model of this biomarker panel with the Dunnett method (due to inherent variance homogeneity) showed that 0 Gy and 2 Gy groups are significantly different from each other at a CI (confidence interval) of 95%. The Games-Howell test (used for unequal variance between cell line groups), at 95% CI reveals that all three cell lines belong to three different groups which are significantly different from each other. The results obtained herein are in agreement with classification of the cell lines into three different categories: low sensitive (resistance): PC3; medium sensitive: LNCaP; and highly sensitive: 22Rv1, at 2Gy.

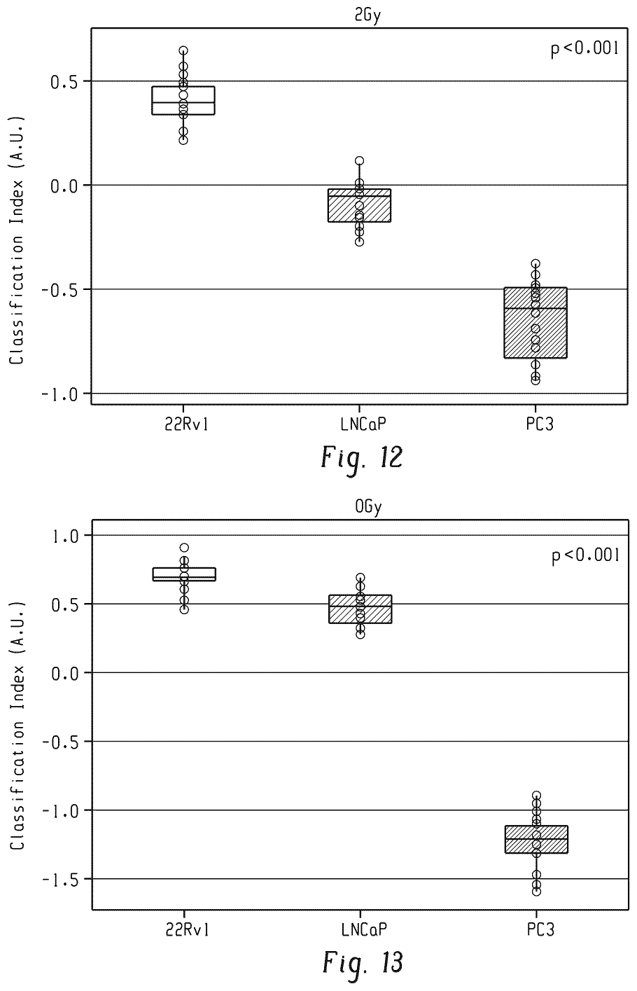

Based on delta CT, MANOVA analysis, the biomarker panel (AR, PI3, EGFR, HIFA1, CD63, p21, PDL-1, RAD51, AKT1 and BCL2) in combination, categorized the cell lines into three different groups at 2 Gy (FIG. 12). Post hoc analysis of the LCM with the Games-Howell test at 95% CI confirms that all three cell lines belong to three different groups which are significantly different from each other. The results obtained herein are also in agreement with classifying the cells into 3 different categories: low sensitive (resistance), PC3; medium sensitive, LNCaP; and highly sensitive, 22Rv1, to 2 Gy.

MANOVA analysis using delta CT values shows that the biomarker panel (AR, PI3, EGFR, CD9, CD63, p21, PDL-1, Snail, RAD51 and TP53) in combination, can mimic the categorization of cell lines into three groups, similar to 2 Gy (FIG. 13). The Dunnett test at 95% CI further confirms the results of separating the three cell lines into three different groups, which are significantly different from each other at 0 Gy.

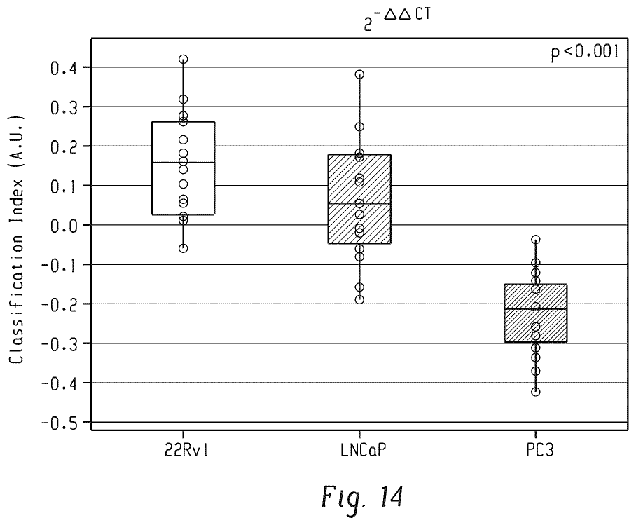

MANOVA analysis using a relative quantification strategy by employing delta delta CT method (2^{-ΔΔCT}) reveal that the biomarker panel (AR, EGFR, CD81, BRCA1, p21, PDL-1, Snail, RAD51, AKT1 and TP53) categorized the cell lines into two groups (FIG. 14). The Dunnett test at 95% CI (using 22Rv1 as the control group) further corroborates that the three cell lines fall into two groups, which are significantly different from each other. The results obtained herein indicate that the PC3 cell line was classified into resistance and LNCaP and 22Rv1 were classified as sensitive (LNCaP as medium sensitive and 22Rv1 as highly sensitive).

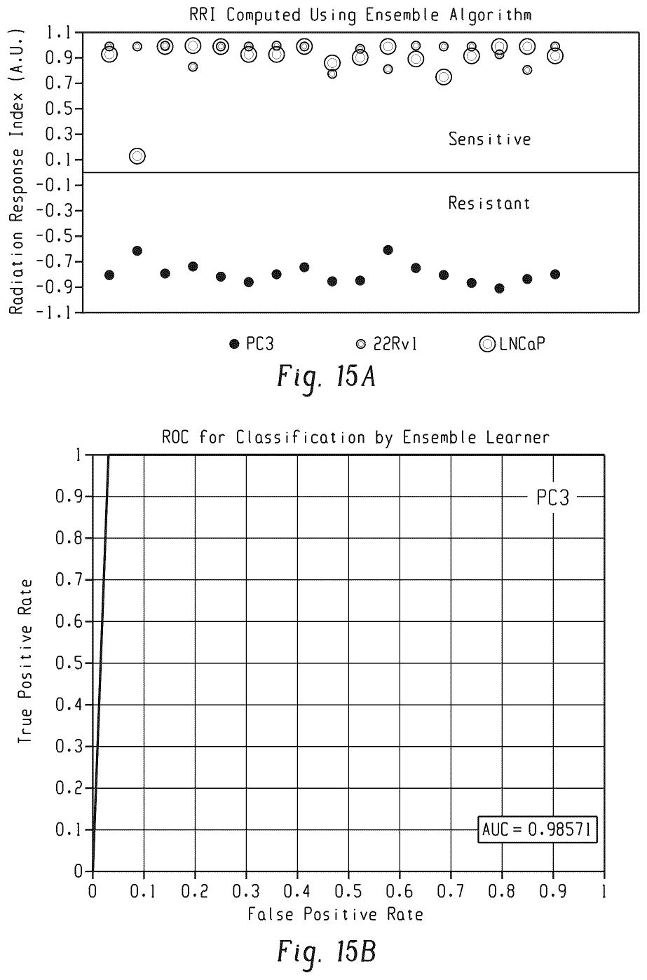

Data Analysis to Calculate Radiation Response Index (RRI): Next, the delta-delta CT values of biomarkers segregated through Eigen Analysis was used as inputs into various machine learning techniques for discriminant analysis, such as k-nearest neighbors, Naive Bayes, Support Vector Machine, and Ensemble Learners (result of boosting 100 classification trees) using scripts in Matlab, for a finer distinction. We used CV (Cross Validation) Errors and AUC (Area Under the Curve) for ROC (Receiver Operating Curve) as metrics to assess the performance of each model developed, and the best was adopted to generate an RRI (Radiation Response Index). K-fold partitioning of the data was done during the model development, in order to avoid overfitting. Thus, machine learning used the relative changes in the expression (2^{-ΔΔCT}) of the cell lines as predictors and radiation resistance (RR)/radiation sensitivity (RS) as the response. The biomarker panel based RRI correctly classified all the PC3 samples as resistance and 22Rv1 and LNCaP as sensitive (FIG. 15). An example ROC and AUC for PC3 is shown in FIG. 15), providing a value of 0.99. Hence, the biomarker panel (AR, EGFR, CD81, BRCA1, p21, PDL-1, Snail, RAD51, AKT1 and TP53) was identified which categorizes/differentiates the cell lines based on their sensitivity to radiation.

The importance of each predictor is shown in FIG. 1 when a combination of 16 biomarkers are used; ‘2’(PI3) and ‘10’(P21) standout, followed by 16(TP53),9(BRCA1),14(AKT),4(CD81), 1(AR). While the combination of 10 biomarkers (AR,EGFR,CD81,BRCA1,P21,PD-L1,Snail,RAD51, AKT, TP53) with ΔΔCT values resulted in a very low error rate (2 non-critical misclassifications of the high and medium sensitive samples, one misclassification of a medium sensitive sample as resistant, and no misclassification of any resistant samples) using the Ensemble Learner algorithm, a combination of the remaining primers within the panel (PI3,CD9,HIF-1α,CD63,Integrin,BCL2) did reasonably well, albeit with a higher error rate, with a critical misclassification of resistant as sensitive, 2 misclassifications of sensitive as resistant, and one non-critical misclassification of medium sensitive as sensitive. The combination selected for use mentioned above predicted all the radiation resistant samples correctly, which is a critical factor. Reducing the number of biomarkers generally results in a reduction in the AUC, and in the classification margins (separation between classes), which means lower robustness.

Similar to the above employed Ensemble Learner based model, a linear combination algorithm also resulted in a trend of inferior models developed using individual biomarkers when compared to a model using all of the above biomarkers. Other models may be developed (based on machine learning algorithms such as support vector machine, naive bayes, neural network, etc. as known to those versed in the art, or discriminant analysis algorithms based on classical statistics).

The correct categorization of cell lines based on their radiation sensitivity successfully demonstrate the potential of the biomarker based RRI to stratify the patients based on individual radio-sensitivities, to fulfill a current unmet need of radio-oncology.

### Example 4: Identify Changes in Biomarkers Profile to Radiation at Fractionated Dose of 2Gy Once a Day, for 5 Days, in 3 PCA Cell Lines

Radiation Therapy (RT) is the most employed treatment modality for prostate cancer and is typically administered in daily fractions for approximately 8 weeks (5 days/per week). Most men get a minimum total dose of 75.6 Gy, which translates to 2 Gy or less per day. In order to mimic a clinical setting, a fractionated radiation protocol (delivering 2 Gy on successive days for 5 days) was used to increase the dose slowly and monitored the changes in biomarker profiles of 3 PCa cell lines after each irradiation leading to the identification of specific biomarker signatures associated with different RT doses.

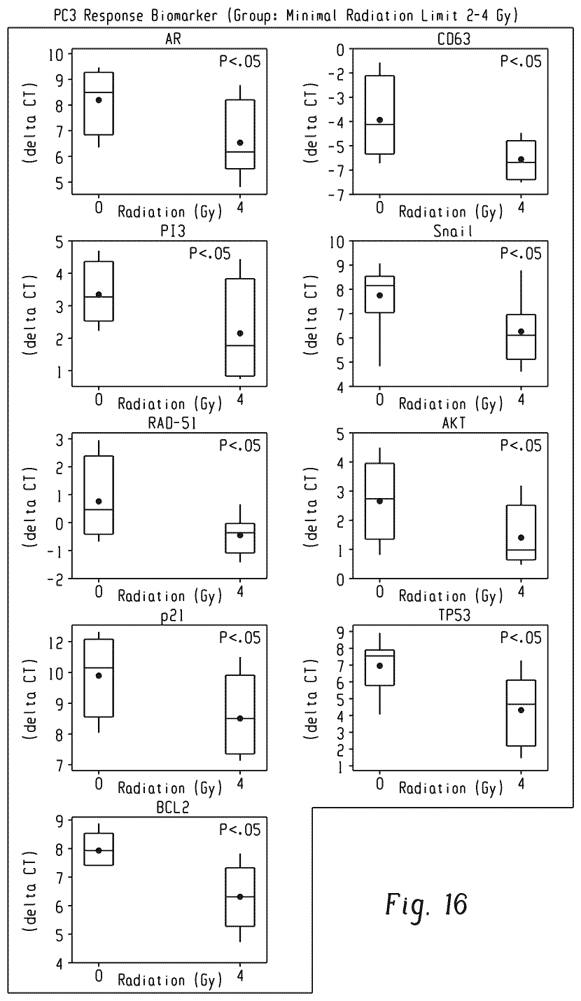

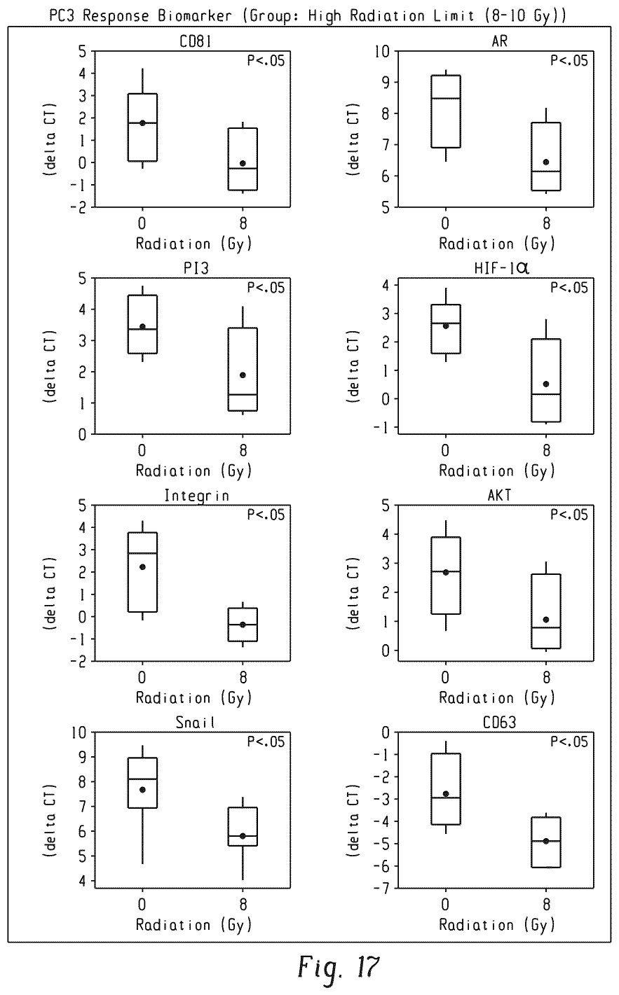

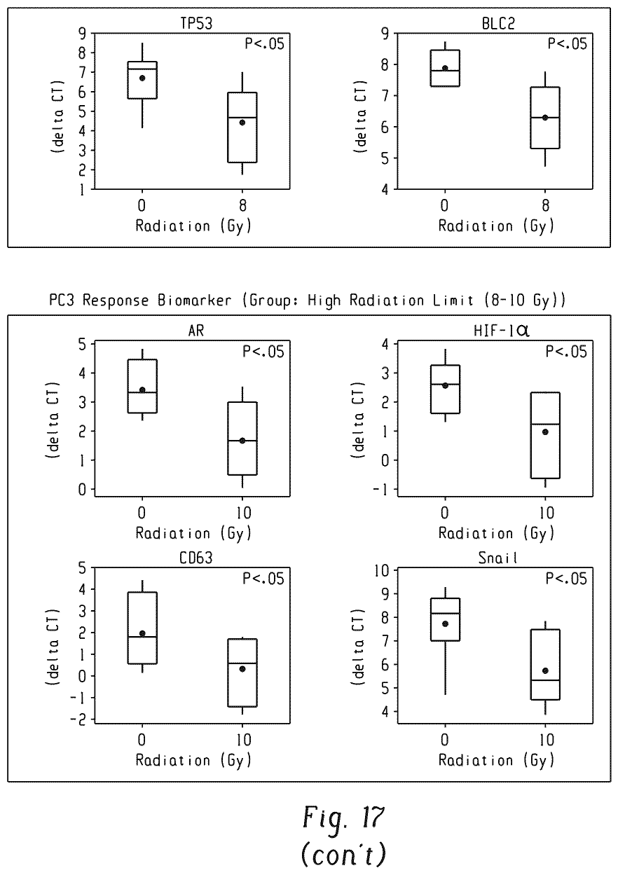

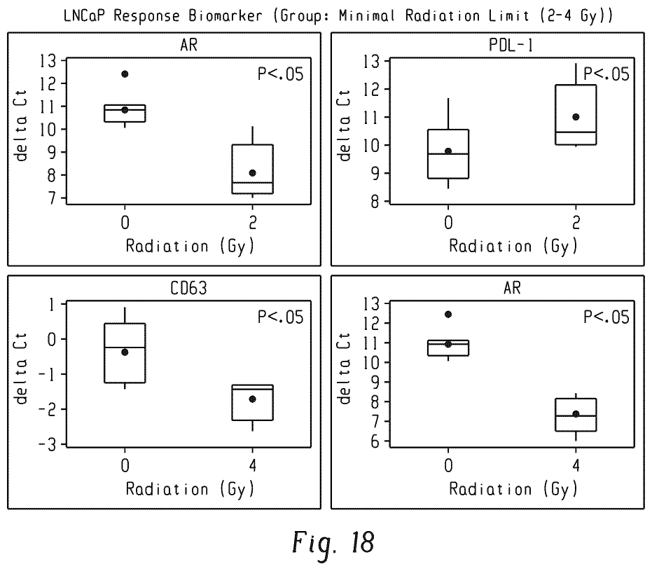

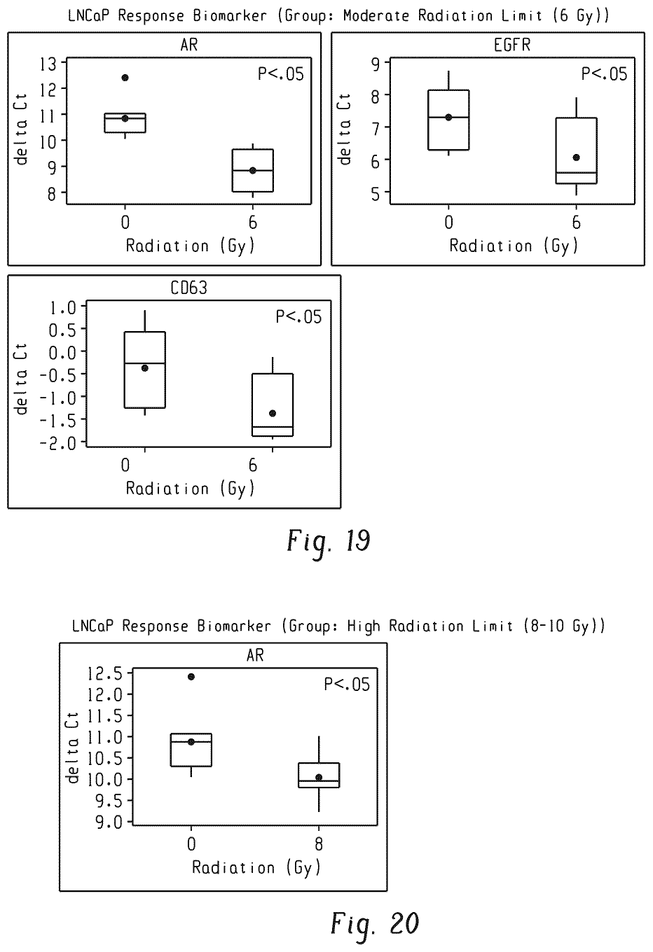

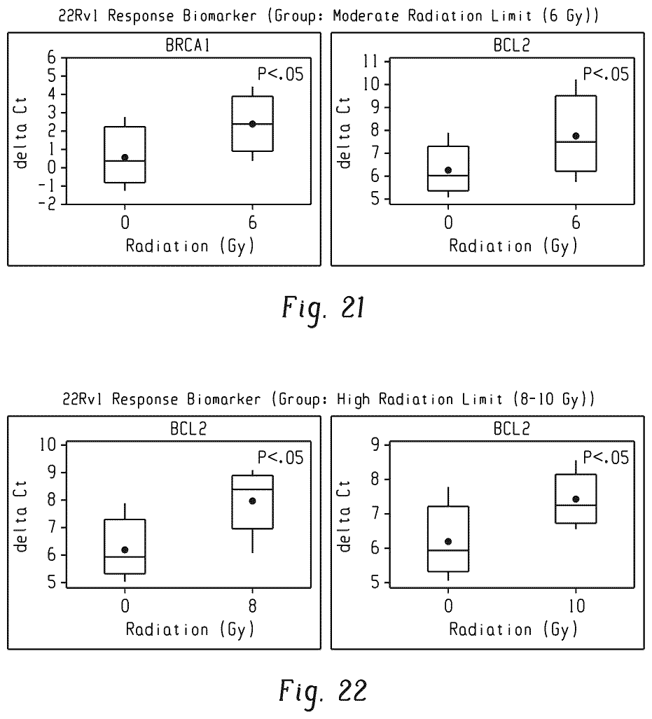

To mimic a clinical setting where the radiation dose of 2 Gy is delivered on successive days, a fractionated radiation protocol was used to increase the dose slowly and monitor the changes in biomarker profiles of 3 PCa cell lines after each irradiation. The 3 prostate cancer cell lines were irradiated at 2Gy/min once a day for 5 days. Cells for each cell lines were seeded as per methods described above. The flasks of each cell line were set up in triplicates at the start of the experiment and cells were exposed to 2 Gy radiation dose for 5 consecutive days. No radiation was given to control flasks. After irradiation of 2 Gy each day, the flasks were transferred to 37° C. incubator, 5% CO2, and conditioned media with exosomes was collected after 48 hours. A minimum of 3 flasks for each cell line were used to collect the condition media. The isolation of exosomes was performed and changes in expression of biomarkers by qPCR was performed as described above. To establish radiation specific biomarkers, radiation levels were classified into minimal radiation level (2-4 Gy), moderate radiation level (6 Gy) and high radiation level (8-10 Gy). FIGS. 16-17 show the established dose specific biomarker panels for the radiation resistant (PC3) cell line. FIGS. 18-20 show the established dose specific biomarker panels for radiation sensitive (medium) cell line. Figures. 21-22 show the established dose specific biomarker panels for radiation sensitive high (22Rv1) cell line. Eight independent observations from each cell line per dose were used to perform the analysis.

### Analysis to Identify Different Dose Specific Rt Response Signatures

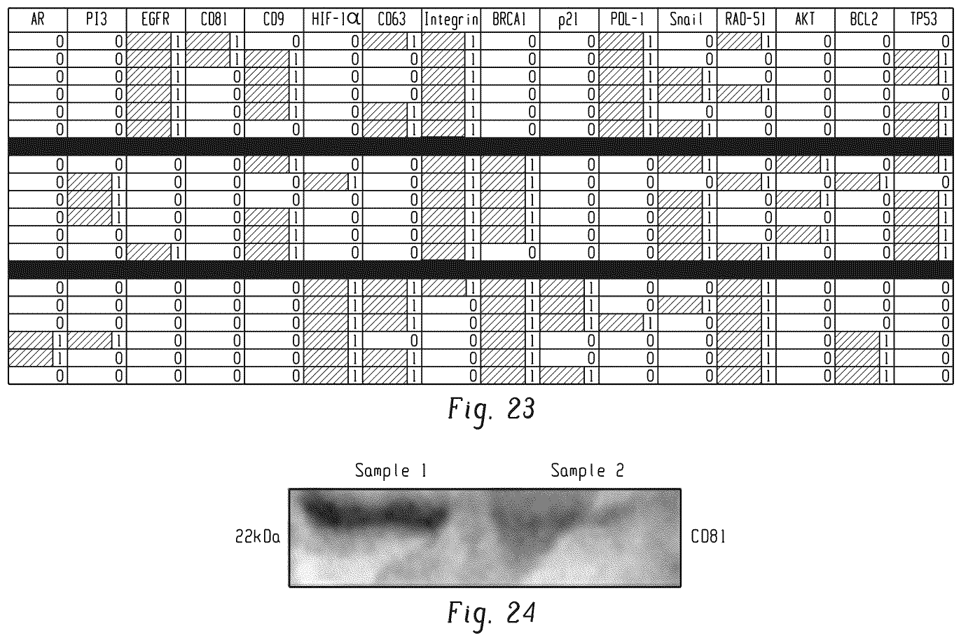

The 8 independent observations per Radiation level/dose, and all 14,892 combinations of 1 to 6 predictors (from a total of 16) were iteratively run to determine the maximum pairwise distance between each Radiation level/dose group and the non-radiated group, using a Matlab script. The predictor combination corresponding to the maximum distance was extracted for each Radiation level/dose for each cell line. A heat map of predictors obtained for each cell line at each specific dose enabled visualization of the difference in biomarker profiles (FIG. 23). The turning ON and OFF of different biomarkers and the constancy of involvement of certain others with increasing radiation dose is evident. The biomarker profile of the medium radiation sensitive cell line was noted as a mix of the biomarker profiles of the radiation resistant and highly radiation sensitive cell lines.

We next performed the inter and intra assay precision for biomarkers and the precision of the method based on intra and inter-assay was 2-6% and 1-15%, respectively.

### Example 5: Testing and Validating the RS Prediction in Clinical Samples

Sample size: For a power of 0.95, using the current cell line data as an exemplary dataset, the calculated total sample size was 48. The sample size calculation for two-way MANOVA using delta CT was performed using the tools from real-statistics.com. For MANOVA analysis, the two independent variables (cell lines and radiation) represent a total of 4 groups (cell lines (resistant & sensitive); radiation (0 Gy & 2 Gy). The assumption, based on the 2Gy results, was that 10 predictors would be enough to develop the Radiation Resistance Index (RRI); hence a minimum of 12 samples per group for a total of 4 groups was estimated.

The sample size calculation for the discriminant (multivariate) analysis, which was based on relative changes in biomarker expression (2^{-ΔΔCT}), worked out to a total of 80. The proposed selection of a sample size of 80 provided us the flexibility of using both methods, satisfying the criteria for analysis of each method. Though a sample size of 80 was proposed, the experiments were done on a total of 81 samples. Detailed information on all 81 samples, comparing the clinical status with RRI performance was obtained.

Sample details: Bio specimens utilized were clinical surgical excess or remnant human bio specimens that are obtained during the course of standard medical care or follow-up. Based on the source of samples, names & numbers were assigned to the samples. The clinical sample pool consisted of samples collected before RT {treatment naive, denoted with the suffix ‘A’ in the sample name}, after radiotherapy/irradiation, and during RT (denoted with suffixes B-D at the in the sample names, based on the point of collection).

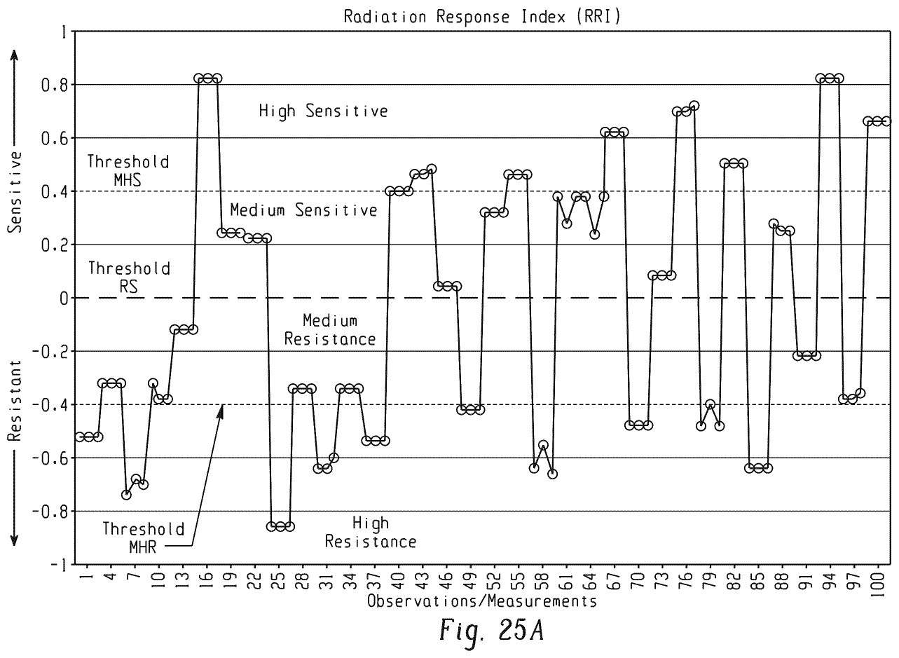

Initial standardization of human samples for isolation of exosomes: We first established the identity of isolated exosomes by western blots and qPCR from human serum sample by analysis of expression of an exosome marker. A western blot analysis with exosome marker CD81 is shown in FIG. 24.

Collection and testing of human samples to validate the utility of RRI based categorization in clinical samples: The purity of nucleic acids extracted from individual samples can strongly affect correctness and repeatability of RT-qPCR quantification. To prevent the clinical data from being biased by inhibition, G-block of Luciferase (gene absent in human) were synthesized and used in vitro transcribed mRNA as an RNA spike. This approach allowed us (a) to verify the parameters of the workflow each time; (b) validate the purity of RNA after extraction, and (c) detect the presence of any inhibitors.

The RRI index method developed to categorize the clinical samples into radiation resistant and sensitive categories. However, employing a delta CT based method was more appropriate for this task. Using this approach, human samples were handled where a comparable pre-treatment sample for each radiation treated sample was unavailable.

Different methods were first assayed to develop a cogent comprehensive model with all 6 radiation doses and 3 cell lines and the following approach was selected: all the radiation levels (2Gy-10Gy) were grouped together and used all the 40 observations available per cell line to develop a group of Radiation-based models to differentiate between radiation resistant (PC3) and radiation sensitive (22Rv1) categories. For the non-radiated group, the 17 observations in PC3 and 18 observations in 22Rv1 were used to develop a separate group of Non-Radiated models that distinguished between PC3 and 22Rv1 as described above. Using this two-group approach, the models were applied separately to the human samples, which were also known apriori to be either Pre-Treatment, or Post Treatment. Extensive work was done to reduce these down to two best suited models applicable to the human data, based on the limited available “Ground Truth” comprised of clinically relapsed cases, and some clinically stable cases that had the benefit of longitudinal data collection, to logically deduce the state of certain pretreatment samples.

### Example 6: RRI Test Prediction Before Treatment

Based on clinical information, the treatment naive samples were separated as a pretreatment group and analyzed for prediction. The pre-treatment model using a biomarker panel consisting of 4 (AR, HIF-1α, CD63, p21) out of a total of 16 (listed in Table 1) was determined to be among the best to calculate the RRI for human samples.

Besides this combination, several other good combinations of biomarkers were obtained which categorized the radiation resistant and sensitive samples in a similar manner as mentioned above (Table 2). The initial sample size determination was done with the assumption of 10 predictors using the delta-delta CT method. However, the extensive analysis done utilizing the apriori knowledge of the clinical data of the human samples using delta CT reduced the biomarker combination to 4 predictors. The sample size, using the same rule for multivariate analysis employing machine learning algorithms, with 4 predictors and 2 groups is 4×4×2 or 32. In this study, 34 pre-treatment samples were analyzed with 3 independent reads.

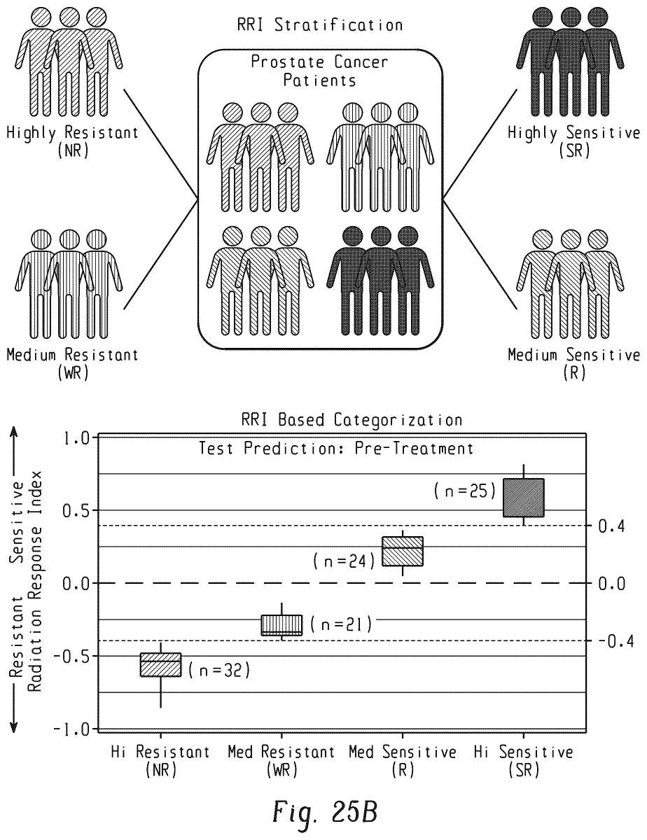

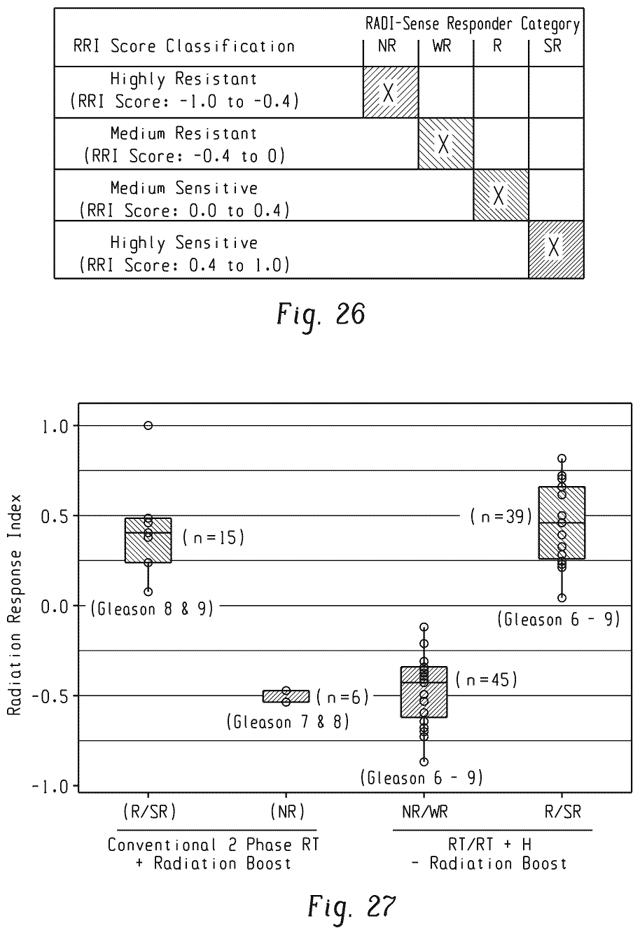

Based on clinical information, 34 treatment naïve samples (total 102 independent reads), (sample size power, 0.95) were successfully categorized into RR and RS categories (FIG. 25) using a pre-treatment RRI algorithm. The granularity in classification, in terms of RRI, based on the resistance/sensitivity profiles of all pre-treatment samples is illustrated in Figure FIG. 25B is a box plot of the same data, classified in different groups based on their sensitivity by RRI (Highly resistant as non-responder (NR); Medium resistant as weak responder (WR); Medium sensitive as responder (R) & highly sensitive as Strong responder (SR). An example of Response Prediction Matrix using the RRI for treatment adjustment based on radiation sensitivities is shown in FIG. 26. NR unlikely to respond RT; WR, likely to respond with dose escalation; R, responder likely to respond to standard dose; SR, strong responder likely to respond even with dose-de-escalation.

Treatment of intermediate and high-risk clinically localized PCa represents a clinical challenge. The conventional RT for these patients is carried out in a sequential two-phase process. Following a definitive therapy both intermediate- and high-risk patients show a relatively high rate of recurrence. Identification of a pre-existing, intrinsic radio-resistance in PCa will help to develop optimal treatment strategy to reduce the risk of local recurrence. To see if the pretreatment RRI based classification can guide the dose adjustment decisions the pre-treatment RRI predictions were used to sub-classify the patients based on additional radiation treatment (Boost). FIG. 27 shows the prediction from pre-treatment samples with and without Radiation Boost. The Gleason score is indicated. The RRI categorized both with and without Boost categories into RS and RR. Based on analysis of clinical information/data, RRI-based pre-treatment prediction for all the samples from Responder category (with Boost) showed positive outcome for patients treated with conventional 2 phase RT and predicted all the Responders for RT/RT+Hormone (H) treatment (without Boost category) correctly.

High risk PCa account 22.3% of the diagnosis but for 66.2% of PCa deaths within 10 years of diagnosis, the RRI-based prediction will help in making right boost decisions for patients who can get maximum benefit and reduce mortality.

According to the phase 3 FLAME study, addition of an extra dose (Boost) to the standard RT improves biochemical disease-free survival in PCa. Several studies have additionally demonstrated that dose escalation improves biochemical tumor control outcomes,

### Example 7: RRI-Based Prediction Post-Treatment

Based on clinical information, the samples obtained after radiation treatment were segregated as the posttreatment group and analyzed for RRI-based prediction. A post-treatment model using 4 biomarkers (AR, PI3, CD63, p21) out of a total of 16 (listed in Table 1) was developed and used to calculate the RRI. Besides this combination, several other good combinations of biomarkers were obtained which categorized the radiation resistant and sensitive samples in a similar fashion mentioned above (Table 3).

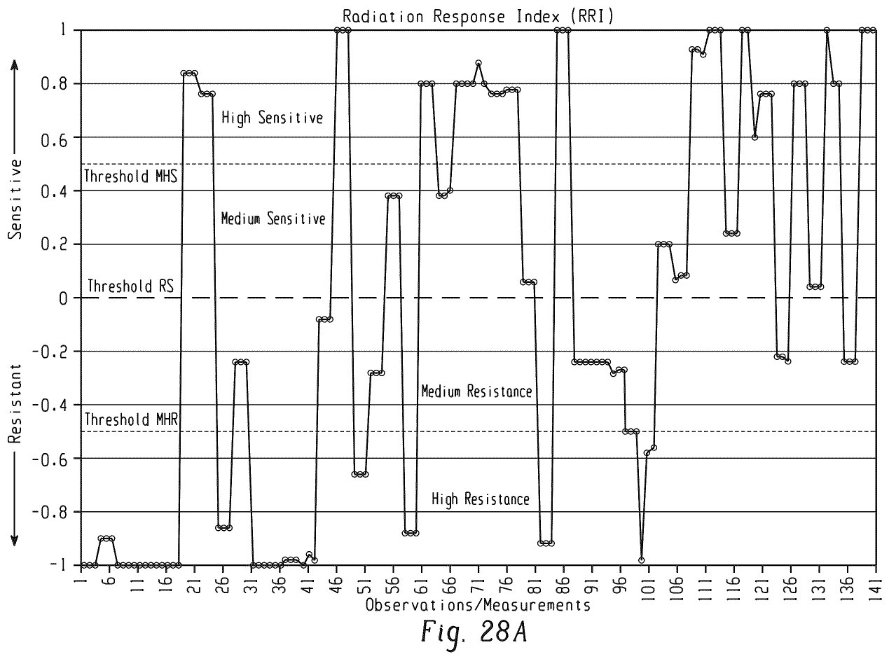

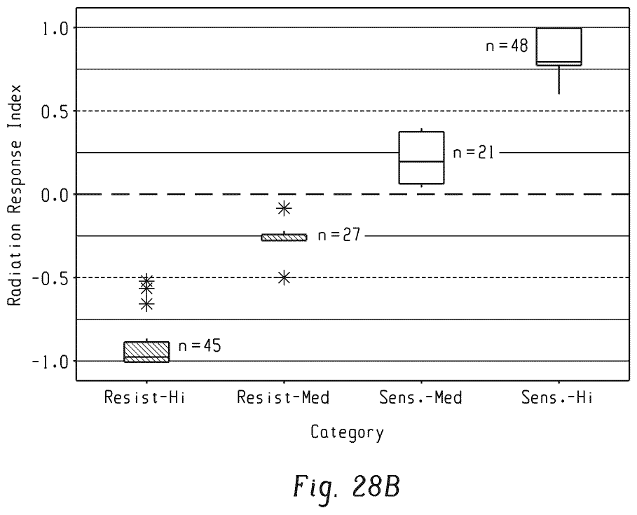

The sample size, using the same rule for multivariate analysis employing machine learning algorithms, with 4 predictors and 2 groups is 4×4×2 or 32. In this study, 47 post-treatment samples were analyzed with 3 independent reads—which is more than sufficient. AUC exceeding 0.95 and Cross-Validation errors <0.05 further confirmed that the sample size choice was adequate. The granularity in classification, in terms of RRI, based on the resistance/sensitivity profiles of all post-treatment samples is illustrated in FIG. 28A. FIG. 28B is a box plot of the same data, grouped in terms of High & Medium Sensitivities/Resistances.

### Example 8: RRI-Based Test for Treatment Monitoring Prediction

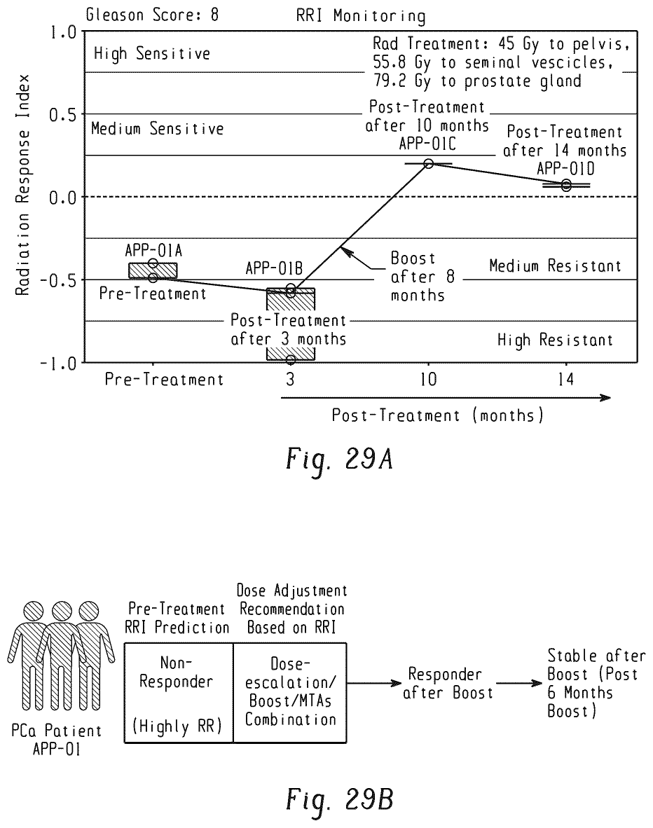

To demonstrate proof-of-concept on the functionality of RRI-based test for treatment monitoring prediction, the patient samples were first separated with longitudinal collection. Separate RRIs were obtained for treatment naive and post-treatment samples, using pre-treatment & post-treatment models respectively. The exosome-based biomarker approach allowed monitoring of a shift in molecular processes for the outcome of radiation treatment. The analysis of matching pre-treatment and post-treatment time intervals from the same patient (longitudinal sample collection) allowed evaluating the use of the test for monitoring the outcome of treatment. FIG. 29A shows a RRI treatment monitoring prediction for sample APP01. RRI classified APP01 sample as non-responder to RT before treatment and likelihood of responses to RT can be achieved by dose adjustment (dose escalation and/or MTAs combination therapy) FIG. 29B. The patient initially did not show response to therapy. Supplementation with Boost after 8 months resulted response to RT which the response to therapy. A response to Boost tested after 6 months shows stability in response.

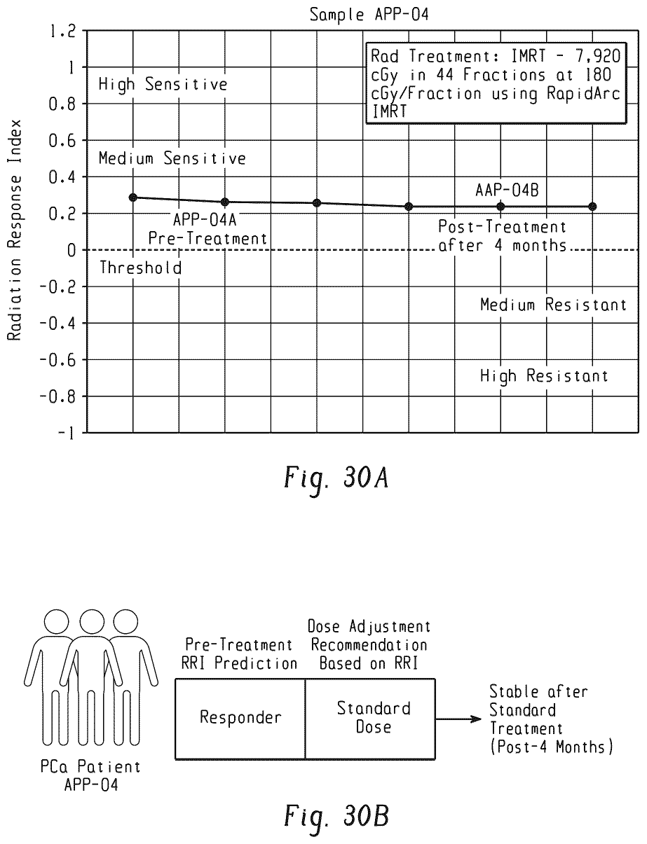

FIG. 30A shows a RRI treatment monitoring prediction for sample APP04. RRI classified APP04 sample as responder to RT before treatment and likelihood of responses to RT can be achieved by standard dose (FIG. 30B). The patient shows stable response to standard therapy (monitored after 4 months post-therapy). RRI monitoring and dose adjustment recommendations for non-responder and responder indicate the significance of our RRI-based prediction in assisting clinicians making treatment decisions.

The inter and intra assay precision for biomarkers in clinical samples was performed and the precision of the method based on intra and inter-assay was 2-6% and 1-13%, respectively.

This method was able to successfully classify 81 human samples into radiation resistant categories (high and medium) and radiation sensitive category (high and medium) based on RRI, with high precision (<15% CV for inter and intra assay).

### Example 9: RRI-Based Radiation Resistant Prediction for Early Relapse and its Comparison with Clinical Status

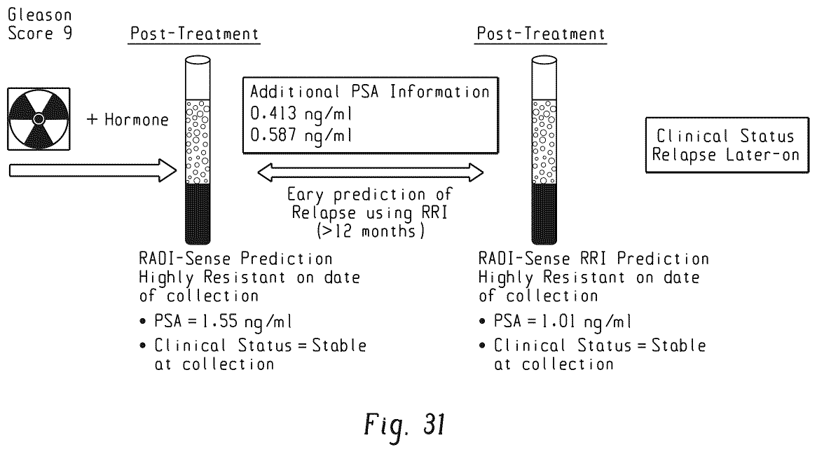

Data indicates that up to 50% of PCa patients undergoing radiation therapy experience recurrence of the disease within 5 years of treatment; hence the identification of early relapse is very important to provide alternative/combination options to patients and improve the treatment outcomes. By analyzing clinical samples collected from patients who were classified as relapse, the RRI-based test was able to predict the relapse at an earlier point of sample collection based on the post-treatment model (FIG. 31). The RRI-based test was also able to identify relapse even in cases where the PSA level was unable to provide accurate information (RT+Hormone).

### Example 10: RRI Prediction for Relapse Outperforms PSA (Current Gold Standard)

Treatment for locally advanced or high-risk prostate cancer results in less-than-optimal rates of disease control. Recurrence of high-grade prostate cancer after radiotherapy is a significant clinical problem, resulting in increased morbidity and reduced patient survival. An early relapse identification will allow the treatment initiation at molecular relapse without waiting for symptomatic incurable metastatic disease to develop. Serial measurements of prostate-specific antigen (PSA) and PSA-relapse/failure/biochemical failure has been a gold standard to detect disease recurrence patients who received RT. “Phoenix” criteria for PSA failure/biochemical failure is defined as a PSA rise by 2 ng/mL or more above the nadir PSA. The mean time for the PSA to reach its nadir is 18 months or longer.

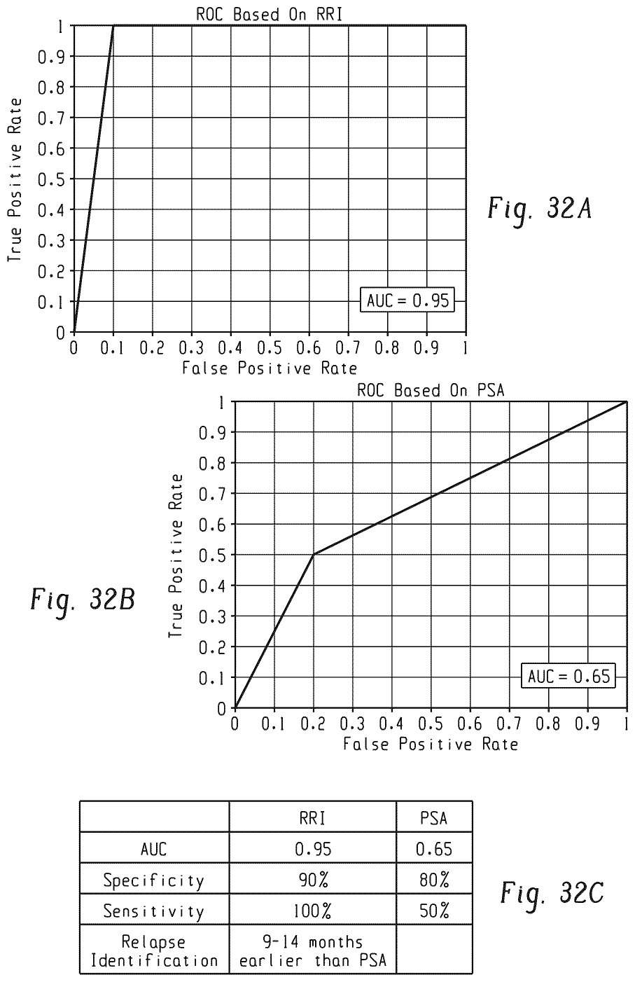

Using the post-treatment RRI algorithm, 47 samples were separated (141 independent read), (sample size power, 0.95) into RR (potential relapse) and RS (stable) categories. Assessment of radiation resistance after treatment will allow clinicians to be vigilant regarding change in patient's treatment options on time. Comparison of RRI-based test with the available clinical status of samples (stable/relapse) revealed that RRI-based test was able to predict all relapse cases correctly at an earlier point of sample collection (9-14 months earlier time) than the current gold standard PSA. The RRI-based test was also able to identify relapses correctly even in cases where the PSA level was unable to provide accurate information (RT+Hormone (H)/RT+Androgen Derivation Therapy (ADT)) but they were concluded as relapse at a later date. Comparison of the area under an ROC curve (AUC) of clinical samples with RRI to the use of PSA (current gold standard) showed that the RRI-based test shows the ability to predict recurrence with 100% sensitivity and 95% specificity (AUC=0.95, CI 0.87-0.99) and outperforms PSA (50% sensitivity; 80% specificity, (AUC=0.65, CI 0.51-0.78) (FIG. 32). Additionally, the RRI-based test was able to predict the relapse 9-14 months earlier in comparison to gold standard/clinical relapse. The better performance of the RRI-based test over the current gold standard and early relapse prediction combined with its ability to predict relapses correctly in case of RT+H/RT+ADT cases emphasizes the value of the RRI test in fulfilling the unmet need.

### Example 11: RRI Prediction and Biomarker Expression Guiding Combination Therapy to Provide Optimal Benefit

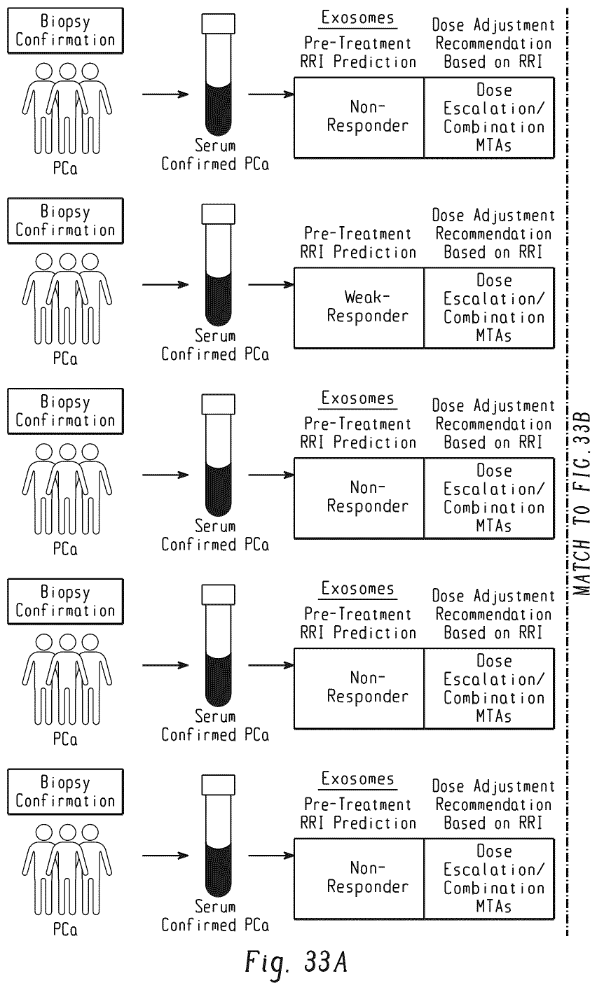

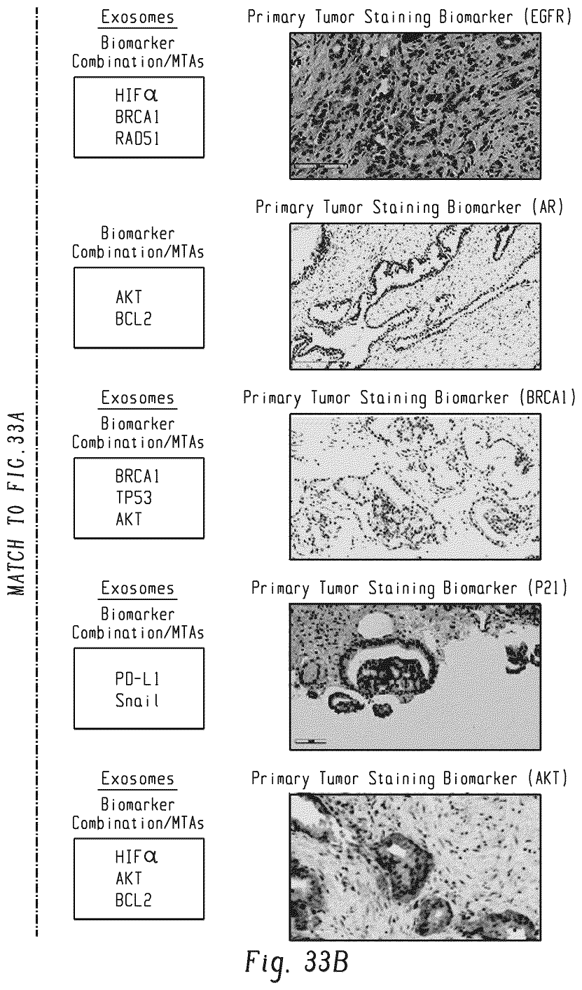

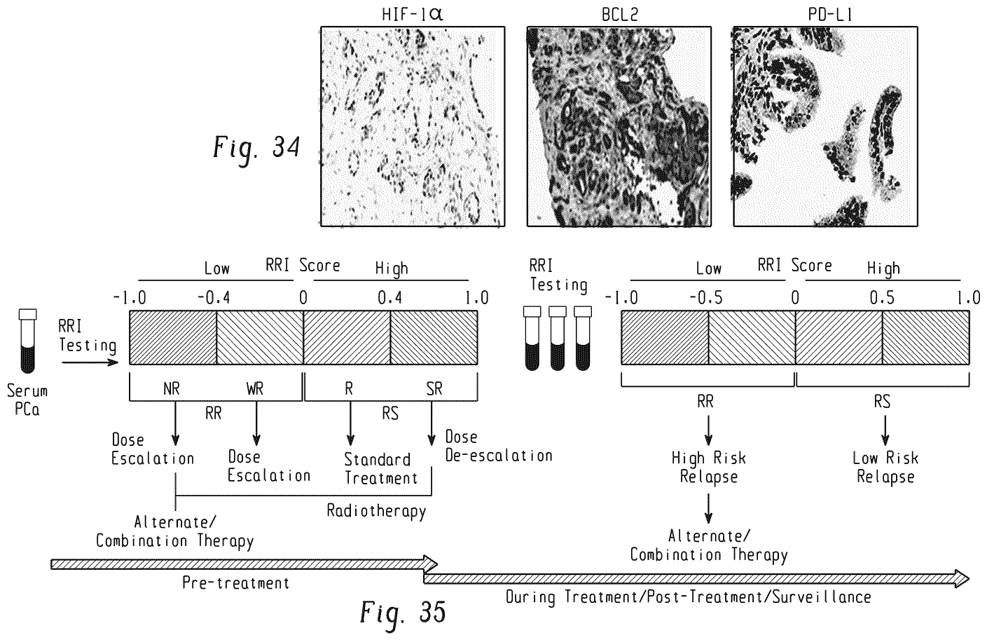

The subject/patients who do not respond to treatment and/or show failure to therapy after some time pose big hurdle for clinicians in making decision regarding their treatment plan. The use the biomarkers identifying specific molecularly targeted agents (MTAs) on these patients which can provide optimal benefit to non-responder and/or/weak responders and/or patients who stop responding to treatment and develop relapse will get benefit from individualized new therapeutic regimen. The biomarker based matched therapy option by analyzing the expression of biomarker on tumor cell provides the most effective treatment to patients based on the characteristic of their individual tumor ensuring the delivery of the right treatment to the right patient at the right time. FIG. 33 shows several samples which were categorized as either non-responder or weak responder by determining RRI using exosomes from liquid biopsies (serum/plasma) before RT. Use of exosomes-based gene expression for biomarkers from Table-1 as well primary tumor analysis by IHC allowed the identification of MTAs which can provide optimal benefit in these subjects. FIG. 34 show additional biomarker detection by IHC to guide combination treatment decisions.

The patients who respond to therapy initially develop also develop resistance (acquired radio-resistance) is a process of adaptation of cancer cells to the changes induced by irradiation itself, which finally results in resistance to the treatment. Identification of acquired resistance leading to relapse is a major challenge in getting the benefit from therapy. The RRI based monitoring and expression of biomarker analysis post-treatment offers a way to identify which patients are most likely to benefit from which combination therapy. Table-6 shows the identification of biomarkers from exosome using gene expression after post-treatment for determination of combination/MTAs therapies options for non-responder and weak responder. Predictive biomarkers which can correctly identify patients which are most likely to experience a favorable or unfavorable response from combination therapies are required for the success of personalized/precision therapy to overcome the risks of ineffective treatments which expose patients to undesirable side effects of treatments.

TABLE 6
EXOSOME BASED RRI POST-TREATMENT
MTAS FOR NON-RESPONDERS
Exosome based RRI Post-Treatment MTAs for Non-Responders

| Non-Responders Based on RRI | |
|---|---|
| Post-Treatment | Exosome Markers MTAs |
| APP-1 | HIF1-α, BCL2 |
| AMP-5 | AKT, BCL2, HIF1-α, RAD51 |
| AMP-9 | PI3, AKT, BRCA1 |
| ADPC-3 | PD-L1, TP53, BRCA1 |

The inventors have demonstrated that exosomes maintain the inherit quality of PCa tumor cells and can be used as non-invasive approach for longitudinal sample collection from patients prior to, during and after radiation treatment to monitor a continual response to RT. Any shift in molecular processes due to RT is reflected in exosomes and its status is helpful in evaluating the effectiveness of the given dose in real. Additionally, the success of this exosomes-based test to monitor the outcome of treatment indicates the potential of radical transformation of the current clinical standard for assessing response to RT, Response Evaluation Criteria in Solid Tumors (RECIST), which is based on measuring changes in tumor size is often not measurable until months.

For clinicians to be able to deliver biologically adapted, personalized precision RT for PCa patients they must be able to stratify patients based on individual tumor radiosensitivity before commencing treatment and be able to monitor RT responses during/post treatment to identify early relapses. Oncologists are in dire need of a simple and accurate test to evaluate high-risk patients that do not fall into normal treatment guidelines (i.e. High Gleason score but low prostate-specific antigen (PSA) in the case of prostate cancer). The inventors have identified a key panel of genes that can be used to create a biomarker indicative of response to RT therapy as well as for monitoring the ongoing development of resistance, which created the foundation for RRI, to stratify the subjects into clinically relevant categories, RR and RS. The method uses target exosomes from human plasma/serum, thus creating a platform for a simple and less invasive approach to regular testing that is more clinically available.

The biomarker panel guiding recommendation for treatment boost and identifying patients who are at risk for relapse early on (after treatment) is very important and could potentially alter the current treatment landscape for cancer, specifically prostate cancer. Monitoring of patients during RT treatment based on their sensitivities to specific RT doses will allow delivery of suitable alternative treatments to high-risk patients and dose escalation to tumors in less sensitive patients during early phases of treatment. The biomarker platform will offer additional benefits of exploring the possibility of use of other modalities (e.g., immunotherapy, molecular targeted therapy) in combination with RT for patients who can't get the maximum benefit from RT alone. FIG. 35 shows the RRI based non-invasive approach allowing longitudinal sample collection from patients prior to, during and after radiation treatment to monitor a continual response to RT and guide clinicians for treatment decisions.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

“One or more,” as used herein, means at least one, and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1-87. (canceled)

88. A method for use in classifying a prostate cancer biological sample or patient as (i) strong responder (SR), (ii) responder (R), (iii) weak responder (WR) or (iv) non-responder (NR) to radiation treatment, wherein the classification provides guidance for dose escalation, de-escalation or alternate therapy or combination therapy before commencing radiation treatment (RT), comprising,
(a) detecting expression level of at least two biomarkers selected from a panel of biomarkers CD63, AR, HIF-1α, P21, Integrin, TP53, AKT, CD81, BRCA1, CD9, RAD51, BCL2, Snail, EGFR, PI3, PD-L1
in the biological sample;
(b) assigning a pre-treatment Radiation Response Index (RRI) index value using a pre-treatment RRI algorithm based on the relative changes or changes in expression of the at least two biomarkers, where a positive RRI value indicates a radiation sensitive tumor (RS-RRI) and a negative RRI value indicates a radiation resistant tumor (RR-RRI); and
(c) classifying a patient based on the RRI value wherein
(i) a high RS-RRI value is a strong responder (SR) and a candidate for reduced RT dose;
(ii) a medium RS-RRI value is a responder (R) and a candidate for the standard RT dose;
(iii) a medium RR-RRI value is a weak responder (WR) and a candidate for dose escalation;
(iv) a low RR-RRI value is a non-responder (NR) and a candidate for dose escalation, or for alternate therapy or combination therapy, in an individualized manner.

89. The method of claim 88, wherein the biological sample is a tissue biopsy, a liquid biopsy, a body fluid, cells, vesicles, exosomes, or a combination thereof.

90. The method of claim 88, wherein the expression level of the at least 2 biomarkers is measured at the nucleic acid and/or protein level in vivo, ex vivo, in vitro, or a combination thereof.

91. The method of claim 88, wherein the biomarker expression being detected is the state of a biomarker molecule including phosphorylation, acylation, alkylation, amidation, glypiation, glycation, glycosylation, ubiquitination, degradation product(s), truncation, mutation status, or binding of the molecule to promoters.

92. The method of claim 88, wherein the biomarker expression being detected is the localization of the biomarker molecule including cellular localization or extracellular localization,
wherein cellular localization comprises intracellular, compartmentalized, nuclear or nucleoli, or membrane bound, and wherein membrane bound includes plasma, nuclear, or other organelle membranes, and compartmentalized localization comprises Golgi, endoplasmic reticulum, lysosomal, endosomal, exosomal, mitochondrial, vacuole, and cytosolic localization.

93. The method of claim 88, wherein the RRI is determined by implementing a system comprising electronic hardware and software that comprises algorithms, and a user interface.

94. A kit for use in the method of claim 88, comprising reagents for determining nucleic acid and/or protein levels of biomarkers CD63, AR, HIF-1α, P21, Integrin, TP53, AKT, CD81, BRCA1, CD9, RAD51, BCL2, Snail, EGFR, PI3, PD-L1.

95. A kit for use in the method of claim 88 comprising reagents for identifying one or more molecular targeted agent (MTA) for use in an alternate therapy or a combination therapy for a patient classified as a weak responder or non-responder, wherein the one or more MTA is a hormone, an agent targeting Snail, an agent targeting one or more of the biomarkers listed in (a), a signal transduction inhibitor, a chemotherapy agent, a biologic therapy, an immunotherapy, a senescence inducer, a polymerase inhibitor, or a combination thereof, wherein the one or more MTA is administered by one or more method including intravenous, intraperitoneal, intra/transdermal, intratumoral, subcutaneous, inhalation, ocular, sublingual, epidural, vaginal, intranodal, transmucosal, and rectal.

96. A method for use in classifying a prostate cancer biological sample or patient as (i) strong responder (SR), (ii) responder (R), (iii) weak responder (WR) or (iv) non-responder (NR) to radiation treatment, wherein the classification provides guidance for dose escalation, de-escalation or alternate therapy or combination therapy during or post radiation treatment (RT) comprising,
(a) detecting in the biological sample expression level of at least one biomarker selected from a panel of biomarkers PI3, P21, CD81, CD63, AR, TP53, HIFα-1, Integrin, RAD51, Snail, CD9, BRCA1, BCL2, EGFR, PD-L1, AKT, wherein PI3 is at least one of the biomarkers;
(b) assigning a post-treatment Radiation Response Index (RRI) value using a post-treatment RRI algorithm based on the relative change or change in expression of the biomarkers listed in (a), where a positive RRI value indicates a radiation sensitive tumor (RS-RRI) and a negative RRI value indicates a radiation resistant tumor (RR-RRI); and
(c) classifying a patient based on the RRI value wherein
(i) a high RS-RRI value is a strong responder (SR) and a candidate for reduced RT dose;
(ii) a medium RS-RRI value is a responder (R) and a candidate for the standard RT dose;
(iii) a medium RR-RRI value is a weak responder (WR) and a candidate for dose escalation;
(iv) a low RR-RRI value is a non-responder (NR) and a candidate for dose escalation, or for alternate therapy or combination therapy, in an individualized manner.

97. The method of claim 96, wherein the biological sample is a tissue biopsy, a liquid biopsy, a body fluid, cells, vesicles, exosomes, or a combination thereof.

98. The method of claim 96, wherein the expression level of the biomarkers is measured at the nucleic acid and/or protein level in vivo, ex vivo, in vitro, or a combination thereof.

99. The method of claim 96, wherein the RRI is determined by implementing a system comprising electronic hardware and software that comprises algorithms, and a user interface.

100. A kit for use in the method of claim 96 to predict radiosensitivity based on RRI, comprising reagents for determining nucleic acid and/or protein levels of biomarkers listed in (a).

101. A kit for use in the method of claim 96 comprising reagents for identifying one or more molecular targeted agent (MTA) for use in an alternate therapy or a combination therapy for a patient classified as a weak responder or non-responder, wherein the one or more MTA is a hormone, a signal transduction inhibitor, an agent targeting one or more of the biomarkers listed in (a), an immunotherapy, a senescence inducer, a polymerase inhibitor, an agent targeting Snail or a combination thereof, wherein the one or more MTA is administered by one or more method including intravenous, intraperitoneal, intra/transdermal, intratumoral, subcutaneous, inhalation, ocular, sublingual, epidural, vaginal, intranodal, transmucosal, and rectal.

102. A method for use in classifying a biological sample or a prostate cancer patient receiving radiation as stable or having a likelihood of relapse post radiation treatment, by assessing development of radiation resistance, comprising
(a) detecting in a biological sample or patient expression level of at least one biomarker selected from a panel of biomarkers PI3, P21, CD81, CD63, AR, TP53, HIFα-1, Integrin, RAD51, Snail, CD9, BRCA1, BCL2, EGFR, PD-L1, AKT, wherein PI3 is at least one of the biomarkers;
(b) assigning a post-treatment Radiation Response Index (RRI) index value using a post-treatment RRI algorithm based on the relative changes or changes in expression of the biomarkers wherein PI3 is at least one of the biomarkers, where a positive RRI value indicates a radiation sensitive tumor (RS-RRI) and a negative RRI value indicates a radiation resistant tumor (RR-RRI); and
(c) classifying the biological sample or patient based on the post-treatment RRI value in (b) as
(i) stable when the tumor is classified as radiation sensitive;
(ii) high likelihood of relapse when the tumor is classified as radiation resistant.

103. The method of claim 102, wherein the biological sample is a tissue biopsy, a liquid biopsy, a body fluid, cells, vesicles, exosomes, or a combination thereof.

104. The method of claim 102, wherein the expression level of the biomarkers is measured at the nucleic acid or protein level in vivo, ex vivo, in vitro, or a combination thereof.

105. The method of claim 102, wherein the biological sample or patient classified as (ii) places the biological sample or patient in a category that can benefit from alternate therapy or combination therapy with one or more molecular targeted agent (MTA) based on the biomarkers, wherein the one or more MTA is a hormone, a signal transduction inhibitor, an agent targeting one or more of the biomarkers listed in (a), an immunotherapy, a senescence inducer, a polymerase inhibitor, an agent targeting Snail, or a combination thereof, wherein the one or more MTA is administered by one or more method including intravenous, intraperitoneal, intra/transdermal, intratumoral, subcutaneous, inhalation, ocular, sublingual, epidural, vaginal, intranodal, transmucosal, and rectal.

106. A kit for use in the method of claim 102 to predict radiosensitivity based on RRI, comprising reagents for determining nucleic acid and/or protein levels of biomarkers listed in (a).

107. A kit for use in method of claim 102 comprising reagents for identifying one or more molecular targeted agent (MTA) for use in one or more alternate therapy or combination therapy for a patient classified as a having high likelihood of relapse wherein the one or more MTA is a hormone, a signal transduction inhibitor, an agent targeting the biomarkers listed in (a), an immunotherapy, a senescence inducer, a polymerase inhibitor, an agent targeting Snail, or a combination thereof, wherein the one or more MTA is administered by one or more method including intravenous, intraperitoneal, intra/transdermal, intratumoral, subcutaneous, inhalation, ocular, sublingual, epidural, vaginal, intranodal, transmucosal, and rectal.
